(19) United States
(12) Patent Application Publication
(10) Pub. No.: US 2021/0002231 A1
(43) Pub. Date: Jan. 7, 2021

(54) HYBRID MU OPIOID RECEPTOR AND NEUROPEPTIDE FF RECEPTOR BINDING MOLECULES, THEIR METHODS OF PREPARATION AND APPLICATIONS IN THERAPEUTIC TREATMENT

(71) Applicants: Centre National de la Recherche Scientifique, Paris (FR); Université de Strasbourg, Strasbourg (FR); Vrije Universiteit Brussel, Brussel (BE)

(72) Inventors: Frédéric Simonin, ILLKIRCH GRAFFENSTADEN (FR); Armand Drieu la Rochelle, SAINT-GRÉGOIRE (FR); Frédéric Bihel, FEGERSHEIM (FR); Steven Ballet, ITEGEM (BE)

(21) Appl. No.: 16/979,353

(22) PCT Filed: Mar. 11, 2019
(86) PCT No.: PCT/EP2019/056054

§ 371 Date: Sep. 9, 2020

(30) Foreign Application Priority Data
Mar. 9, 2018 (EP) 18305256.2

(51) Int. Cl. C07D 223/14; A61K 38/04

(52) U.S. Cl. CPC C07D 223/14; A61K 38/04

(57) ABSTRACT

Molecules binding the mu opioid receptor (MOR) and the neuropeptide FF receptor (NPFFR), in particular molecules having a MOR agonist and NPFFR modulatory activity, and pharmaceutical compositions useful in the treatment of pain and/or hyperalgesia.

The present invention concerns molecules binding to mu opioid receptor (MOR) and neuropeptide FF receptor (NPFFR) and compositions containing said molecules and their applications in therapeutic treatment.

### STATE OF THE ART

Opioids analgesics, such as morphine and fentanyl, continue to be the cornerstone drugs for treating moderate to severe pain. Although undeniable benefit for treating acute severe pain, opioids still lack the demonstration of effectiveness for long-term therapy. Indeed, repeated administration of morphine exacerbate its adverse effects such as pain hypersensitivity, which in turn impair the analgesic efficacy (tolerance) and trigger a dose-escalating downward spiral. In addition to pain hypersensitivity, risks of respiratory depression, addiction and severe constipation are contributing to degrade patient quality of life. In the pursuit of safer analgesics, two main strategies recently emerged: G-protein biased MOP (μ opioid) receptor agonists and multi-functional drugs with mixed opioid and non-opioid activities (Gunther et al., 2017: Gunther, T., Dasgupta, P., Mann, A., Miess, E., Kliewer, A., Fritzwanker, S., Steinborn, R., Schulz, S., 2017. Targeting multiple opioid receptors—improved analgesics with reduced side effects?, Br. J. Pharmacol. 2017 Apr. 5. doi: 10.1111/bph.13809; Olson et al., 2017: Olson, K. M., Lei, W., Keresztes, A., LaVigne, J., Streicher, J. M., 2017. Novel Molecular Strategies and Targets for Opioid Drug Discovery for the Treatment of Chronic Pain. Yale J Biol Med 90, 97-110).

Opioid research for understanding the cellular mechanisms of the physiological analgesic response unveiled multiple pathways for MOP receptor signaling. The coupling to heterotrimeric Gai/o inhibits cAMP production by adenylyl cyclases and is ultimately responsible for pain relief. On the other side, β-arrestin binding to MOP receptor induces internalization, desensitization as well as β-arrestin specific signaling, which has been widely reported to impair the pain relief process and to be mainly responsible for the opioid-induced respiratory depression and constipation. The benefit of G-protein biased MOP receptor agonists, such as TRV130, has been extensively studied with promising results in acute administration. However, the consequences of chronic administration of such compounds have been poorly studied.

Another strategy has recently emerged to develop new analgesic drugs with an improved therapeutic profile. Instead of focusing on highly selective biased MOP agonists, dual acting drugs both bind the MOP receptors to produce analgesia as well as other biological targets relevant at alleviating opioid side effects. In addition to MOP receptor, such compounds bind other opioid receptors or non-opioid receptors (Gunther et al., 2017; Olson et al., 2017): MOP/DOP dual agonist MGM-16, MOP/NOP dual agonist BU08028 or dual MOP-NK1 antagonist have shown improved acute analgesic profiles with reduced side effects in various acute and chronic pain models. Among the non-opioid receptors, neuropeptide FF (NPFF) receptors have been identified to participate to the development of opioid-induced analgesic tolerance and other adverse effects (Ayachi and Simonin, 2014: Ayachi, S., Simonin, F., 2014. Involvement of Mammalian RF-Amide Peptides and Their Receptors in the Modulation of Nociception in Rodents. Front Endocrinol (Lausanne) 5, 158; Simonin, 2006: Simonin, F., 2006. Neuropeptide FF receptors as therapeutic targets. Drugs Future 31, 603-609).

NPFF 1 and 2 receptor subtypes belong to the family of RF-amide peptide receptors and are mainly coupled to the G-protein Gi/o (Quillet et al., 2016: Quillet, R., Ayachi, S., Bihel, F., Elhabazi, K., (lien, B., Simonin, F., 2016. RF-amide neuropeptides and their receptors in Mammals: Pharmacological properties, drug development and main physiological functions. Pharmacol Ther 160, 84-132). The involvement of the NPFF system in the modulation of nociception and opioid analgesia has been largely studied. Because NPFF produces transient hyperalgesia, attenuates the analgesic effect of morphine and potentiates the overall morphine withdrawal syndrome, it has been described as an anti-opioid peptide (Ayachi and Simonin, 2014). Moreover, pharmacological blockade of NPFF1/2 receptors has been shown to prevent the development of opioid-induced hyperalgesia (01H), and analgesic tolerance as well as to reduce the morphine withdrawal syndrome.

In light of these findings, there is a clear need to identify new molecules having analgesic activity but limiting one or more of the clinical complications of opioid-induced hyperalgesia, analgesic tolerance and opioid withdrawal syndrome. A solution to these problems is even more important to tackle the present opioid epidemic worldwide (Grosser et al., 2017: Grosser, T., Woolf, C. J., FitzGerald, G. A., 2017. Time for nonaddictive relief of pain. Science 355, 1026-1027).

### Aims of the Invention

The present invention aims to solve the technical problem consisting of providing molecules having a MOR agonist activity.

The present invention aims to solve the technical problem consisting of providing molecules having a NPFF antagonist, partial agonist or agonist activity.

The present invention aims to solve the technical problem consisting of providing molecules having combined MOR agonist activity and a NPFF antagonist activity.

The present invention aims to solve the technical problem consisting of providing molecules having combined MOR agonist activity and a NPFF partial agonist activity.

The present invention aims to solve the technical problem consisting of providing molecules having combined MOR agonist activity and a NPFF agonist activity.

The present invention aims to solve the technical problem consisting of providing molecules having an analgesic activity, in particular upon acute administration.

The present invention aims to solve the technical problem consisting of providing molecules limiting or preventing opioid-induced hyperalgesia (01H).

The present invention aims to solve the technical problem consisting of providing molecules limiting or preventing analgesic tolerance.

The present invention aims to solve the technical problem consisting of providing molecules reducing the morphine withdrawal syndrome.

### DETAILED DESCRIPTION OF THE INVENTION

The present inventors have discovered a new class of molecules providing a solution to one or more of the technical problem recited in the present invention.

The invention relates in particular to molecules comprising the following structure:

X1-X2-X3-X4-X5-X6-T

wherein
X1 has the following structure:

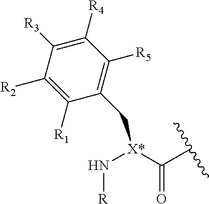

wherein
R₂, R₃ and R₄ are, independently at each occurrence H, OH (para position preferred), NH₂, CH₃, CONH₂, or COCH₃
R₁ and R₅ are, independently at each occurrence, H or Me
R is H, alkyl or C(═N)NH₂
X is N or CRa, wherein Ra is H or Me;
X2 is a natural or non-natural amino acid residue, or derivative thereof, including homologated amino acids, aza amino acids,
or X1-X2 represent together the following structure:

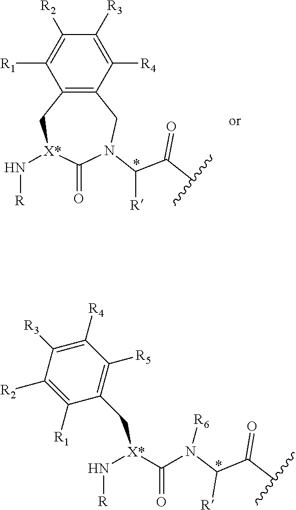

R₂, R₃ and R₄ are, independently at each occurrence H, OH, NH₂, CH₃, CONH₂, or COCH₃

R₁, R₅ and R₆ are, independently at each occurrence, H or alkyl (preferably Me)

R is H or C(═N)NH₂

R′ is H or a group of atoms, including natural and non-natural amino acid side chains

X is N or CRa, wherein Ra is H or Me,
X3 is a natural or non-natural amino acid residue,
X4 is one to five natural or non-natural amino acid residues or a derivative thereof,
X5 has the following structure:

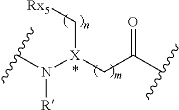

wherein Rx₅ is a cyclic or acyclic tertiary amine group linked by the nitrogen atom of the tertiary amine group,

wherein: if m=0, then n=2, 3, or 4;
if m=1, or 2, then n=1

wherein X is CRa or N, wherein Ra is H or Me,

wherein R′ is H or Alkyl (for example Me, Et);

wherein each carbon atom of (CH₂)n and (CH₂)m can be substituted independently of each other;
X6 is a natural or non-natural amino acid residue
T is a chemical terminal group of atoms,

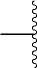

represents a covalent link.

According to the present invention, * possibly represents R or S configuration for a chiral atom, unless stated otherwise.

In one embodiment, in X1, R₂ and R₄ are H and R₃ is OH.

In one embodiment, X1 is:

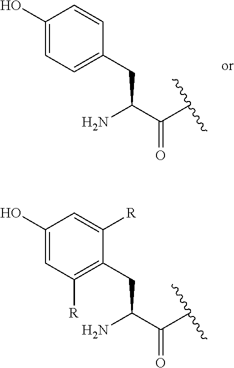

wherein R is alkyl (for example methyl, ethyl),

or

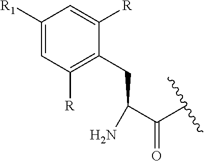

wherein R₁ is NH₂, CH₃, CONH₂, COCH₃ and R is H or alkyl (methyl, ethyl).

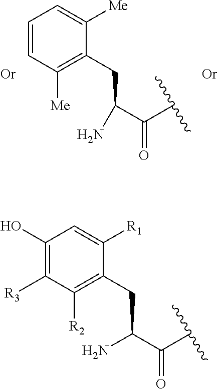

wherein:

R₁ is Me (methyl), R₂ is Et (ethyl) and R₃ is H or D

R₁, R₂ and R₃ are Me.

In one embodiment, X2 is:

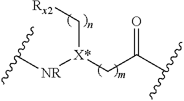

wherein X is CRa or N and Ra is H or Me;

wherein R is H or alkyl (typically Me);

wherein Rₓ₂ is H, alkyl chain bearing an substituted amino group or a substituted guanidine group,

wherein each carbon atom of (CH₂)n and (CH₂)m can be substituted independently of each other,

wherein: if m=0, then n=2, 3, or 4;
if m=1, or 2, then n=1.

In one embodiment, X2 is:

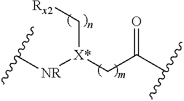

wherein X is CRa or N and Ra is H or Me;

wherein R is H or alkyl (typically Me);

wherein Rₓ₂ is a cyclic or acyclic tertiary amine group linked by the nitrogen atom of the tertiary amine group or is a phenyl group optionally substituted by an alkyl group or an amino acid side chain,

wherein each carbon atom of (CH₂)n and (CH₂)m can be substituted independently of each other,

wherein: if m=0, then n=2, 3, or 4;
if m=1, or 2, then n=1.

wherein each carbon atom of (CH₂)n and (CH₂)m can be substituted independently of each other.

In one embodiment of X2, X is CH or N and when X is CH, C can be asymmetric (X*).

In one embodiment, X2 has the following structure:

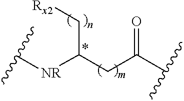

wherein Rₓ₂ is a cyclic or acyclic tertiary amine group linked by the nitrogen atom of the tertiary amine group or is a phenyl group optionally substituted by an alkyl group or an amino acid side chain,

wherein R is H or alkyl (typically Me);

wherein: if m=0, then n=2, 3, or 4;
if m=1, or 2, then n=1.

wherein each carbon atom of (CH₂)n and (CH₂)m can be substituted independently of each other.

In one embodiment X2 is:

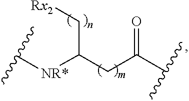

and for example X2 is selected from the group consisting of:

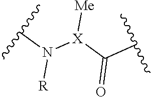

wherein R is H or alkyl (typically Me) and X is CH or N;

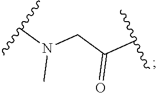

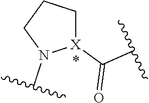

wherein X is CH or N;

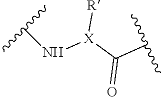

wherein R′ is an amino acid side chain and X is CH or N;

In one embodiment X2 is Arg (arginine) and derivatives thereof (surrogates) such as for example:

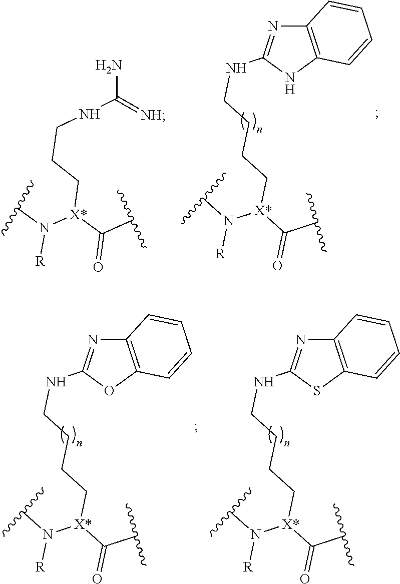

wherein R is H or alkyl (typically Me), wherein n=0 or 1, wherein each carbon atom of (CH₂)n and (CH₂)m can be substituted independently of each other.

In one embodiment X2 is Lys (lysine) and derivatives thereof (surrogates) such as for example:

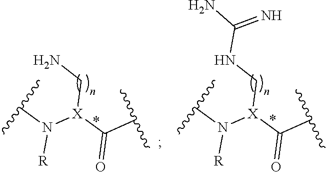

wherein n is 0 to 5,

wherein R is H or alkyl (typically Me) wherein each carbon atom of (CH2)n and (CH2)m can be substituted independently of each other.

An example of a cyclic or acyclic ring for X1X2 is:

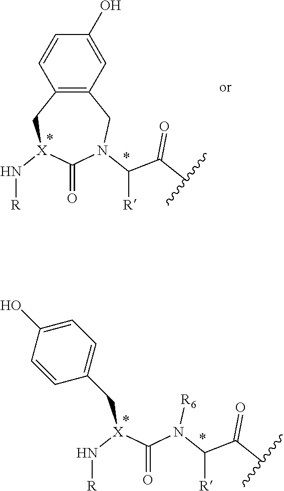

wherein R₆ is H or Me

wherein R is H or C(═N)NH₂

wherein R′ is a H or a group of atoms, including natural and non-natural amino acid side chains.

In one embodiment of X1X2, X is CH.

In one embodiment, X1X2 represents:

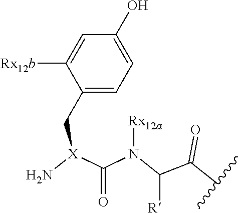

wherein Rx₁₂ₐ and Rx_{12b} are, independently at each occurrence, H, Me or form together a cyclic ring,

wherein R′ is a H or a group of atoms, including natural and non-natural amino acid side chains.

In one embodiment, X3 has the following structure:

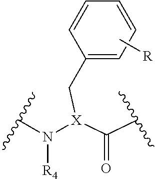

wherein R4 is an amino acid side chain; and wherein R is one or more substituents and preferably each independently selected from H, halogen, alkyl, alkenyl and (hetero)aryl;

wherein X is CRa or N, wherein Ra is H or Me.

In one embodiment, X3 has the following structure:

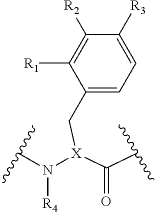

wherein R4 is an amino acid side chain; R₁, R₂ and R₃ are each independently H, halogen, alkyl, alkenyl, (hetero)aryl, aromatic can be mono-/di-/trisubstituted;

wherein X is CRa or N, wherein Ra is H or Me.

In one embodiment, X3 is:

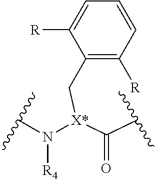

wherein R is at each occurrence independently H or an alkyl (typically Me or Et), wherein X is CRa or N, wherein Ra is H or Me and wherein R4 is an amino acid side chain.

In one embodiment, X3-X4 together represent the following structure:

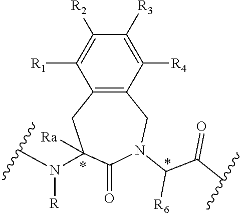

Wherein R is an amino acid side chain
Ra is H or Me
R₁ to R₄ and R₆ are each independently H, halogen, alkyl, alkenyl, (hetero)aryl;

or

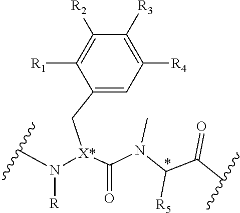

wherein X is CRa or N, wherein Ra is H or Me

wherein R₄ is an amino acid side chain
Ra is H or Me
R₁ to R₅ are each independently H, halogen, alkyl, alkenyl, (hetero)aryl.

In one embodiment, X3-X4 together represent

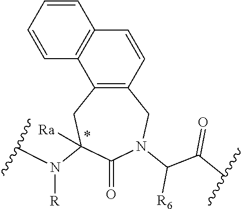

wherein R is an amino acid side chain; Ra is H or Me; R₆ is H, halogen, alkyl, alkenyl or (hetero)aryl.

In one embodiment, X3X4 represents:

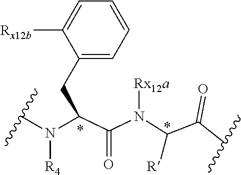

wherein Rx₁₂ₐ and Rx_{12b} are, independently at each occurrence, H, Me or form together a cyclic ring,

wherein R is H or Alkyl (for example Me, Et) and R′ is a H or a group of atoms, including natural and non-natural amino acid side chains.

In one embodiment, X3X4 represents:

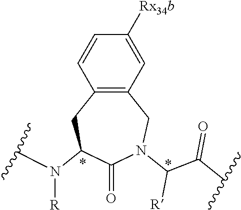

wherein Rx_{34b} is H or Alkyl (for example Me, Et); wherein R is H or Alkyl (for example Me, Et) and R′ is a H or a group of atoms, including natural and non-natural amino acid side chains.

In one embodiment, X4 has the following structure:
α-amino acids

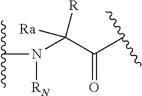

wherein R is an amino acid side chain or derivative thereof

wherein Ra is H or Me

wherein R_{N} is H or alkyl
β3-homo-amino acids

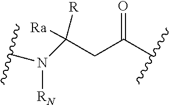

wherein R is an amino acid side chain or derivative thereof

wherein Ra is H or Me

wherein R_{N} is H or alkyl
α2-homo-amino acids

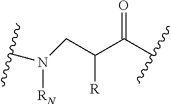

wherein R is an amino acid side chain or derivative thereof

wherein R_{N} is H or alkyl
aza-amino acids

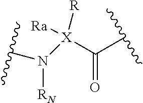

wherein R is an amino acid side chain or derivative thereof

wherein Ra is H or Me

wherein R_{N} is H or alkyl.

In one embodiment, X4 is one to five natural or non-natural amino acid residue optionally including a modified C-terminus. In case of a modified C-terminus, X4 is a derivative of an amino acid.

In one specific embodiment, X4 comprises one of the following C-terminal group forming a natural or non-natural amino acid residue or a derivative thereof:

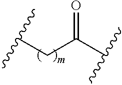

wherein 0≤m≤5;

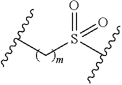

wherein 0≤m≤5;

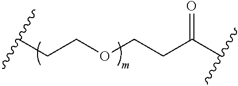

wherein 1≤m≤3;

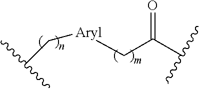

wherein 0≤m≤3 and 0≤n≤3;

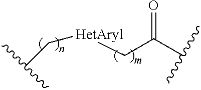

wherein 0≤m≤3 and 0≤n≤3;

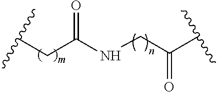

wherein 0≤m≤5 and 0≤n≤5

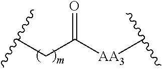

wherein 0≤m≤5 and AA₃ represents one or two residues selected each independently among the list of residues X5 and X6, wherein each carbon atom of (CH₂)n and (CH₂)m can be substituted independently of each other.

In one embodiment, X4 is a natural or non-natural amino acid derivative as it could be linked through a SO₂ function at the N-terminus of X5.

In one embodiment, X5 is:

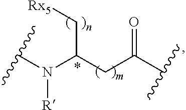

wherein each carbon atom of (CH₂)n and (CH₂)m can be substituted independently of each other.

In one embodiment, X5 is:

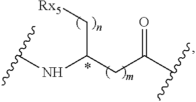

wherein each carbon atom of (CH₂)n and (CH₂)m can be substituted independently of each other.

In one specific embodiment, Rₓ₅ is selected from the group consisting of:
a cyclic or acyclic guanidine bearing various substituents such as:

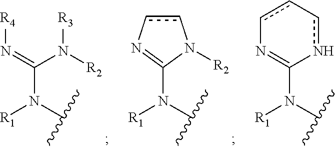

a cyclic or acyclic urea or thiourea bearing various substituents such as:

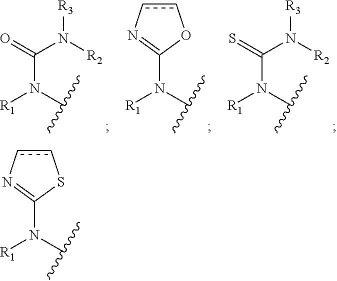

a cyclic or acyclic tertiary amine group such as:

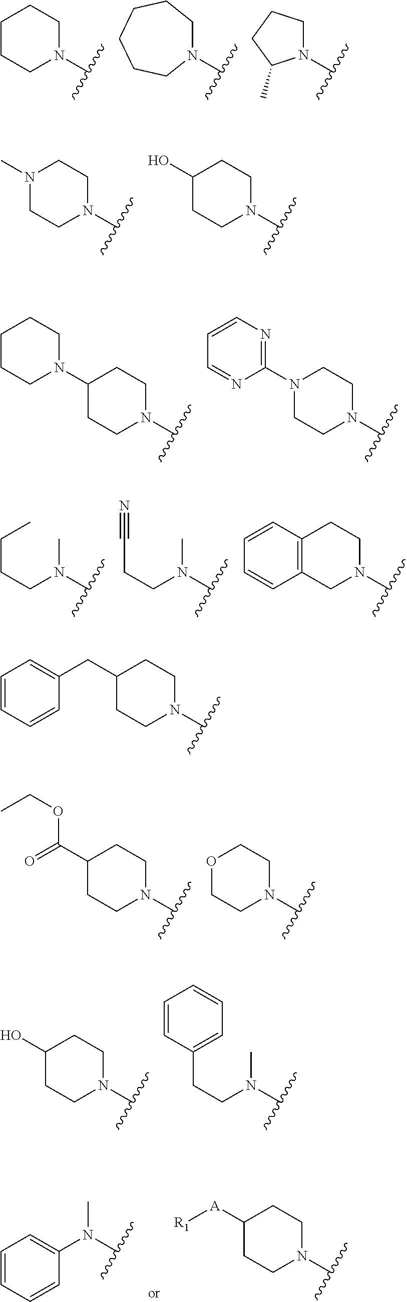

wherein A is (CH₂)ₙ with n=0, 1, 2, 3, O, S, or NH

wherein each carbon atom of (CH₂)n and (CH₂)m can be substituted independently of each other,

wherein R1 is Aryl or Heteroaryl, said Aryl or Heteroaryl possibly bearing various substitutions including:
>Alkoxy, Alkyl, Amines, etc
Cyclic or acyclic Alkyl chains. In one specific embodiment, Rₓ₅ is a cyclic or acyclic guanidine bearing various substituents such as:

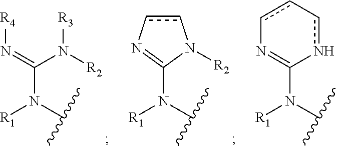

In one specific embodiment, Rₓ₅ is:

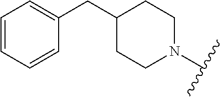 [SMILES: *N1CCC(Cc2ccccc2)CC1]

In one embodiment, X6 is selected from the group consisting of:
a natural or non-natural amino acids of configuration L or D including one of the following structures:
   Gly, Ala, Val, Ile, Leu, Nle, cHex, Phe, Hphe, Tyr, Trp, Asn, Gln, Pro
   Arg, Lys, Cys, Met, Asp, Glu
   a bridged amino acids including:

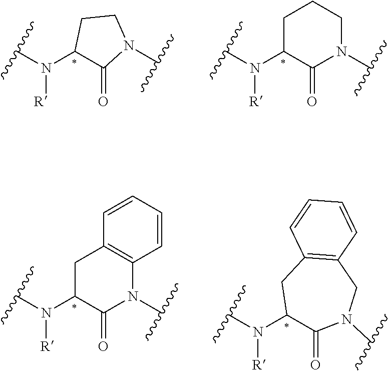

wherein R′ is H or Alkyl (for example Me, Et).

In one embodiment, X6 is selected from the group consisting of the following structures:

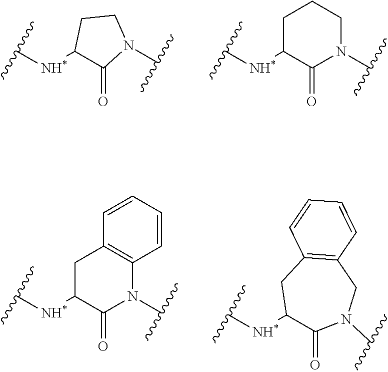

In one embodiment, said terminal group T is selected from the group consisting of:
H, Alkyl, (CH₂)ₙ-Aryl (when X6 is a bridged amino acid), or
NH₂, NH—R or cyclic/acyclic NR₁R₂ wherein R, R₁, R₂ is H, Alkyl or (CH₂)ₙ-Aryl.

In one specific embodiment, said terminal group T is NH₂.

In one embodiment, said terminal group T is a fluoroalkyl, and for example (CH₂)ₙ—(CF₂)ₘ—CF₃, wherein n and m are integers, typically ranging independently from 0 to 10.

In one embodiment, said terminal group T is a polyethyleneglyocl (PEG).

Typically, said molecule comprises from 6 to 10 amino acid residues or derivative thereof.

In one embodiment, said compound represents the following structure:

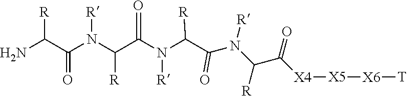

wherein R is at each occurrence independently H or a group of atoms, including natural and non-natural amino acid side chains;

wherein R′ is at each occurrence independently H or alkyl (Me or Et preferred).

In one embodiment, according to the invention, a natural or non-natural amino acids of configuration L or D includes:

Gly, Ala, Val, Ile, Leu, Nle, cHex, Phe, Hphe, Tyr, Trp, Asn, Gln, Pro, and non-natural derivatives thereof.

In one embodiment, according to the invention, a natural or non-natural amino acids of configuration L or D includes:

Arg, Lys, Cys, Met, Asp, Glu, and non-natural derivatives thereof.

In one embodiment, X1 is H-Dmt (2,6-Dimethyltyrosine).

In one embodiment, X2 is D-Arg (Arginine).

In one embodiment, X2 is Arg, Pro, Bpa (Rₓ₅ is 4-Benzyl-phenylalanine), N(Me)Ala, Orn, Lys, hArg, Lys(Nic), or NLys.

Preferably, X2 is a natural or non-natural amino acid having D configuration.

In one embodiment, X2 is D-Arg, D-hArg, D-Orn, D-Lys, D-Lys(Tic), N(Me)-D-Ala, or D-Pro.

In one embodiment, X3 is Aba (4-Amino-tetrahydrobenzazepinone and more specifically (4S)-4-amino-1,2,4,5-tetrahydro-2-benzazepin-3-one).

In one embodiment, X3 is Aba, Phe, or Ana ((2S)-2-amino-1H,2H,4H,5H-naphtho[2,1-c]azepin-3-one).

In one embodiment, X4 is Gly or -β-Ala (glycine or beta-alanine).

In one embodiment, X4 is Gly, N(Me)Gly, Ala, N(Me)Ala, GABA (gamma-aminobutyrique acid).

In one embodiment, X5 is Arg, Orn, Bpa, Lys or a Lys derivative such as for example Lys(Bim), Lys(Box) or Lys(Bth) (Arginine, Ornithine, 4-Benzoylphenylalanine, Phenylalanine; Lysine).

In one embodiment, X5 is Tetrahydroisoquinoline (THIQ).

In one embodiment, X5 is Bpa ((2S)-2-amino-5-(4-benzylpiperidin-1-yl)pentanoic acid).

In one embodiment, X5 is Arg.

In one embodiment, X6 is Phe, Val, Ile, Leu Tyr or Trp.

In one embodiment, X6 is Phe (Phenylalanine).

In one embodiment, X6 is D-Phe In one embodiment, X6-T is Phe-NH₂.

Preferably, X6 is a natural or non-natural amino acid having D configuration.

In one embodiment, the molecule of the present invention comprises the sequence:
Arg-Phe-NH₂ (X5-X6-T).

In one embodiment, the molecule of the present invention comprises the sequence:
Bpa-Phe-NH₂ (X5-X6-T).

In one embodiment, the molecule of the present invention comprises a sequence (X5-X6-T) selected among: Bpa-Val-NH₂, Bpa-Ile-NH₂, Bpa-Leu-NH₂, Bpa-Tyr-NH₂, and Bpa-Trp-NH₂.

In one embodiment, the molecule of the present invention comprises a sequence (X1-X2-X3) H-Dmt-Arg-Aba.

In one embodiment, the molecule of the present invention comprises a sequence (X1-X2-X3-X4) H-Dmt-Arg-Aba-Ala.

In one embodiment, said compound represents the following structure:

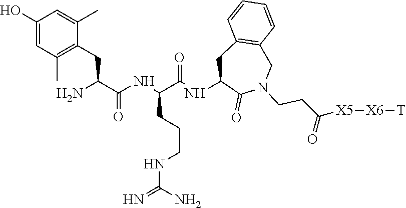

At any occurrence of alkyl, alkenyl or (hetero)aryl, such groups can be mono-/di-/trisubstituted.

Unless otherwise specified, the below terms used herein are defined as follows. As used herein, the terms “alkyl” and “alkenyl” mean in particular a saturated straight chain or branched non-cyclic hydrocarbon having from 1 to 10 carbon atoms, preferably from 1 to 6 carbon atoms, more preferably from 1 to 4 carbon atoms. Representative saturated straight chain alkyls include methyl, ethyl, n-propyl, n-butyl; while saturated branched alkyls include isopropyl, sec-butyl, isobutyl, tert-butyl, and the like. Alkyl and alkenyl groups included in compounds of this invention may be optionally substituted with one or more substituents.

Derivatives of alkyls and alkenyls comprise alkoxy, alkenoxy, thioalkyls and thioalkenyls.

Substituted alkyls and alkenyls comprise haloalkyls and haloalkenyls.

As used herein, the term “alkoxy” and “alkenoxy” refers to an alkyl group as defined above which is attached to another moiety by an oxygen atom. Examples of alkoxy groups include methoxy, isopropoxy, ethoxy, tert-butoxy, and the like. Alkoxy groups may be optionally substituted with one or more substituents. Alkoxy groups included in compounds of this invention may be optionally substituted with a solubilising group.

At any occurrence of alkyl, preferred alkyl groups are methyl (Me) and ethyl (Et). In one embodiment, alkyl or alkenyl is methyl.

As used herein, the term “thioalkyl” or “thioalkenyl” refers to an alkyl or alkenyl group as defined above which is attached to another moiety by a sulfur atom. Thioalkyl groups may be optionally substituted with one or more substituents. Thioalkyl groups included in compounds of this invention may be optionally substituted with a solubilising group. As used herein, the term “heterocycle” refers collectively to heterocycloalkyl groups and heteroaryl groups.

As used herein, the term “haloalkyl” or “haloalkenyl” means an alkyl or alkenyl group as defined above in which one or more (including all) the hydrogen radicals are replaced by a halo group, wherein each halo group is independently selected from —F, —Cl, —Br, and —I. The term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like. Haloalkyl and or haloalkenyl groups may be optionally substituted with one or more substituents.

As used herein, the term “haloalkoxy” or “haloalkenoxy” means an alkoxy or alkenoxy group as defined above in which one or more (including all) the hydrogen radicals are replaced by a halo group, wherein each halo group is independently selected from —F, —Cl, —Br, and —I. Representative haloalkoxy groups include trifluoromethoxy, bromomethoxy, 1,2-dichloroethoxy, 4-iodobutoxy, 2-fluoropentoxy, and the like. Haloalkoxy groups may be optionally substituted with one or more substituents.

As used herein, the term “aryl” means a monocyclic or polycyclic-aromatic radical comprising carbon and hydrogen atoms. Examples of suitable aryl groups include, but are not limited to, phenyl. An aryl group can be unsubstituted or substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. A heteroatom may be substituted with a protecting group known to those of ordinary skill in the art, for example, the hydrogen on nitrogen may be substituted. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

As used herein the term “substituent” or “substituted” means that a hydrogen radical on a compound or group is replaced with any desired group that is substantially stable to reaction conditions in an unprotected form or when protected using a protecting group.

In one embodiment, each carbon atom of (CH₂)n and/or (CH₂)m is not substituted and thus represents CH₂.

In one embodiment, each carbon atom of (CH₂)n and/or (CH₂)m is independently CH₂ or substituted by one or two methyl group.

Examples of compounds according to the invention are the following:

(KGFF01)
H-Dmt-D-Arg-Aba-Gly-Arg-Phe-NH₂;
(KGFF02)
H-Dmt-D-Arg-Aba-Gly-Arg-Phe-OH;
(KGFF03)
H-Dmt-D-Arg-Aba-β-Ala-Arg-Phe-NH₂;
(KGFF04)
H-Dmt-D-Arg-Aba-Gly-Orn-Phe-NH₂;
(KGFF08)
H-Dmt-D-Arg-Aba-β-Ala-Apa-Phe-NH₂;
(KGFF09)
H-Dmt-D-Arg-Aba-β-Ala-Bpa-Phe-NH₂;
(KGFF14)
H-Dmt-D-Arg-Aba-β-Ala-Lys(Bim)-Phe-NH₂;
(KGFF15)
H-Dmt-D-Arg-Aba-β-Ala-Lys(Box)-Phe-NH₂;
(KGFF16)
H-Dmt-D-Arg-Aba-β-Ala-Lys(Bth)-Phe-NH₂;

or a derivative thereof.

Examples of compounds according to the invention are the following:

(DP0001)
H-Dmt-D-Arg-Aba-β-Ala-Bpa-Val-NH₂;
(DP0002)
H-Dmt-D-Arg-Aba-β-Ala-Bpa-Ile-NH₂;
(DP0003)
H-Dmt-D-Arg-Aba-β-Ala-Bpa-Leu-NH₂;
(DP0004)
H-Dmt-D-Arg-Aba-β-Ala-Bpa-Tyr-NH₂;
(DP0005)
H-Dmt-D-Arg-Aba-β-Ala-Bpa-Trp-NH₂;
(DP0007)
H-Dmt-N(Me)-D-Ala-Aba-β-Ala-Bpa-Phe-NH₂;
(DP0008)
H-Dmt-D-Pro-Aba-β-Ala-Bpa-Phe-NH₂;
(DP0009)
H-Dmt-D-Bpa-Aba-β-Ala-Bpa-Phe-NH₂;
(DP0012)
H-Dmt-D-Arg-1AnaGly-Bpa-Phe-NH₂;
(DP0013)
H-Dmt-D-Arg-Phe-N(Me)-β-Ala-Bpa-Phe-NH₂;
(DP0014)
H-Dmt-N(Me)-D-Ala-1AnaGly-Bpa-Phe-NH₂;
(DP0015)
H-Dmt-D-Arg-Aba-β-Ala-Bpa-D-Phe-NH₂;
(DP0016)
H-Dmt-D-Arg-Aba-β-Ala-Bpa-Phe-β-Ala-NH₂;
(DP0017)
H-Dmt-N(Me)-D-Ala-AbaGABA-Bpa-Phe-NH₂;
(DP0018)
H-Dmt-D-Arg-AbaGABA-Bpa-Phe-NH₂;
(DP0019)
H-Dmt-D-Arg-Phe-β-Ala-Bpa-Phe-NH₂;
(DP0020)
H-Dmt-D-Arg-AbaGABA-Bpa-Val-NH₂;
(DP0021)
H-Dmt-D-Arg-1AnaGly-Bpa-Val-NH₂;
(DP0022)
H-Dmt-D-Arg-AbaGABA-Bpa-Trp-NH₂;
(DP0023)
H-Dmt-D-Arg-1AnaGly-Bpa-Trp-NH₂;
(DP0024)
H-Dmt-D-Arg-Phe-N(Me)Gly-Bpa-Phe-NH₂;
(DP0025)
H-Dmt-D-Arg-Phe-N(Me)Gly-Bpa-Val-NH₂;
(DP0026)
H-Dmt-D-Arg-Aba-β-Ala-THIQ-Phe-NH₂;
(DP0027)
H-Dmt-D-Arg-Aba-β-Ala-D-Bpa-Phe-NH₂;
(DP0028)
H-Dmt-D-Orn-Aba-β-Ala-Bpa-Phe-NH₂;
(DP0029)
H-Dmt-D-Lys-Aba-β-Ala-Bpa-Phe-NH₂;
(DP0030)
H-Dmt-D-Arg-Phe-N(Me)-D-Ala-Bpa-Phe-NH₂;
(DP0031)
7-OH-Tic-D-Arg-Aba-β-Ala-Bpa-Phe-NH₂;
(DP0032)
Guanidyl-Dmt-D-Arg-Aba-β-Ala-Bpa-Phe-NH₂;
(DP0033)
H-Dmt-D-hArg-Aba-β-Ala-Bpa-Phe-NH₂;
(DP0034)
H-Dmt-D-Lys(Nic)-Aba-β-Ala-Bpa-Phe-NH₂;
(DP0035)
H-Dmt-Nlys-Aba-β-Ala-Bpa-Phe-NH₂;

or a derivative thereof.

In one embodiment, said compound represents the following structure (KGFF09):

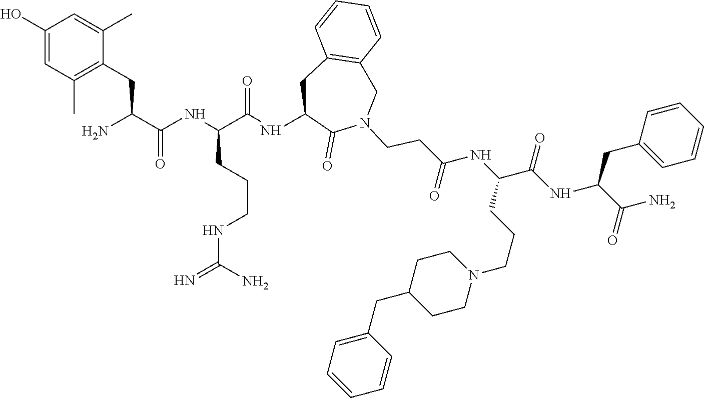 [SMILES: Cc1cc(O)cc(C)c1C[C@H](N)C(=O)N[C@H](CCCNC(=N)N)C(=O)N[C@H]1Cc2ccccc2CN(CCC(=O)N[C@@H](CCCN2CCC(Cc3ccccc3)CC2)C(=O)N[C@@H](Cc2ccccc2)C(N)=O)C1=O]

and derivatives thereof.

The invention also relates to a method for preparing one or more molecules according to the invention.

In one embodiment, the molecules of the invention are prepared by conventional peptide synthesis. In one embodiment, the molecules of the invention are prepared by preparing building blocks X1, X2, X3, X4, X5 and/or X6 prior to conventional solid-phase peptides synthesis (SPPS). For example, the building blocks are prepared in solution before assembly of the peptide sequence (including peptide analogues).

In one embodiment, said method comprises the peptide synthesis of said molecule starting by C-terminal X6.

In one embodiment, the molecules of the present invention present the combination of an opioid residue and an NPFF residue. More particularly, X1-X2-X3-X4 represent a opioid peptide-based peptide analogue structure, for example a dermorphin peptide based peptide analogue structure.

Advantageously, molecule according to the present invention, wherein said molecule is binding MOR and NPFFR.

Preferably, said molecule is an opioid agonist, and in particular a MOR agonist.

Preferably, said molecule is an NPFFR1 or NPFFR2 antagonist, and in particular a NPFFR1 and NPFFR2 antagonist.

In particular, the resent invention relates to analgesic molecules, preferably with reduced side effects in particular after repeated administration.

In one embodiment, the invention relates to molecules activating G protein. In one embodiment, the invention relates to molecules preferentially activating G protein over the β-arrestin2 recruitment. In one embodiment, the invention relates to molecules as G-protein biased MOP receptor agonists.

In one embodiment, the molecules of the present invention have an affinity to NOP receptor.

In one embodiment, the molecules of the present invention have an affinity to KOP receptor.

In one embodiment, the molecules of the present invention have an affinity to DOP receptor.

In one embodiment, the molecules of the present invention are KOP receptor antagonists.

In one embodiment, the molecules of the present invention are DOR receptor agonists.

The invention also relates to, a molecule according to the present invention for use in a method for treating an animal or human body by administration to said body of an effective amount of said molecule.

In one embodiment, said molecule is for use in a method of treatment of pain and/or hyperalgesia.

In one embodiment, said molecule is for use in a method of treatment of a disease or condition associated with MOR.

In one embodiment, said molecule is for use in a method of treatment of a disease or condition associated with NPFFR1 and/or NPFFR2.

In one embodiment, said molecule is for treating pain, for example persistent inflammatory pain.

In one embodiment, said molecule is for use in a method of treatment of behavioral and somatic signs of opioid withdrawal syndrome.

The present invention also relates to a pharmaceutical comprising at least one molecule according to any one of claims 1 to 19 and one or more pharmaceutically acceptable excipients.

The invention relates to the use of a molecule according to the invention for preparing a pharmaceutical composition.

In one embodiment, said pharmaceutical composition is for the administration to an animal or human body of an effective amount of said molecule.

In one embodiment, said pharmaceutical composition is for treating pain, for example persistent inflammatory pain.

In one embodiment, said pharmaceutical composition is for treating of pain.

In one embodiment, said pharmaceutical composition is for treating of hyperalgesia.

In one embodiment, said pharmaceutical composition is for treating of a disease or condition associated with MOR.

In one embodiment, said pharmaceutical composition is for treating of a disease or condition associated with NPFFR1 and/or NPFFR2.

In one embodiment, said pharmaceutical composition is for treating behavioral and somatic signs of opioid withdrawal syndrome.

The invention relates to a method of therapeutic treatment, said method comprising administering to an animal or a human subject an effective amount of said molecule, preferably formulated as a pharmaceutical composition, to a subject in need thereof.

In one embodiment, said method is for treating of pain.

In one embodiment, said method is for treating of hyperalgesia.

In one embodiment, said method is for treating of a disease or condition associated with MOR.

In one embodiment, said method is for treating of a disease or condition associated with NPFFR1 and/or NPFFR2.

The invention relates to a method of therapeutic treatment or relates to said pharmaceutical composition or to the use of a molecule according to the invention for preparing a pharmaceutical composition for use in a method of therapeutic treatment, wherein said therapeutic treatment is for treating pain, a gastrointestinal disorder, or an inflammatory bowel disorder, wherein said method comprises administering to an animal or a human subject an effective amount of a molecule according to the invention, preferably formulated as a pharmaceutical composition, to a subject in need thereof.

The invention relates to a method of therapeutic treatment or relates to said pharmaceutical composition or to the use of a molecule according to the invention for preparing a pharmaceutical composition for use in a method of therapeutic treatment, wherein said therapeutic treatment is for treating a cardiovascular disorder or a neuroendocrine disease, wherein said method comprises administering to an animal or a human subject an effective amount of a molecule according to the invention, preferably formulated as a pharmaceutical composition, to a subject in need thereof.

In one embodiment, said method is for treating behavioral and somatic signs of opioid withdrawal syndrome.

In one embodiment, said method is for treating or limiting respiratory depression, especially in a method of treatment of pain.

In one embodiment, said pharmaceutical composition comprises pharmaceutically acceptable excipients and/or other pharmaceutically active ingredients.

Said pharmaceutically active ingredient is preferably active in the treatment of pain in a mammal or human subject.

As used herein, a “pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations meet general safety, sterility, pyrogenicity and purity standards required by regulatory offices, such as, for example, EMA or FDA.

In one embodiment, said pharmaceutical composition is administered in a dosage regimen that comprises a therapeutically effective amount of said one or more molecules of the invention.

Pharmaceutical compositions of the invention can be administered in various forms, for example in an injectable, pulverizable or ingestible form, for example via the intramuscular, intravenous, subcutaneous, intradermal, oral, topical, rectal, vaginal, ophthalmic, nasal, transdermal or parenteral route. In one embodiment, said pharmaceutical composition is administered by subcutaneous route.

In one embodiment, said pharmaceutical composition is used in a method involving repeated opioid administration (chronic administration), and in particular repeated administration of said pharmaceutical composition. Repeated administration may last for example at least 4 days, for examples at least 87 days.

In the figures:

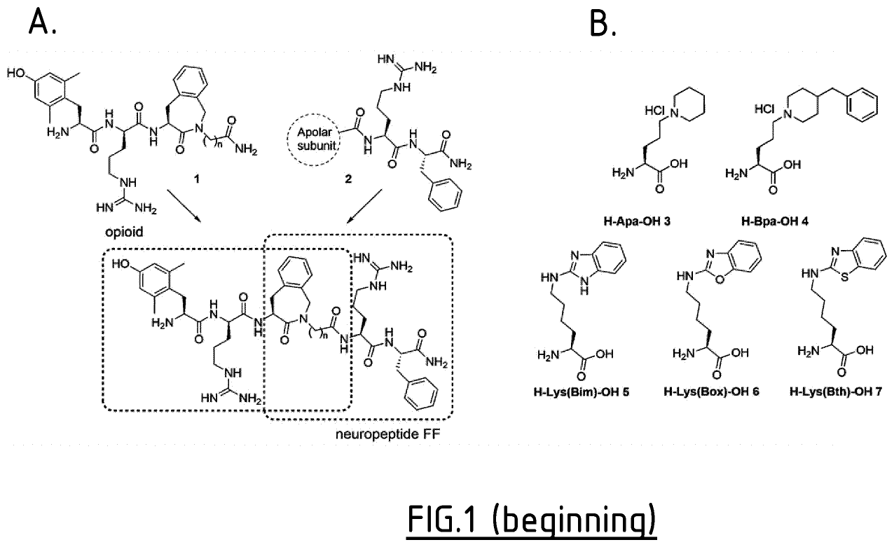

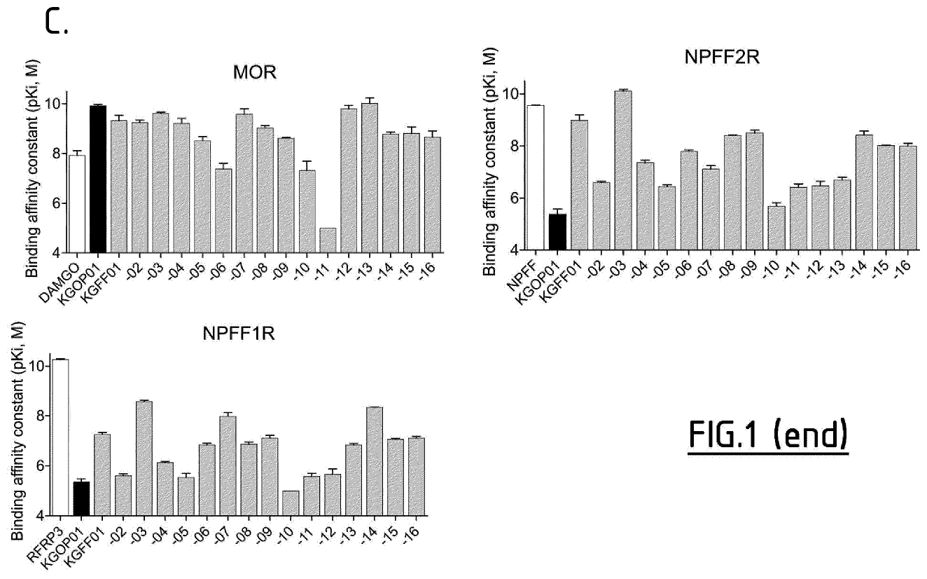

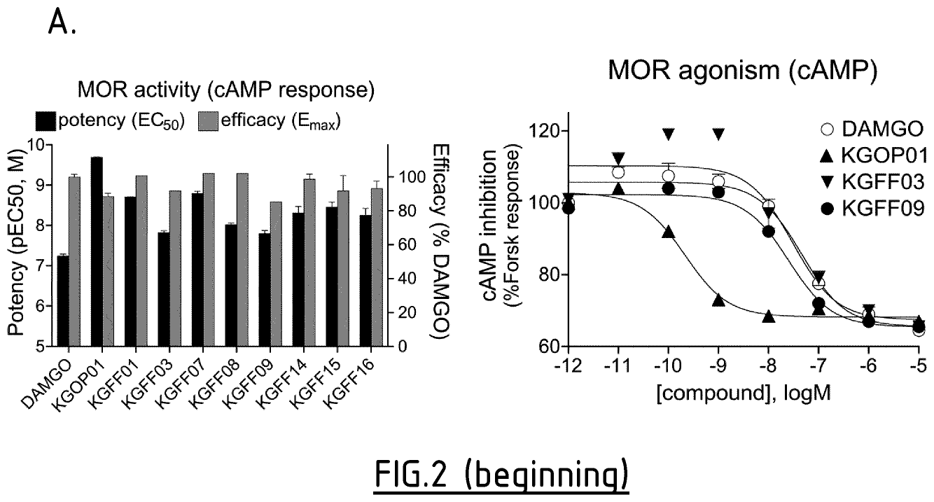

FIG. 1: Design strategy and in vitro screening of KGFF peptidomimetics for affinity at MOPr, NPFF1R and NPFF2R.

A: KGFF compounds combining the opioid ligand KGOP01 and NPFF ligand pharmacophores. From this strategy, 16 compounds were synthesized and screened in vitro for their affinity for the MOPr, NPFF1R and NPFF2R.
B: Chemical diversity at the NPFF ligand pharmacophore with Arg mimetics/Orn derivatives serving as Arg mimetics for the NPFF ligand pharmacophore.
C: Binding affinity constants (pKᵢ values) were determined in radioligand competition binding assays with membranes from CHO cells expressing hMOPr, hNPFF1R or hNPFF2R. Values are mean±SEM of at least 2 independent experiments performed in duplicate.

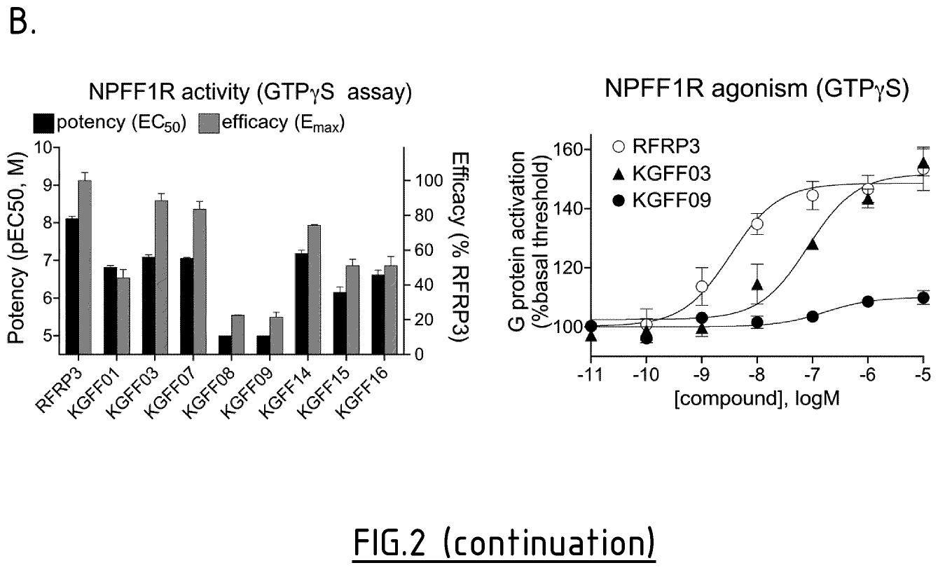

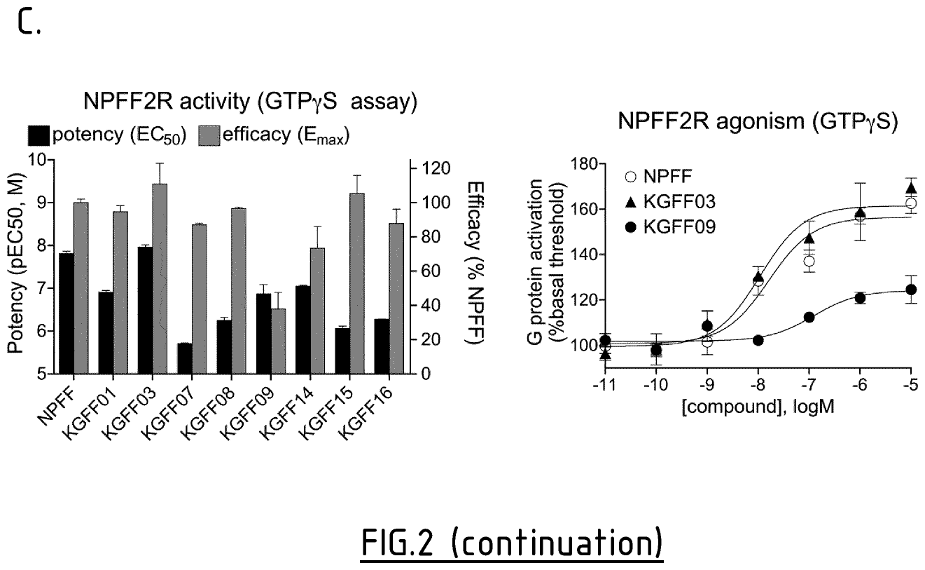

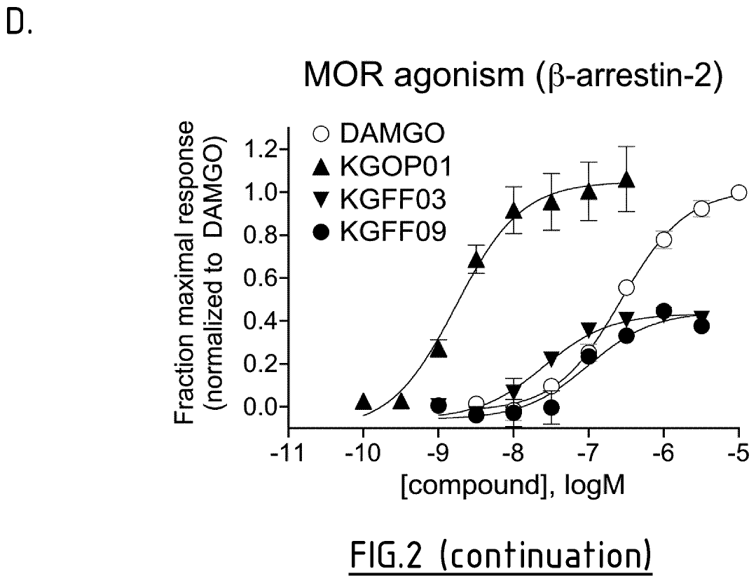

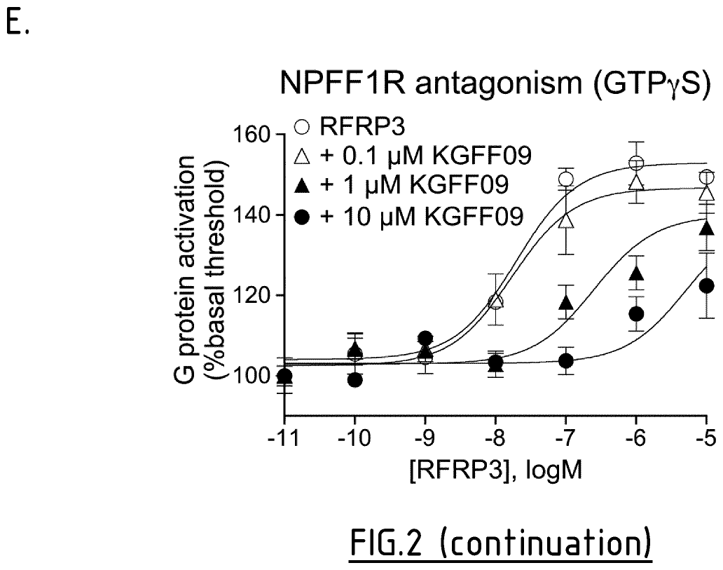

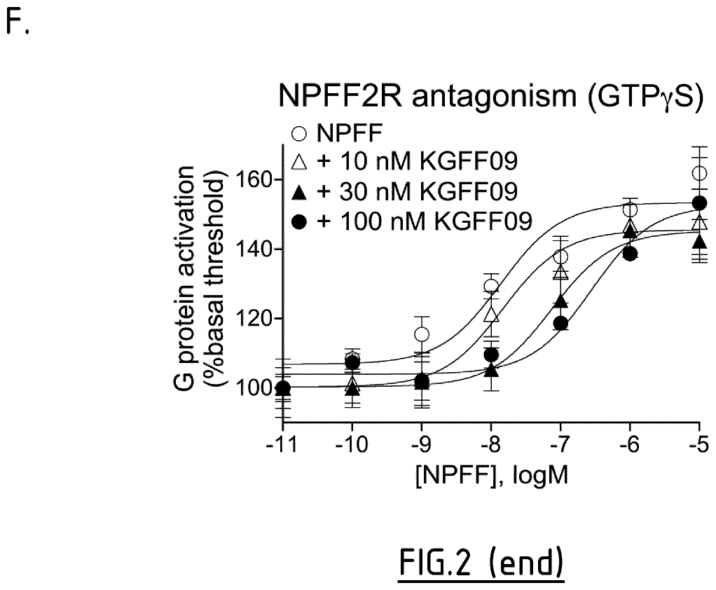

FIG. 2: In vitro functional characterization of KGFF peptidomimetics.

A: Inhibition of forskolin-induced cAMP accumulation in HEK293-Glo20E-hMOPr cells.
B, E: hNPFF1R activation measured by [³⁵S]-GTPγS binding with membranes from CHO-hNPFF1R cells.
C, F: hNPFF2R activation measured by [³⁵S]-GTPγS binding with membranes from CHO-hNPFF2R cells.
D: eYFP-labelled β-arrestin-2 translocation to Rluc-hMOPr in HEK293 cells. Agonist specific BRET1 ratio were determined by subtracting BRET1 ratio of non-activated cells, and normalized to the maximal effect of DAMGO.
(A, B, C) Potency constants (pEC₅₀) and efficacy values (Eₘₐₓ) are shown on left panels and representative experiments for KGOP01, KGFF03 and KGFF09 on right panels. Efficacy values (Eₘₐₓ) are relative to DAMGO (A), RFRP3 (B) or NPFF (C) response. NPFF1R (E) and NPFF2R (F) antagonisms were assessed with RFRP3 or NPFF dose-response curve, respectively, in the presence of increasing KGFF09 concentrations. Values are mean±SEM of at least 2 independent experiments performed in duplicate or triplicate.

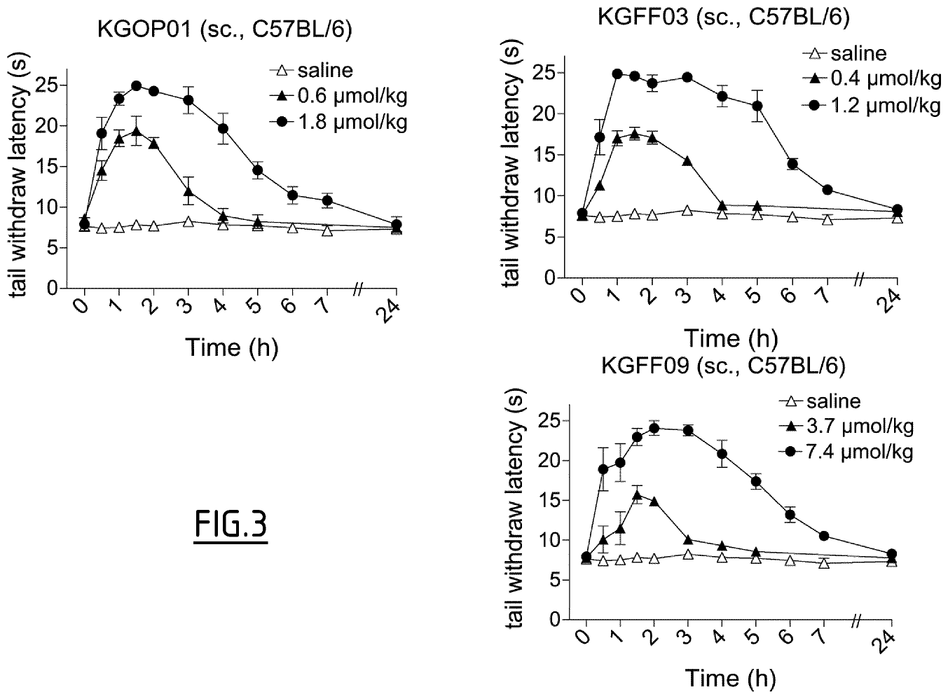

FIG. 3: Antinociceptive effect of KGOP01, KGFF03 and KGFF09 after acute sc. administration to mice. Time- and dose-dependent analgesic effects of KGOP01, KGFF03 and KGFF09 in the tail immersion test after sc. administration in C57BL/6N mice. Withdrawal latencies are expressed in seconds and presented as mean±SEM (n=6-7).

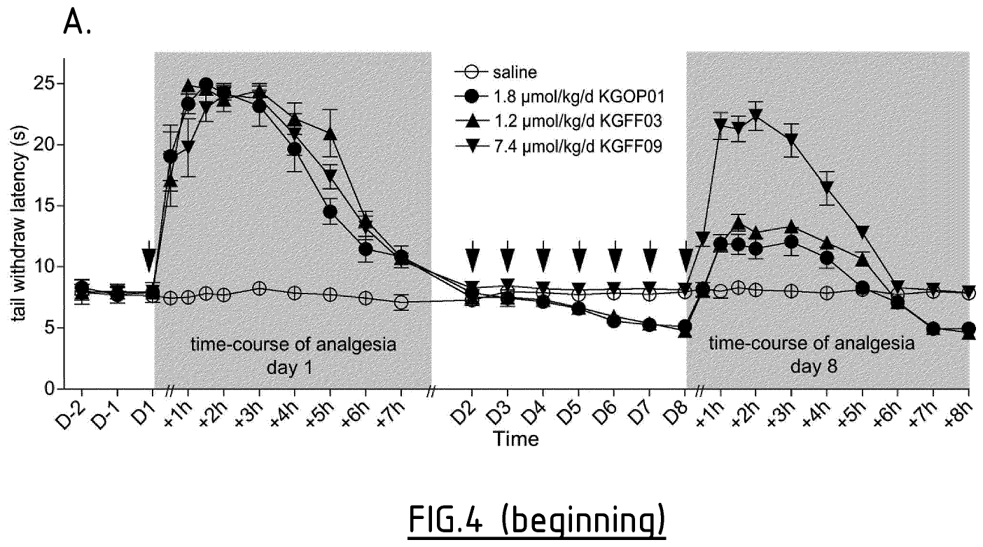

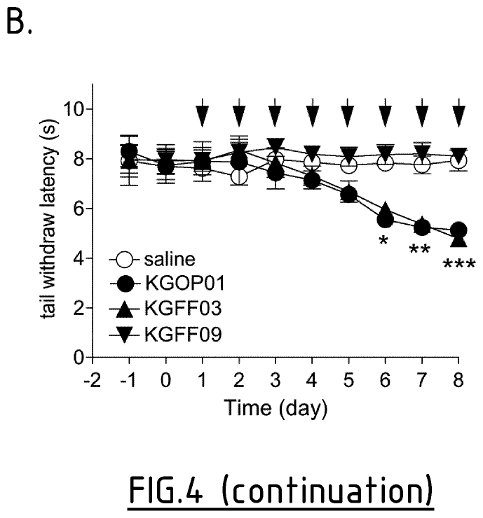

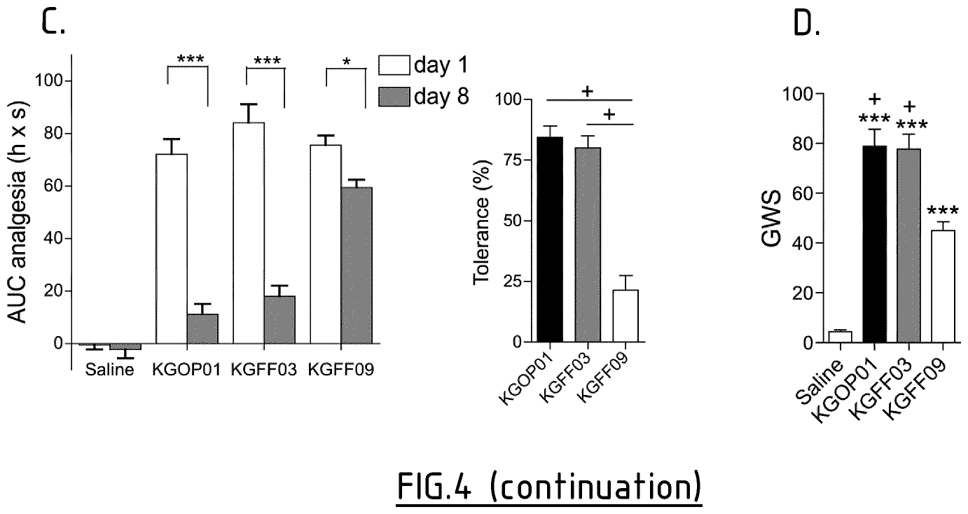

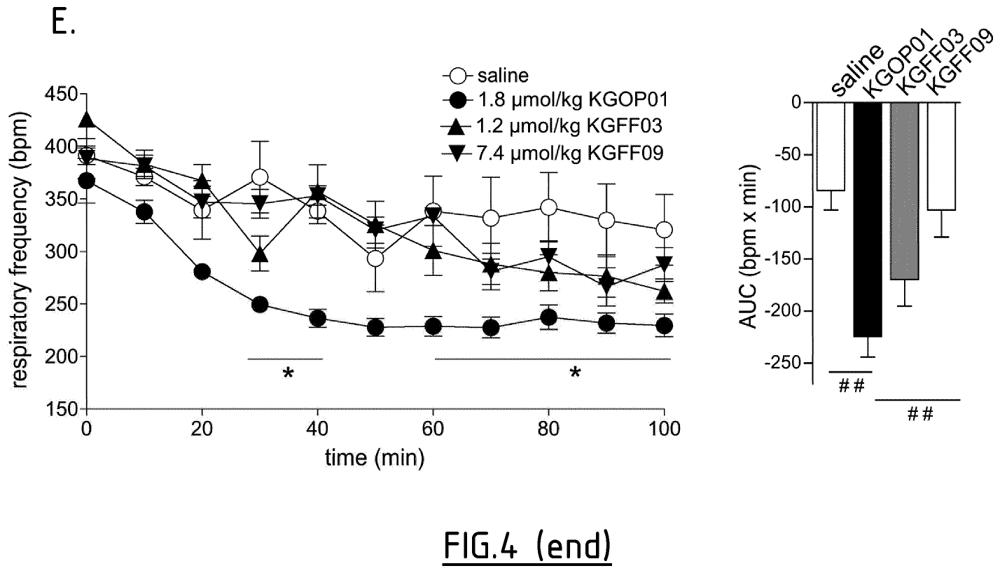

FIG. 4: Adaptive responses induced by KGOP01, KGFF03 and KGFF09 after chronic sc. treatment of mice.

A: Development of hyperalgesia and analgesic tolerance upon chronic treatment with KGOP01, KGFF03 and KGFF09. C57BL/6N mice received daily (d1 to d8) injections of KGOP01 (1.8 μmol/kg, sc.), KGFF03 (1.2 μmol/kg, sc.), KGFF09 (7.4 μmol/kg, sc.) or saline (A, B, C).
B: Development of hyperalgesia upon chronic treatment of C57BL/6N mice with KGOP01, KGFF03 and KGFF09. Basal nociceptive values were measured for two days before drug treatment and once daily before drug administration (d1 to d8), using the tail immersion test. Each day of injection is shown with an arrowhead.
C: Development of analgesic tolerance upon chronic treatment of C57BL/6N mice with KGOP01, KGFF03 and KGFF09. Comparison between groups of area-under-the-curve (AUC) values over the 0-7 h time course is shown on the left panel and development of tolerance (%) at day 8 relative to day 1 on the right panel.
D: Effect of KGOP01, KGFF03 or KGFF09 on naltrexone-precipitated withdrawal signs after chronic treatment of C57BL/6N mice. The different signs of withdrawal were measured over 30 min immediately after naltrexone injection (1 mg/kg, sc.) and a global withdrawal score (GWS) was calculated. KGOP01 (1.8 μmol/kg, sc.), KGFF03 (1.2 μmol/kg, sc.) and KGFF09 (7.4 μmol/kg, sc.) were administered twice daily for 7 days.
E: Effect of KGOP01, KGFF03 and KGFF09 on respiratory frequency after sc. administration to C57BL/6N mice, measured by whole body mouse plethysmography immediately after injection of KGOP01 (1.8 μmol/kg, sc.), KGFF03 (1.2 μmol/kg, sc.), KGFF09 (7.4 μmol/kg, sc.) or saline (sc.). Comparison between groups of respiratory frequency (left panel) and area-under-the curve (AUC, right panel) values over the 100 min kinetics are shown. Data are expressed as mean±SEM. Groups were compared using one-way (C right panel, and D, E right panel) or two-way (B, C left panel, and E left panel) ANOVA with Bonferroni post hoc test *p<0.05, **p<0.01, ***p<0.001 as compared to saline, ##p<0.01 as compared to KGOP01, +p<0.001 as compared to KGFF09.

FIG. 5: KGFF09 induces antinociception with reduced tolerance and anti-hyperalgesia in mice with inflammatory pain.

C57BL/6N mice were injected on day 1 with CFA or saline in the tail, and then daily administered with KGOP01 (1.8 μmol/kg/d, sc.), KGFF03 (1.2 μmol/kg/d, sc.), KGFF09 (7.4 μmol/kg/d, sc.) or saline (sc., CFA/saline and saline/saline).
A: Analgesic tolerance to the thermal antinociceptive effect was measured 2 h after daily sc. administration on days 2-3-5-7 by the tail immersion test. Comparison between groups of the % MPE (left panel) and tolerance (%) at day 7 relative to day 2 (right panel) are shown.
B: Analgesic tolerance to the mechanical antinociceptive effect was measured 2 h after daily sc. injection on days 2-4-6-8 by the tail pressure test. Comparison between groups of percentage of the % MPE (left panel) and tolerance (%) at day 8 relative to day 2 (right panel) are shown.
C, D: Anti-hyperalgesic activity of KGOP01, KGFF03 and KGFF09. Basal nociceptive thresholds were measured by the tail immersion test (C) or tail pressure test (D) once daily before drug administration to visualize CFA-induced pain hypersensitivity.
Data are expressed as mean±SEM, n=8-10. Statistical significance was calculated with two-way ANOVA with Bonferroni post hoc test *p<0.5, **p<0.01, ***p<0.001 vs saline mice, +p<0.5, ++p<0.01, +++p<0.001 vs KGOP01, and with one-way ANOVA with Bonferroni post hoc test ###p<0.001 vs KGFF09.

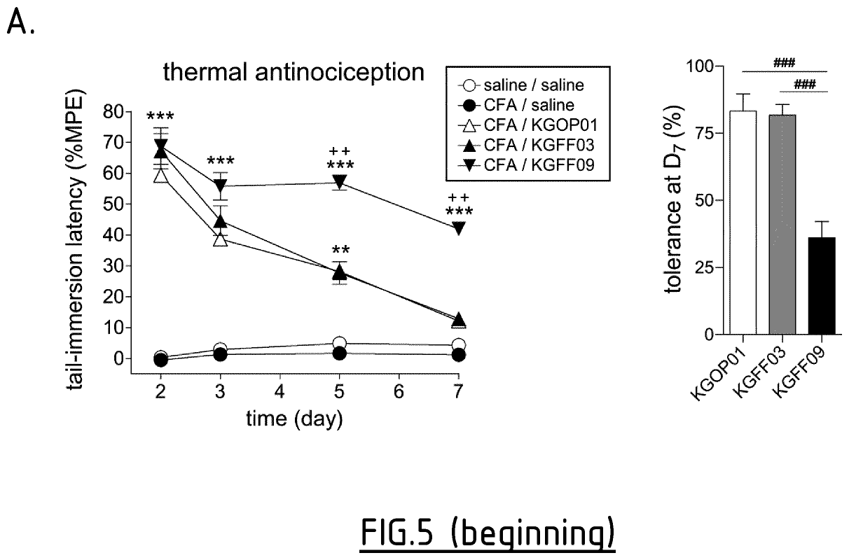

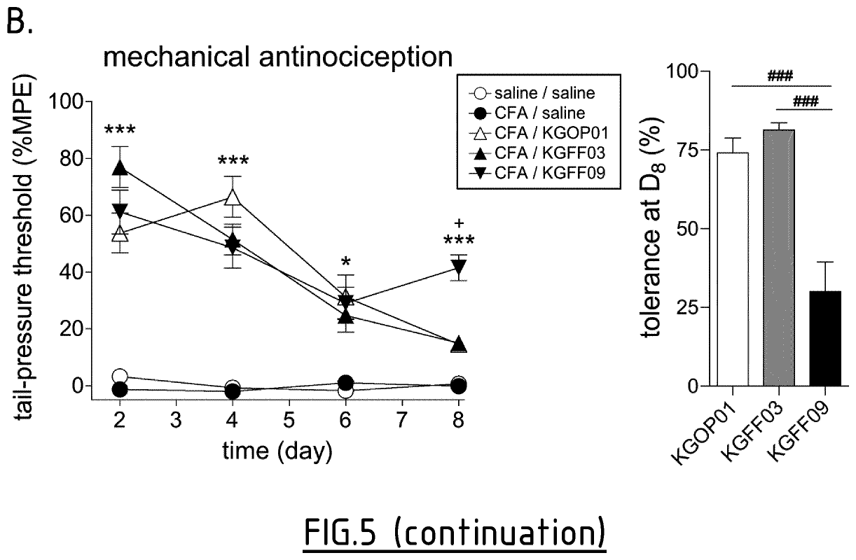

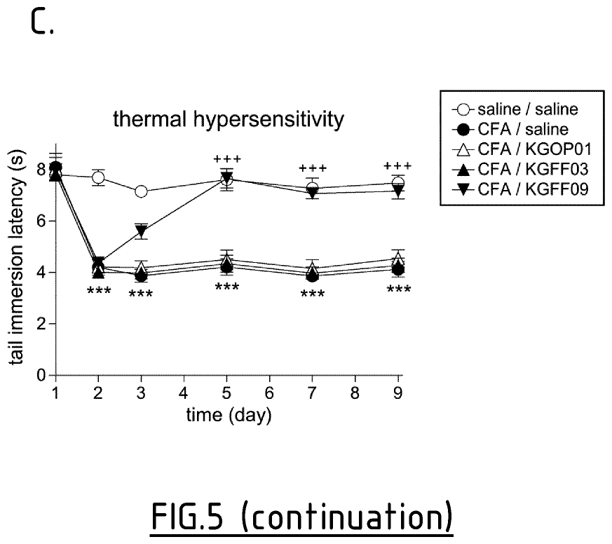

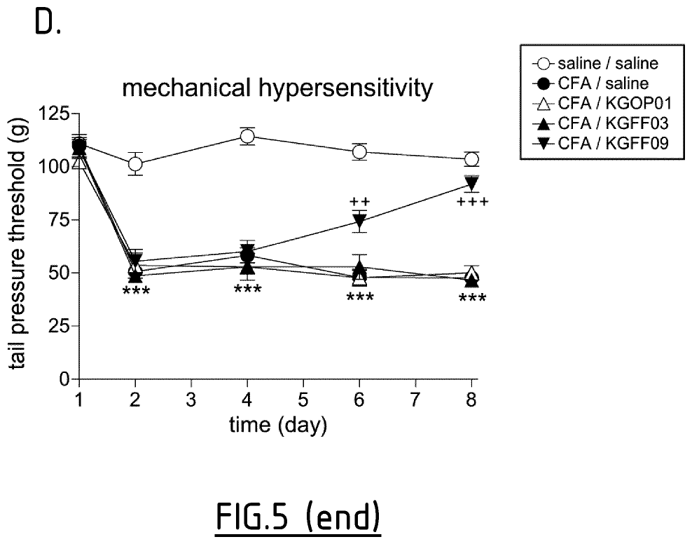

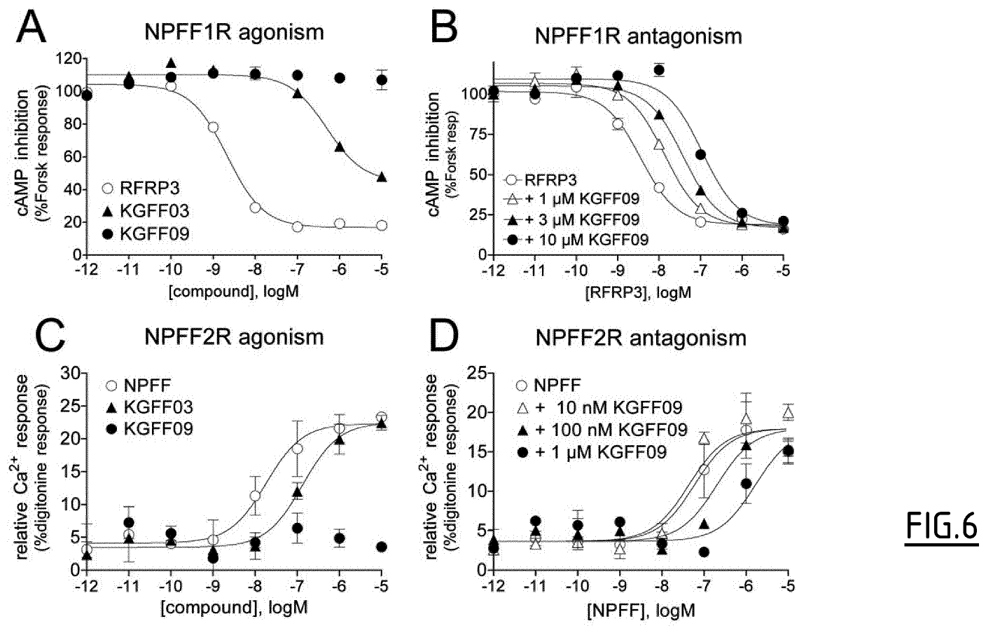

FIG. 6: in vitro characterization of KGFF03 and KGFF09 on NPFFRs.

Agonist (A) or antagonist (B) activity of KGFF03 and KGFF09 measured by inhibition of forskoline-induced cAMP accumulation (A, B) in HEK293-Glo20F cells stably expressing NPFF1R. Data are expressed as percentage of maximal cAMP levels. Agonist (C) or antagonist (D) activity of KGFF03 and KGFF09 measured by Ca²⁺ release (C, D) in HEK293-Glo20F cells stably expressing NPFF2R. Data are expressed as percentage of maximal digitonine-induced Ca²⁺ response, relative to basal. Data are mean±SEM of at least 2 independent experiments performed in duplicate.

FIG. 7: In vitro characterization and selectivity of KGOP01, KGFF03 and KGFF09 for activity at DOPr, DOPr and NOPr.

Agonist (A, B and E) or antagonist (C, D, F and G) activity of KGOP01, KGFF03 and KGFF09 measured by inhibition of forskoline-induced cAMP accumulation in HEK293-Glo20F cells stably expressing human DOPr (A), KOPr (B, C and D) or NOPr (E, F and G). Data are expressed as percentage of maximal cAMP levels and shown as mean±SEM of at least 2 independent experiments performed in duplicate.

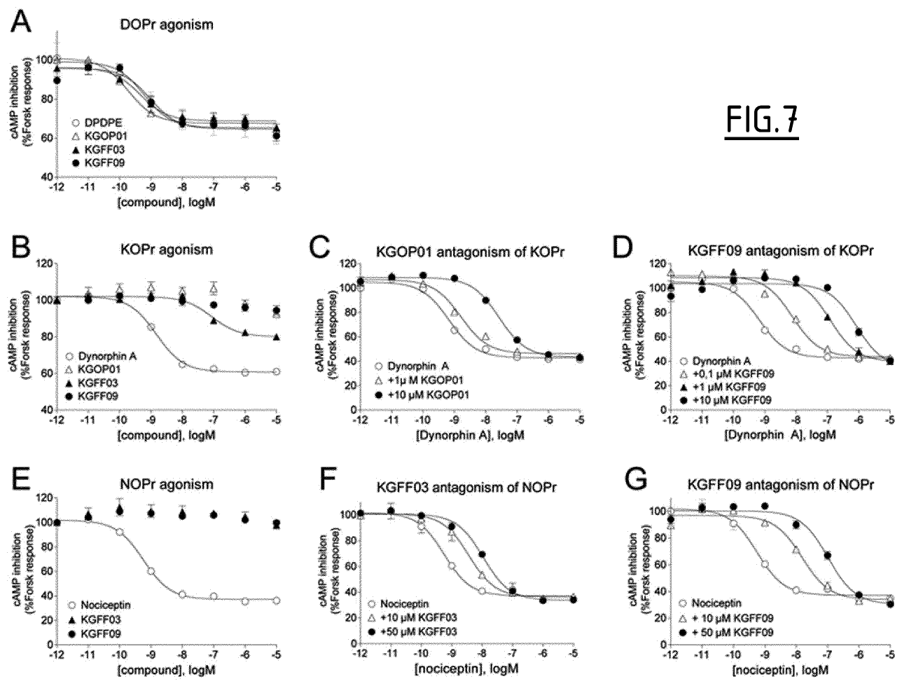

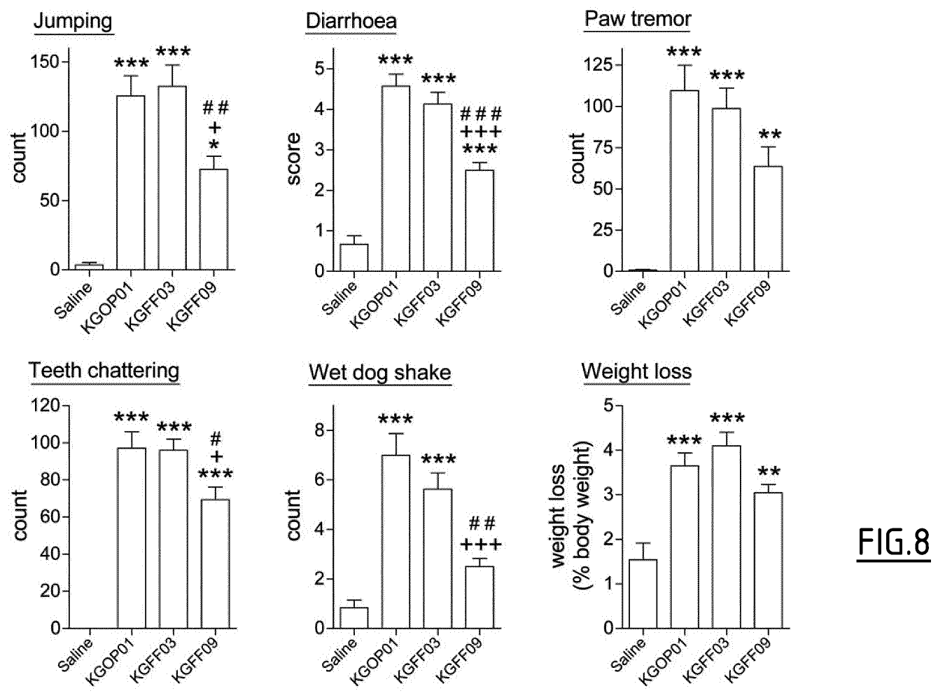

FIG. 8: Effect of KGOP01, KGFF03 or KGFF09 on naltrexone-precipitated withdrawal syndrome in C57BL/6 mice after chronic sc. treatment.

The signs of withdrawal were measured over 30 min immediately after naltrexone sc. injection. Mice were treated with KGOP01 (1.8 μmol/kg, sc.), KGFF03 (1.2 μmol/kg, sc.), KGFF09 (7.4 μmol/kg, sc.) or saline (control) twice daily over a 7-days period. Data are mean±SEM, n=6-8. One way ANOVA with Bonferroni's post hoc test *p<0.05, **p<0.01, ***p<0.001 as compared to saline, +p<0.05, +++p<0.001 as compared to KGOP01 and #p<0.05, ##p<0.01, ###p<0.001 as compared to KGFF03.

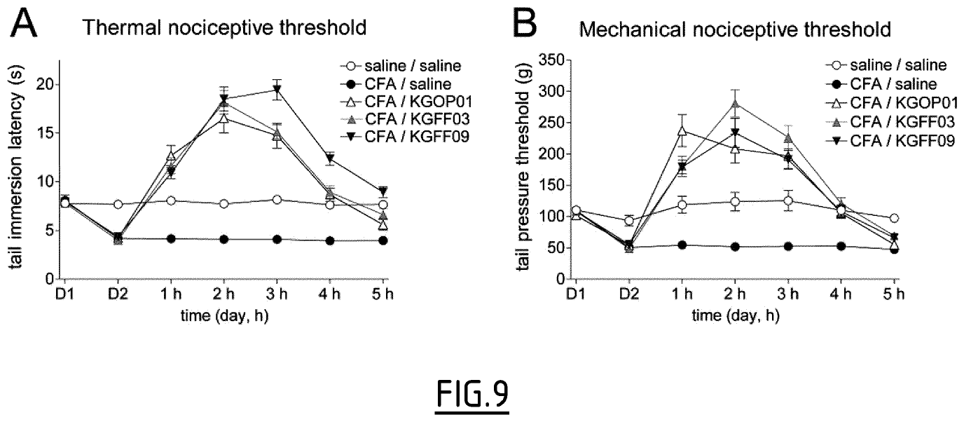

FIG. 9: Acute antinociceptive effect of KGOP01, KGFF03 and KGFF09 in the CFA-induced inflammatory pain model.

Time-dependent antinociceptive effect of KGOP01, KGFF03 and KGFF09 sc. administration in C57BL/6 mice in the tail immersion test (A) and in the tail pressure test (B). KGOP01 (1.8 μmol/kg/d, sc.), KGFF03 (1.2 μmol/kg/d, sc.), KGFF09 (7.4 μmol/kg/d, sc.) or saline (control) were administered 24 h after CFA (or saline) injection in the tail. Data are mean±SEM, n=9-10.

### EXAMPLES

1. Materials and Methods

### 1.1. Chemical Synthesis

### 1.1.1. Peptides KGFF01-KGFF07

Peptides KGFF01-KGFF07 were synthesized manually by standard Fmoc-SPPS on Rink amide AM resin. Standard couplings were performed with 3 equivalents (equiv.) of Fmoc-protected amino acid and 3 equiv. of coupling reagent (HCTU) in 0.4 NMM in DMF during 1.5 h. Fmoc-Aba-β-Ala-OH was coupled in only 1.5 equiv. excess of both Fmoc-dipeptide and coupling reagent for 3 h. Boc-Dmt-OH was coupled using 1.5 equiv. of amino acid and 1.5 equiv. of HOBt/DIC in DMF for 2 h, without addition of base to avoid coupling to the unprotected phenol group. For Fmoc-deprotection, the resin was treated with 20% 4-methylpiperidine in DMF for consecutively 5 and 15 min, or with DBU/Piperidine/DMF 2/2/96 for consecutively 3×30 s, and 7 min. Washing of the resin was performed after every coupling and after deprotection step with DMF (3×), iPrOH or MeOH (3×) and CH₂Cl₂ (3×). Final cleavage and deprotection were done with the cleavage mixture (TFA/TES/H₂O 95:2.5:2.5, TES can be replaced by TIS) during 3 h. After filtration and concentration of TFA, the residue was added to cold ether to precipitate the peptide. The ether phase was decanted and the peptide was dissolved in acetonitrile/H₂O or water and lyophilized to obtain the crude peptides as a powder. The crude peptides were dissolved in H₂O and acetonitrile was added until complete dissolving was observed. This solution was purified by preparative RP-LC (system PLC-A or PLC-B). Fractions containing the product were combined and lyophilized. The final peptides were obtained with a purity >95% as white powders. The compounds were characterized by high-resolution electrospray mass spectroscopy.

### 1.1.2. Peptides KGFF08-KGFF11, DP0001-DP0005, DP0032-DP0035

The couplings of these four peptides were carried out using 3 equiv. of the amino acid with 3 equiv. of DIC/HOBt for 1.5 h in DMF. Coupling of both Fmoc-Aba-β-Ala-OH, Fmoc-Apa-OH and Fmoc-Bpa-OH were performed with only 1.5 equiv. of amino acid and coupling reagent (DIC/HOBt) in DMF during 3 h. Boc-Dmt-OH was coupled with the same number of equivalents, but the reaction mixture was stirred only for 2 h. Further standard SPPS coupling, cleavage and purification were performed as described for peptides KGFF01-KGFF07

### 1.1.3 Peptide KGFF12

The dipeptide Boc-Dmt-D-Arg(Pbf)-OH was first synthesized on 2-chlorotrityl chloride resin, using the same coupling and deprotection conditions as described above. Cleavage was performed with 1% TFA in CH₂Cl₂ to retain the side chain protective group. The solution was then evaporated and the dipeptide (1.1 equiv.) was dissolved in CH₂Cl₂. Two equiv. DIPEA and 1.5 equiv. DIC/HOBt were added and the mixture was stirred for 30 min at 0° C. The free 4-amino-Aba-NH was added and stirred for another 30 min in an ice bath. The reaction was then left on stirring during 16 h at room temperature. After this coupling, the protective groups were removed with the same cleavage cocktail as described for the preparation of peptides KGFF01-KGFF07, and purification was performed analogously.

### 1.1.4. Peptide KGFF13

This synthesis was performed on FMPB-AM resin (4-(4-formyl-3-methoxyphenoxy)butyrylaminomethyl resin). Methylamine hydrochloride (10 equiv.) was coupled to the aldehyde resin by a reductive amination with sodium cyanoborohydride (10 equiv.) in MeOH/DMF for 2.5 h at 80° C. Complete coupling was determined by the DNPH-color test. Further standard SPPS coupling, cleavage and purification were performed as described for peptides KGFF01-KGFF07 (vide infra).

### 1.1.5. Peptides KGFF14-KGFF16

These 3 peptides were synthesized on MBHA-resin. The couplings (Boc-Dmt-OH, Boc-D-Arg(Tos)-OH, Boc-Phe-OH) were performed with 3 equiv. of amino acid and 3 equiv. of coupling reagent (HCTU) in 0.4 NMM in DMF for 1.5 h. Both the arginine mimetics and Boc-Dmt-OH were coupled with DIC/HOBt in DMF with respectively 1.5 (Arg mimetics) and 2 equiv. of amino acid and coupling reagent. Fmoc-Aba-β-Ala-OH was coupled with 1.5 equiv. of HCTU in 0.4 NMM in DMF for 3 h. This Fmoc-group was removed as described above. The Boc-deprotection was performed with 50% TFA in CH₂Cl₂ for 5 and 15 min and washed as previously described. The neutralization step after every Boc-deprotection was realized with a solution of 20% triethylamine in CH₂Cl₂ during 10 min (2 times). Final cleavage was done with liquid HF (10 mL/g resin) and anisole (0.5 mL per 50 mg resin) as scavenger for 1 h at 0° C. After HF distillation, cold ether was added to precipitate the peptides. The peptides were filtered and dissolved in acetic acid/H₂O, and lyophilized. The white powders could be purified by preparative RP-HPLC. Only in case of the paramethoxybenzyl-protected benzoimidazole-containing peptide (KGFF14), an extra step had to be performed to fully cleave the protective group: the peptide was treated with 10% triflic acid (0.5 mL) in TFA (4.5 mL) for 4 h, the solvent was evaporated and the product was purified by preparative RP-HPLC.

### 1.1.2. Peptides DP0007 to DP0031

The couplings of these peptides were carried out using 1.5 equiv. of the amino acid with 3 equiv. of DIC and 5 equiv. of oxyma-pure for 3 h to 4 h in DMF. Further standard SPPS coupling, cleavage and purification were performed as described for peptides KGFF01-KGFF07

### 1.1.7. Peptide DP0032

DP0032 was synthesized using 1.5 eq of amino acid, 3 eq of DIC and HOBt in DMF for 3 to 4 h. Fmoc-Dmt-OH was coupled using 2 eq of amino acid and 3 eq of DIC/Oxyma pure in DMF assisted by micro-waves (75° C. for 30 min). N-terminal guanidylation was performed using 4 eq of N,N′-di-Boc-1H-pyrazole-1-carboxamidine in DMF for 16 h (repeated two times). Further standard SPPS coupling, cleavage and purification were performed as described for peptides KGFF01-KGFF07

### 1.1.8. Peptide DP0035

DP0035 was synthesized using 1.5 eq of amino acid, 3 eq of DIC and HOBt for 3 to 4 h. The N-alkylated glycine residue was introduced following the sub-monomer strategy. After Fmoc-Aba-bAla-OH coupling and Fmoc removal, the N-terminal amine was bromoacylated using 6 eq of bromoacetic acid and 6 eq DIC in DMF for 30 min. The bromide derivative was then displaced using 15 eq of N-Boc-1,4-butanediamine in DMF for 1 h. Finally, Boc-Dmt-OH was coupled with the resulting secondary amine using 3 eq of amino acid and 3 eq of DIC/Oxyma pure in DMF assisted by micro-waves (75° C. for 30 min). Further standard SPPS coupling, cleavage and purification were performed as described for peptides KGFF01-KGFF07.

### 1.2 Peptide Characterization and Synthesis of the Arginine Mimetics and Ornithine Mimetics

### 1.2.1. Materials

Naltrexone hydrochloride, forskolin, 3-isobutyl-1-methylxanthine (IBMX), [D-Ala²,Me-Phe⁴,Gly-ol⁵]enkephalin (DAMGO), probenecid and Complete Freund's Adjuvant (CFA) were purchased from Sigma-Aldrich (Saint Quentin Fallavier, France). Glass bead were purchased from Sigma Aldrich Chemicals (St; Louis, Mo., USA). [D-Pen², D-Pen⁵]enkephalin (DPDPE) and dynorphin were obtained from Abcam (Paris, France), nociception from Polypeptide (Strasbourg, France), morphine hydrochloride from Francopia (Paris, France) and the Fluo-4 acetoxymethyl ester from Molecular Probes (Invitrogen, Cergy Pontoise, France). Human RF-amide peptides were obtained from Genecust (Luxembourg; Kp-10, NPFF, QRFP26 or 26RFa, PrRP-20 and RFRP-3). [¹²⁵I]-1-DMe-NPFF (2200 Ci/mmol) and [³H]-PrRP-20 (150 Ci/mmol) were obtained from Hartmann Analytic (Braunschweig, Germany). [³⁵S]Guanosine 5′-O-[g-thio] triphosphate ([³⁵S]GTPgS; 1250 Ci/mmol), [³H]-diprenorphine (42.3 Ci/mmol), [³H]-nociceptine (114.7 Ci/mmol), [¹²⁵I]-Kp-10 (2200 Ci/mmol) and [¹²⁵I]-QRFP43 (2200 Ci/mmol) were purchased from Perkin Elmer Life and Analytical Sciences (Courtaboeuf, France) and Luciferin from Synchem UG & Co KG (Felsberg, Germany). All other chemicals were of analytical grade and obtained from standard commercial sources.

### 1.2.2. Synthesis and Compound Characterization, General

Thin-layer chromatography (TLC) was performed on glass plates precoated with silica gel 60F254 (Merck, Darmstadt, Germany) using the mentioned solvent systems. Mass Spectrometry (MS) was done on a Q-Tof spectrometer with electrospray ionisation (ESI). Data collection and spectrum analysis was done with Masslynx software. Analytical RP-HPLC was performed using system LC-A (including a Waters 717plus Autosampler, a Waters 1525 Binary HPLC Pump and a Waters 2487 Dual Absorbance Wavelength Detector (Milford, Mass.), with a Grace (Deerfield, Ill.) Vydac RP 018 column (25 cm×4.6 mm×5 m) using UV detection at 215 nm) or system LC-B (including a LC 1200 Agilent with a Zorbax Agilent 018-column (018, 50 mm×2.1 mm; 1.8 μm), using UV detection with DAD scan from 190 nm to 700 nm). For LC-A, the mobile phase is a mixture of water and acetonitrile and contains 0.1% TFA. The used gradient runs from 3 to 100% acetonitrile in 20 minutes at a flow rate of 1 mL/min. For LC-B, the mobile phase is a mixture of water and acetonitrile and contains 0.05% formic acid. The used gradient runs from 2 to 100% acetonitrile in 8 minutes at a flow rate of 0.5 mL/min. Preparative RP-HPLC purification was done on system PLC-A (including a Gilson (Middleton, Wis.) HPLC system with Gilson 322 pumps, controlled by the software package Unipoint, and a reversed phase 018 column (Discovery® BIO SUPELCO Wide Pore 018 column, 25 cm×2.21 cm, 5 mm) with a linear gradient of 1%/min increase of acetonitrile in water (both having 0.1% TFA)) or a system PLC_B (including a Prep Spot II from Armen, and a reversed phase 018 column (Waters® XSelect CSH Prep C18 5 μM 19×150 mm) with a gradient of acetonitrile in water (having 0.1% TFA), running from 5% to 100% in 30 to 50 minutes). After purification, the purity of all compounds was evaluated as being more than 95% by analytical RP-HPLC. All fractions were lyophilized using a Flexy-Dry lyophilizer (FTS Systems, Warminster, Pa.) or a Lyovapor L-200 (Buchi). ¹H and ¹³C NMR spectra were recorded at 500 and 125 MHz on a Bruker Avance II 500 or at 400 and 100.62 MHz on a Bruker Avance 400 (Bruker Corp, Billerica, Mass.). Tetramethylsilane (TMS) or residual solvent signals are used as internal standard. The solvent used is mentioned in all cases, and the abbreviations used are as follows: s (singlet), d (doublet), dd (double doublet), t (triplet), q (quadruplet) and m (multiplet).
1.2.3. Peptide characterization
H-Dmt-D-Arg-Aba-Gly-Arg-Phe-NH₂ (KGFF01). Preparative RP-HPLC (PLC-A) yielded the desired compound (white powder, 15%). HPLC (LC-A): t_{R}=9.9 min. TLC Rf 0.69 (EBAW). HRMS (ESP⁺) found m/z 884.4915 [M+H]⁺, [C₄₄H₆₁N₁₃O₇+H⁺] required 884.4890.
H-Dmt-D-Arg-Aba-Gly-Arg-Phe-OH (KGFF02). Preparative RP-HPLC (PLC-A) yielded the desired compound (white powder, 20%). HPLC (LC-A): t_{R}=10.4 min. TLC Rf 0.65 (EBAW). HRMS (ESP⁺) found m/z 885.4764 [M+H]⁺, [C₄₄H₆₀N₁₂O₈+H⁺] required 885.4730.
H-Dmt-D-Arg-Aba-b-Ala-Arg-Phe-NH₂ (KGFF03). Preparative RP-HPLC (PLC-A) yielded the desired compound (white powder, 18%). HPLC (LC-A): t_{R}=10.0 min. TLC Rf 0.67 (EBAW). HRMS (ESP⁺) found m/z 898.5076 [M+H⁺], [C₄₅H₆₃N₁₃O₇+H⁺] required 898.5046.
H-Dmt-D-Arg-Aba-Gly-Orn-Phe-NH₂ (KGFF04). Preparative RP-HPLC (PLC-A) yielded the desired compound (white powder, 17%). HPLC (LC-A): t_{R}=9.9 min. TLC Rf 0.67 (EBAW). HRMS(ESP⁺) found m/z 842.4694 [M+H]⁺, [C₄₃H₅₉N₁₁O₇+H⁺] required 842.4672.
H-Dmt-D-Arg-Phe-Orn-Phe-NH₂ (KGFF05). Preparative RP-HPLC (PLC-A) yielded the desired compound (white powder, 27%). HPLC (LC-A): t_{R}=10.1 min. TLC Rf 0.67 (EBAW). HRMS (ESP⁺) found m/z 773.4477 [M+H]⁺, [C₄₀H₅₆N₁₀O₆+H⁺] required 773.4457.
H-Dmt-Arg-Phe-NH₂ (KGFF06). Preparative RP-HPLC (PLC-A) yielded the desired compound (white powder, 44%). HPLC (LC-A): t_{R}=10.1 min. TLC Rf 0.68 (EBAW). HRMS (ESP⁺) found m/z 512.2994 [M+H⁺], [C₂₆H₃₇N₇O₄+H⁺] required 512.2980.
H-Dmt-D-Arg-Phe-NH₂ (KGFF07). Preparative RP-HPLC (PLC-A) yielded the desired compound (white powder, 63%). HPLC (LC-A): t_{R}=8.9 min. TLC Rf 0.71 (EBAW). HRMS (ESP⁺) found m/z 512.2977 [M+H⁺], [C₂₆H₃₇N₇O₄+H⁺] required 512.2980.
H-Dmt-D-Arg-Aba-b-Ala-Apa-Phe-NH₂ (KGFF08). Preparative RP-HPLC (PLC-A) yielded the desired compound (white powder, 20%). HPLC (LC-A): t_{R}=10.2 min. TLC Rf 0.46 (EBAW). HRMS (ESP⁺) found m/z 924.5476 [M+H⁺], [C₄₉H₆₉N₁₁O₇+H⁺] required 924.5454.
H-Dmt-D-Arg-Aba-b-Ala-Bpa-Phe-NH₂ (KGFF09). Preparative RP-HPLC (PLC-A) yielded the desired compound (white powder, 17%). HPLC (LC-A): t_{R}=12.0 min. TLC Rf 0.67 (EBAW). HRMS (ESP⁺) found m/z 1014.5931 [M+H⁺], [C₅₆H₇₅N₁₁O₇+H⁺] required 1014.5923.
H-Dmt-Apa-Phe-NH₂ (KGFF10). Preparative RP-HPLC (PLC-A) yielded the desired powder, (25%). HPLC (LC-A): t_{R}=10.0 min. TLC Rf 0.43 (EBAW). HRMS (ESP⁺) found m/z [C₃₀H₄₃N₅O₄+H⁺] required 538.3388.
H-Dmt-Bpa-Phe-NH₂ (KGFF11). Preparative RP-HPLC (PLC-A) yielded the desired powder, (9%). HPLC (LC-A): t_{R}=12.0 min. TLC Rf 0.70 (EBAW). HRMS (ESP⁺) found m/z [C₃₇H₄₉N₅O₄+H⁺] required 628.3857.
H-Dmt-D-Arg-Aba-NH (KGFF12). Preparative RP-HPLC (PLC-A) yielded the desired powder, (15%). HPLC (LC-A): t_{R}=9.0 min. TLC Rf 0.66 (EBAW). HRMS (ESP⁺) found m/z [C₂₇H₃₇N₇O₄+H⁺] required 524.2980.
H-Dmt-D-Arg-Phe-NHMe (KGFF13). Preparative RP-HPLC (PLC-A) yielded the desired compound (white powder, 8%). HPLC (LC-A): t_{R}=9.0 min. TLC Rf 0.66 (EBAW). HRMS (ESP⁺) found m/z 526.3140 [M+H⁺], [C₃₁H₃₉F₆N₇O₆+H⁺] required 526.3136.
H-Dmt-D-Arg-Aba-b-Ala-Lys(Bim)-Phe-NH₂ (KGFF14). (PLC-A) Preparative RP-HPLC yielded the desired compound (white powder, 22%). HPLC (LC-A): t_{R}=11.2 min. TLC Rf 0.69 (EBAW). HRMS (ESP⁺) found m/z 986.5317 [M+H⁺], [C₅₂H₆₇N₁₃O₇+H⁺] required 986.5359.
H-Dmt-D-Arg-Aba-b-Ala-Lys(Box)-Phe-NH₂ (KGFF15). (PLC-A) Preparative RP-HPLC yielded the desired compound (white powder, 43%). HPLC (LC-A): t_{R}=11.3 min. TLC Rf 0.74 (EBAW). HRMS (ESP⁺) found m/z 987.5257 [M+H⁺], [C₅₂H₆₆N₁₂O₈+H⁺] required 987.5200.
H-Dmt-D-Arg-Aba-b-Ala-Lys(Bth)-Phe-NH₂ (KGFF16). (PLC-A) Preparative RP-HPLC yielded the desired compound (white powder, 39%). HPLC (LC-A): t_{R}=11.4 min. TLC Rf 0.70 (EBAW). HRMS (ESP⁺) found m/z 1003.4985 [M+H⁺], [C₅₂H₆₆N₁₂O₇S+H⁺] required 1003.4970.
H-Dmt-D-Arg-Aba-β-Ala-Bpa-Val-NH₂ (DP0001). Preparative RP-HPLC (PLC-A) yielded the desired compound (white powder, 8%). HPLC (LC-B): t_{R}=4.16 min. HRMS (ESP⁺) found m/z 965.5859 [M+H⁺], [C₅₂H₇₅N₁₁O₇+H⁺] required 965.5851.
H-Dmt-D-Arg-Aba-β-Ala-Bpa-Ile-NH₂ (DP0002). Preparative RP-HPLC (PLC-A) yielded the desired compound (white powder, 15%). HPLC (LC-B): t_{R}=4.29 min. HRMS (ESP⁺) found m/z 979.6017 [M+H⁺], [C₅₃H₇₇N₁₁O₇+H⁺] required 979.6007.
H-Dmt-D-Arg-Aba-β-Ala-Bpa-Leu-NH₂ (DP0003). Preparative RP-HPLC (PLC-A) yielded the desired compound (white powder, 24%). HPLC (LC-B): t_{R}=4.32 min. HRMS (ESP⁺) found m/z 979.6019 [M+H⁺], [C₅₃H₇₇N₁₁O₇+H⁺] required 979.6007.
H-Dmt-D-Arg-Aba-β-Ala-Bpa-Tyr-NH₂ (DP0004). Preparative RP-HPLC (PLC-A) yielded the desired compound (white powder, 28%). HPLC (LC-B): t_{R}=4.18 min. HRMS (ESP⁺) found m/z 1029.5819 [M+H⁺], [C₅₆H₇₅N₁₁O₇+H⁺] required 1029.5800.
H-Dmt-D-Arg-Aba-β-Ala-Bpa-Trp-NH₂ (DP0005). Preparative RP-HPLC (PLC-A) yielded the desired compound (white powder, 15%). HPLC (LC-B): t_{R}=4.49 min. HRMS (ESP⁺) found m/z 1052.5938 [M+H⁺], [C₅₈H₇₆N₁₂O₇+H⁺] required 1052.5960.
H-Dmt-N(Me)-D-Ala-Aba-β-Ala-Bpa-Phe-NH₂ (DP0007). Preparative RP-HPLC (PLC-B) yielded the desired compound (white powder, 2%). HPLC (LC-B): t_{R}=6.12 min. HRMS (ESP⁺) found m/z 942.5409 [M+H⁺], [C₅₄H₇₀N₈O₇+H⁺] required 942.5367.
H-Dmt-D-Pro-Aba-β-Ala-Bpa-Phe-NH₂ (DP0008). Preparative RP-HPLC (PLC-B) yielded the desired compound (white powder, 9%). HPLC (LC-B): t_{R}=5.80 min. HRMS (ESP⁺) found m/z 954.5410 [M+H⁺], [C₅₅H₇₀N₈O₇+H⁺] required 954.5367.
H-Dmt-D-Bpa-Aba-β-Ala-Bpa-Phe-NH₂ (DP0009). Preparative RP-HPLC (PLC-B) yielded the desired compound (white powder, 4%). HPLC (LC-B): t_{R}=5.94 min. HRMS (ESP⁺) found m/z 1129.6759 [M+H⁺], [C₆₇H₈₇N₉O₇+H⁺] required 1129.6728.
H-Dmt-D-Arg-1AnaGly-Bpa-Phe-NH₂ (DP0012). Preparative RP-HPLC (PLC-B) yielded the desired compound (white powder, 1%). HPLC (LC-B): t_{R}=4.58 min. HRMS (ESP⁺) found m/z 1049.5877 [M+H⁺], [C₅₉H₇₅N₁₁O₇+H⁺] required 1049.5851.
H-Dmt-D-Arg-Phe-N(Me)-β-Ala-Bpa-Phe-NH₂ (DP0013). Preparative RP-HPLC (PLC-B) yielded the desired compound (white powder, 5%). HPLC (LC-BA): t_{R}=4.34 min. HRMS (ESP⁺) found m/z 1015.6013 [M+H⁺], [C₅₆H₇₇N₁₁O₇+H⁺] required 1015.6007.
H-Dmt-N(Me)-D-Ala-1AnaGly-Bpa-Phe-NH₂ (DP0014). Preparative RP-HPLC (PLC-B) yielded the desired compound (white powder, 3%). HPLC (LC-B): t_{R}=5.34 min. HRMS (ESP⁺) found m/z 978.5375 [M+H⁺], [C₅₇H₇₀N₈O₇+H⁺] required 978.5367.
H-Dmt-D-Arg-Aba-β-Ala-Bpa-D-Phe-NH₂ (DP0015). Preparative RP-HPLC (PLC-B) yielded the desired compound (white powder, 1%). HPLC (LC-B): t_{R}=4.33 min. HRMS (ESP⁺) found m/z 1013.5872 [M+H⁺], [C₅₆H₇₅N₁₁O₇+H⁺] required 1013.5851.
H-Dmt-D-Arg-Aba-β-Ala-Bpa-Phe-β-Ala-NH₂ (DP0016). Preparative RP-HPLC (PLC-B) yielded the desired compound (white powder, 1%). HPLC (LC-B): t_{R}=4.26 min. HRMS (ESP⁺) found m/z 1084.6235 [M+H⁺], [C₅₉H₈₀N₁₂O₇+H⁺] required 1084.6222.
H-Dmt-N(Me)-D-Ala-AbaGABA-Bpa-Phe-NH₂ (DP0017). Preparative RP-HPLC (PLC-B) yielded the desired compound (white powder, 6%). HPLC (LC-B): t_{R}=5.03 min. HRMS (ESP⁺) found m/z 956.5498 [M+H⁺], [C₅₅H₇₂N₈O₇+H⁺] required 956.5524.
H-Dmt-D-Arg-AbaGABA-Bpa-Phe-NH₂ (DP0018). Preparative RP-HPLC (PLC-B) yielded the desired compound (white powder, 2%). HPLC (LC-B): t_{R}=4.36 min. HRMS (ESP⁺) found m/z 1027.5985 [M+H⁺], [C₅₅H₇₇N₁₁O₇+H⁺] required 1027.6007.
H-Dmt-D-Arg-Phe-β-Ala-Bpa-Phe-NH₂ (DP0019). Preparative RP-HPLC (PLC-B) yielded the desired compound (white powder, 2%). HPLC (LC-B): t_{R}=4.30 min. HRMS (ESP⁺) found m/z 1001.5834 [M+H⁺], [C₅₅H₇₅N₁₁O₇+H⁺] required 1001.5851.
H-Dmt-D-Arg-AbaGABA-Bpa-Val-NH₂ (DP0020). Preparative RP-HPLC (PLC-B) yielded the desired compound (white powder, 3%). HPLC (LC-B): t_{R}=4.12 min. HRMS (ESP⁺) found m/z 979.5993 [M+H⁺], [C₅₃H₇₇N₁₁O₇+H⁺] required 979.6007.
H-Dmt-D-Arg-1AnaGly-Bpa-Val-NH₂ (DP0021). Preparative RP-HPLC (PLC-B) yielded the desired compound (white powder, 4%). HPLC (LC-B): t_{R}=4.41 min. HRMS (ESP⁺) found m/z 1001.5853 [M+H⁺], [C₅₅H₇₅N₁₁O₇+H⁺] required 1001.5851.
H-Dmt-D-Arg-AbaGABA-Bpa-Trp-NH₂ (DP0022). Preparative RP-HPLC (PLC-B) yielded the desired compound (white powder, 1%). HPLC (LC-B): t_{R}=4.42 min. HRMS (ESP⁺) found m/z 1066.6101 [M+H⁺], [C₅₉H₇₈N₁₂O₇+H⁺] required 1066.6116.
H-Dmt-D-Arg-1AnaGly-Bpa-Trp-NH₂ (DP0023). Preparative RP-HPLC (PLC-B) yielded the desired compound (white powder, 1%). HPLC (LC-B): t_{R}=4.65 min. HRMS (ESP⁺) found m/z 1088.5926 [M+H⁺], [C₆₁H₇₆N₁₁O₇+H⁺] required 1088.5960.
H-Dmt-D-Arg-Phe-N(Me)Gly-Bpa-Phe-NH₂ (DP0024). Preparative RP-HPLC (PLC-B) yielded the desired compound (white powder, 27%). HPLC (LC-B): t_{R}=4.37 min. HRMS (ESP⁺) found m/z 1001.5822 [M+H⁺], [C₅₅H₇₅N₁₁O₇+H⁺] required 1001.5851.
H-Dmt-D-Arg-Phe-N(Me)Gly-Bpa-Val-NH₂ (DP0025). Preparative RP-HPLC (PLC-B) yielded the desired compound (white powder, 22%). HPLC (LC-B): t_{R}=4.09 min. HRMS (ESP⁺) found m/z 953.5872 [M+H⁺], [C₅₁H₇₅N₁₁O₇+H⁺] required 953.5851.
H-Dmt-D-Arg-Aba-β-Ala-THIQ-Phe-NH₂ (DP0026). Preparative RP-HPLC (PLC-B) yielded the desired compound (white powder, 4%). HPLC (LC-B): t_{R}=3.97 min. HRMS (ESP⁺) found m/z 971.5374 [M+H⁺], [C₅₃H₆₉N₁₁O₇+H⁺] required 971.5381.
H-Dmt-D-Arg-Aba-β-Ala-D-Bpa-Phe-NH₂ (DP0027). Preparative RP-HPLC (PLC-B) yielded the desired compound (white powder, 2%). HPLC (LC-B): t_{R}=4.37 min. HRMS (ESP⁺) found m/z 1013.5862 [M+H⁺], [C₅₆H₇₅N₁₁O₇+H⁺] required 1013.5851.
H-Dmt-D-Orn-Aba-β-Ala-Bpa-Phe-NH₂ (DP0028). Preparative RP-HPLC (PLC-B) yielded the desired compound (white powder, 11%). HPLC (LC-B): t_{R}=4.27 min. HRMS (ESP⁺) found m/z 971.5647 [M+H⁺], [C₅₅H₇₃N₉O₇+H⁺] required 971.5633.
H-Dmt-D-Lys-Aba-β-Ala-Bpa-Phe-NH₂ (DP0029). Preparative RP-HPLC (PLC-B) yielded the desired compound (white powder, 9%). HPLC (LC-B): t_{R}=4.30 min. HRMS (ESP⁺) found m/z 985.5805 [M+H⁺], [C₅₆H₇₅N₉O₇+H⁺] required 985.5789.
H-Dmt-D-Arg-Phe-N(Me)-D-Ala-Bpa-Phe-NH₂ (DP0030). Preparative RP-HPLC (PLC-B) yielded the desired compound (white powder, 11%). HPLC (LC-B): t_{R}=4.51 min. HRMS (ESP⁺) found m/z 1015.6033 [M+H⁺], [C₅₆H₇₇N₁₁O₇+H⁺] required 1015.6007.
7-OH-Tic-D-Arg-Aba-β-Ala-Bpa-Phe-NH₂ (DP0031). Preparative RP-HPLC (PLC-B) yielded the desired compound (white powder, 1%). HPLC (LC-B): t_{R}=4.35 min. HRMS (ESP⁺) found m/z 997.5539 [M+H⁺], [C₅₅H₇₁N₁₁O₇+H⁺] required 997.5538.
Guanidyl-Dmt-D-Arg-Aba-β-Ala-Bpa-Phe-NH₂ (DP0032). Preparative RP-HPLC (PLC-A) yielded the desired compound (white powder, 10%). HPLC (LC-B): t_{R}=4.51 min. HRMS (ESP⁺) found m/z 1055.6045 [M+H⁺], [C₅₇H₇₇N₁₃O₇+H⁺] required 1055.6069.
H-Dmt-D-hArg-Aba-β-Ala-Bpa-Phe-NH₂ (DP0033). Preparative RP-HPLC (PLC-A) yielded the desired compound (white powder, 14%). HPLC (LC-B): t_{R}=4.48 min. HRMS (ESP⁺) found m/z 1027.6010 [M+H⁺], [C₅₇H₇₇N₁₁O₇+H⁺] required 1027.6007.
H-Dmt-D-Lys(Nic)-Aba-β-Ala-Bpa-Phe-NH₂(DP0034). Preparative RP-HPLC (PLC-A) yielded the desired compound (white powder, 21%). HPLC (LC-B): t_{R}=5.05 min. HRMS (ESP⁺) found m/z 1090.6024 [M+H⁺], [C₆₂H₇₈N₁₀O₈+H⁺] required 1090.6004.
H-Dmt-Nlys-Aba-β-Ala-Bpa-Phe-NH₂ (DP0035). Preparative RP-HPLC (PLC-A) yielded the desired compound (white powder, 19%). HPLC (LC-B): t_{R}=4.46 min. HRMS (ESP⁺) found m/z 985.5801 [M+H⁺], [C₅₆H₇₅N₉O₇+H⁺] required 985.5789.

### 1.2.4. Synthesis of the Arginine Mimetics

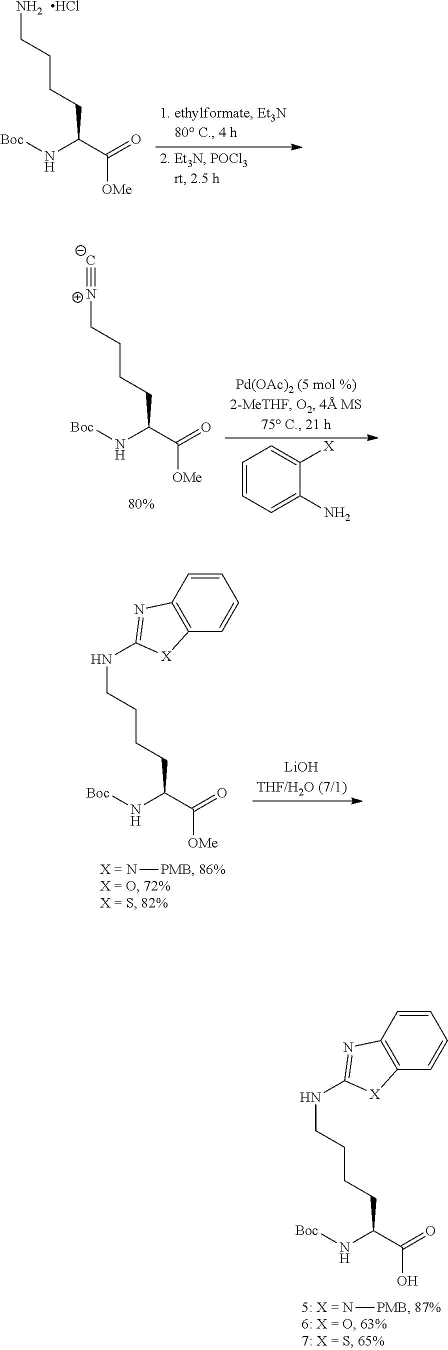

### 1.2.5. Synthesis of Boc-Lys(NC)-OMe

Boc-Lys-OMe hydrochloride (3.00 g, 10.1 mmol, 1.0 equiv.) was dissolved in ethylformate (28.8 mL, 35.5 mmol, 35 equiv.). To this solution, triethylamine (1.4 mL, 10.1 mmol, 1.0 equiv.) was added and stirred for 4 h at 80° C. After cooling down to room temperature, the mixture was evaporated in vacuo. The crude formamide was obtained as a white solid and used without further purification. The formamide was dissolved in dry CH2Cl2 (20 mL) and triethylamine (7.0 mL, 50.5 mmol, 5.0 equiv.) was added to this solution. The solution was flushed with argon and cooled to 0° C. Subsequently, phosphoryl chloride (1.4 mL, 15.2 mmol, 1.5 equiv.) was added dropwise while stirring. After the addition, the ice bath was removed and the mixture was stirred for an additional 2.5 h at room temperature. The mixture was poured in cold water (30 mL) and extracted with CH2Cl2 (3×30 mL). The combined organic layers were washed with water and brine (2×30 mL) and dried over MgSO4. After concentration, the product was purified by flash chromatography using heptane/EtOAc (1/1) as eluent. (S)-methyl-2-((tert-butoxycarbonyl)amino)-6-isocyanohexanoate was obtained in 80% yield. Yield: 80% (yellow oil, 3.64 g); Formula: C₁₃H₂₂N₂O₄; MW=270.32 g/mol; TLC Rf: =0.59 (EtOAc/heptane 1/1); 1H-NMR (CDCl3, 400 MHz) in ppm d: 1.44 (s, 9H), 1.48-1.55 (m, 2H), 1.63-1.72 (m, 3H), 1.82-1.89 (m, 1H), 3.39 (t, J=6.0 Hz, 2H), 3.75 (s, 3H), 4.31 (br s, 1H), 5.04 (br s, 1H); 13C-NMR (CDCl3, 100 MHz) in ppm d: 22.1, 28.3, 28.5, 32.0, 41.3 (t, J=6.4 Hz), 52.4, 53.0, 80.1, 155.4, 156.4, 172.9.

### 1.2.6. Synthesis of Boc-Lys(Bim-PMB)-OMe

A 25 mL Kjeldahl flask was flame dried under vacuum and refilled with argon. Subsequently, the vial was charged with Pd(OAc)₂ (11 mg, 0.05 mmol, 0.05 equiv.), N-(p-methoxybenzyl)-o-phenylenediamine (228 mg, 1.00 mmol, 1.0 equiv.) and 4 Å molecular sieves (300 mg). The flask was equipped with a reflux condenser, evacuated and back filled with 02 (three times). (S)-methyl-2-((tert-butoxycarbonyl)amino)-6-isocyanohexanoate (324 mg, 1.20 mmol, 1.2 equiv.) was dissolved in 2-MeTHF (1 mL) in a separate vial under argon. This mixture was added to the Kjeldahl flask followed by 2-MeTHF (1.5 mL). The reaction mixture was stirred at 75° C. under 02 atmosphere for 21 h. After cooling down to room temperature, the solution was filtered through celite using EtOAc (50 mL) and concentrated in vacuo. The product was purified by automated flash chromatography applying a heptane/EtOAc gradient (from 100% heptane to 10% EtOAc, 25 mL/min). Methyl-2-((tert-butoxycarbonyl)amino)-6-((1-(4-methoxybenzyl)-1H-benzoimidazol-2-yl)amino)hexanoate was obtained in 86% (425 mg) yield. Yield: 86% (brown oil, 425 mg); Formula: C₂₇H₃₆N₄O₅; MW=496.60 g/mol; TLC Rf=0.61 (acetone/heptane 1/4); HPLC: t_{R}=14.8 min; HRMS (ES⁺): found 497.2744, calculated 497.2758 [M+H]⁺; 1H-NMR (CDCl₃, 400 MHz) in ppm δ 1.23-1.40 (m, 4H), 1.43 (s, 9H), 1.57-1.67 (m, 4H), 1.75-1.81 (m, 2H), 3.47 (q, J=6.5 Hz, 2H), 3.70 (s, 3H), 3.78 (s, 3H), 4.01 (br s, 1H), 4.25 (br s, 1H), 5.02 (br s, 3H), 6.85-6.88 (m, 2H), 7.01-7.14 (m, 5H), 7.51 (d, J=7.8 Hz, 1H); 13C-NMR (CDCl₃, 100 MHz) in ppm δ: 22.5, 28.2, 29.1, 32.2, 42.9, 45.0, 52.0, 53.2, 55.1, 79.7, 107.1, 114.4, 116.2, 119.5, 121.1, 127.3, 127.7, 134.6, 142.1, 154.2, 155.3, 159.3, 173.1.

### 1.2.7. Synthesis of Boc-Lys(Box)-OMe

A 25 mL Kjeldahl flask was flame dried under vacuum and refilled with argon. Subsequently, the vial was charged with Pd(OAc)₂ (11 mg, 0.05 mmol, 0.05 equiv.), 2-aminophenol (109 mg, 1.0 mmol, 1.0 equiv.) and 4 Å molecular sieves (300 mg). The flask was equipped with a reflux condenser, evacuated and back filled with 02 (three times). (S)-methyl-2-((tert-butoxycarbonyl)amino)-6-isocyanohexanoate (324 mg, 1.20 mmol, 1.2 equiv.) was dissolved in 2-MeTHF (1 mL) in a separate vial under argon. This mixture was added to the Kjeldahl flask followed by 2-MeTHF (1.5 mL). The reaction mixture was stirred at 75° C. under 02 atmosphere for 21 h. After cooling down to room temperature, the solution was filtered through celite using EtOAc (50 mL) and concentrated in vacuo. The product was purified by an automated flash chromatography applying a heptane/EtOAc gradient (from 100% heptane to 10% EtOAc, 25 mL/min). Tert-butyl 6-(benzoxazol-2-ylamino)-2-((methoxycarbonyl)amino)hexanoate was obtained in 72% (270 mg) yield. Yield: 72% (brown solid, 270 mg); Formula: C₁₉H₂₇N₃O₅; MW=377.43 g/mol; TLC Rf=0.32 (EtOAc/heptane 1/1); HPLC t_{R}=13.0 min; HRMS (ES⁺): found 378.2000, calculated 378.2023 [M+H]⁺; 1H-NMR (CDCl3,400 MHz) in ppm δ=1.44-1.51 (s, 11H), 1.64-1.90 (m, 4H), 3.48 (m, 2H), 4.73 (s, 3H), 4.32 (br s, 1H), 5.08 (s, 2H), 7.02 (td, J=7.8, 1.0 Hz, 1H), 7.15 (td, J=7.7, 0.8 Hz, 1H), 7.22 (d, J=7.9 Hz, 1H), 7.36 (d, J=7.7 Hz, 1H); 13C-NMR (CDCl3, 100 MHz) in ppm δ 22.6, 28.5, 29.2, 32.8, 43.0, 52.5, 53.2, 80.2, 108.8, 116.5, 121.0, 124.0, 143.2, 148.7, 155.7, 162.2, 173.3.

### 1.2.7. Synthesis of Boc-Lys(Bth)-OMe

A 25 mL Kjeldahl flask was flame dried under vacuum and refilled with argon. Subsequently, the vial was charged with Pd(OAc)₂ (11 mg, 0.05 mmol, 0.05 equiv.), 2-aminothiophenol (1070, 1.00 mmol, 1.0 equiv.) and 4 Å molecular sieves (300 mg). The flask was equipped with a reflux condenser, evacuated and back filled with 02 (three times). (S)-methyl-2-((tert-butoxycarbonyl)amino)-6-isocyanohexanoate (324 mg, 1.20 mmol, 1.2 equiv.) was dissolved in 2-MeTHF (1 mL) in a separate vial under argon. This mixture was added to the Kjeldahl flask followed by 2-MeTHF (1.5 mL). The reaction mixture was stirred at 75° C. under 02 atmosphere for 21 h. After cooling down to room temperature, the solution was filtered through celite using EtOAc (50 mL) and concentrated in vacuo. The product was purified by an automated flash chromatography applying a heptane/EtOAc gradient (from 100% heptane to 10% EtOAc, 25 mL/min). Tert-butyl 6-(benzothiazol-2-ylamino)-2-((methoxycarbonyl)amino)hexanoate was obtained in 80% yield. Yield: 80% (white solid, 316 mg); Formula: C₁₉H₂₇N₃O₄S; MW: 393.50 g/mol; TLC Rf: =0.61 (EtOAc/heptane 1/2); HPLC: t_{R}=13.1 min; HRMS (ES⁺): found 394.1779, calculated 394.1795 [M+H]⁺; 1H-NMR (CDCl₃, 400 MHz) in ppm δ 1.44-1.48 (m, 10H), 1.50-1.67 (m, 3H), 1.84-1.89 (m, 1H), 3.44 (t, J=6.8 Hz, 2H), 3.73 (s, 3H), 4.32 (br s, 1H), 5.07 (br s, 1H), 5.41 (br s, 1H), 7.07 (td, J=7.6, 0.9 Hz, 1H), 7.26-7.30 (m, 1H), 7.53 (d, J=7.8 Hz, 1H), 7.57 (d, J=7.8 Hz, 1H); 13C-NMR (CDCl₃, 100 MHz) in ppm δ: 22.8, 28.5, 29.1, 31.0, 32.8, 45.3, 52.5, 53.2, 80.2, 119.1, 120.9, 121.7, 126.1, 130.6, 152.7, 167.4, 173.3.

### 1.2.8. Boc-Lys(Bim-PMB)-OH

Boc-Lys(Bim,PMB)-OMe (250 mg, 0.50 mmol) was dissolved in a mixture of THF/H₂O (7:1, total 3.2 mL). Lithium hydroxide monohydrate (148 mg, 3.52 mmol, 7 equiv.) was added and continuously stirred for 16 h at room temperature. The reaction mixture was concentrated by evaporation and resuspended in H₂O (10 mL). The aqueous phase was washed with CH2Cl2 (2×5 mL) and carefully acidified to pH=3, as indicated by pH paper, with 1N HCl. The aqueous layer was extracted with CH2Cl2 (4×10 mL). The combined organic layers were collected, washed with brine (1×20 mL) and dried over MgSO4, filtered and concentrated to obtain the corresponding carboxylic acid as a pink solid in 87% yield. The building block was used in Boc-based SPPS without further purification. Yield: 87% (211 mg); HPLC: t_{R}=14.0 min; HRMS: (ES⁺): found 483.2580, calculated 493.2602 [M+H]⁺.

### 1.2.9. Boc-Lys(Box)-OH

Boc-Lys(Box)-OMe (190 mg, 0.50 mmol) was dissolved in a mixture of THF/H₂O (7:1, total 3.2 mL). Lithium hydroxide monohydrate (148 mg, 3.52 mmol, 7 equiv.) was added and continuously stirred for 16 h at room temperature. The reaction mixture was concentrated by evaporation and resuspended in H₂O (10 mL). The aqueous phase was washed with CH2Cl2 (2×5 mL) and carefully acidified to pH=3, as indicated by pH paper, with 1N HCl. The aqueous layer was extracted with CH2Cl2 (4×10 mL). The combined organic layers were collected and washed with brine (1×20 mL) and dried over MgSO4, filtered and concentrated to obtain the corresponding carboxylic acid as a pink solid in 74% yield (135 mg). The building block was used in Boc-based SPPS without further purification. Yield: 74% (135 mg); HPLC: t_{R}=11.8 min; HRMS (ES⁺): found 364.1867, calculated 364.1867 [M+H]⁺.

### 1.2.10. Boc-Lys(Bth)-OH

Boc-Lys(Bth)-OMe (198 mg, 0.50 mmol) was dissolved in a mixture of THF/H₂O (7:1, total 3.2 mL). Lithium hydroxide monohydrate (148 mg, 3.52 mmol, 7 equiv.) was added and continuously stirred for 16 h at room temperature. The reaction mixture was concentrated by evaporation and resuspended in H₂O (10 mL). The aqueous phase was washed with CH2Cl2 (2×5 mL) and carefully acidified to pH=3, as indicated by pH paper, with 1N HCl. The aqueous layer was extracted with CH2Cl2 (4×10 mL). The combined organic layers were collected and washed with brine (1×20 mL) and dried over MgSO4, filtered and concentrated to obtain the corresponding carboxylic acid as a pink solid in 72% yield. The building block was used in Boc-based SPPS without further purification. Yield: 72% (13 8 mg); HPLC: t_{R}=12.1 min; HRMS (ES⁺): found 380.1617, calculated 380.1638 [M+H]⁺.
1.2.11. Chiral Derivatization of Boc-Lys(Bim,PMB)-OH, Boc-Lys(Box)-OH and Boc-Lys(Bth)-OH with Marfey's Reagent (FDAA)
The chiral derivatization was performed starting from the N-Boc-deprotected substrates. Compounds (10 mg) are treated with a cocktail of TFA/CH₂Cl₂ (1:1) for 1 h and concentrated. The crude was redissolved in H₂O with a minimal amount of AcN and lyophilized to obtain the deprotected mimetics as white to off-white solids in quantitative yields. The samples were analyzed via LC/MS and used without further purification.
Subsequently, the enantiomeric purity was checked via chiral derivatization with Marfey's reagent (FDAA). For this, a stock solution of 20.0 mM FDAA in acetone was prepared (5.44 mg FDAA/1 mL acetone). The analyte (1 mg) was dissolved in 1 mL 1 M NaHCO₃. Two equiv. of the stock solution were added to 100 μL of the analyte solution and the mixture was incubated at 40° C. overnight. After quenching with 100 μL of a 1 M HCl solution, the sample was diluted to 1 mL with water and analyzed by LC-MS. Integration of the peak area (340 nm) gave an estimate of the enantiomeric excess. The derivatized product was obtained as a single peak, indicating that no epimerization took place during the synthesis.
Further analysis of the enantiomeric excess was performed by coupling of Boc-Lys(Bim-PMB)-OH to HCl.H-Ala-OMe by use of EDC/HOAt (1.6 equiv.) to yield the dipeptide. Analysis via HPLC showed only one single peak and NMR did not show any trace of the other diastereomer.
1.2.12. Synthesis of 4-amino-tetrahydro-aminobenzazepinon (Aba)-NH
N-Boc-ortho-aminomethyl-L-Phe (507 mg, 1.72 mmol, 1 equiv.), was submitted to an intramolecular cyclization [1]. The product was dissolved in 172 mL of CH2Cl2 (10 mM) and EDC.HCl (495 mg, 2.58 mmol, 1.5 equiv.), Et₃N (601 μL, 4.31 mmol, 2.5 equiv.) and HOBt.H₂O (396 mg, 2.58 mmol, 1.5 equiv.) were added. The reaction was stirred for 16 h and then the organic phase was washed with 20% citric acid and sat. NaHCO₃-solution. The solvent was dried with MgSO4 and evaporated. The product was purified by flash chromatography using ethyl acetate/petroleum ether (4/6) to give a white solid in 19% yield. Yield: 19% (92.4 mg); Formula: C₁₅H₂₀N₂O₃; MW=276.15 g/mol; TLC Rf=0.34 (EtOAc/petroleum ether4/6); HPLC t_{R}=13.9 min; MS (ES⁺): 277 [M+H]⁺, 299 [M+Na]⁺, 177 [M-Boc]⁺; Melting interval 149.0-150.5° C.; 1H-NMR (CDCl₃, 500 MHz) in ppm □: 1.46 (9H, s, Boc), 2.97 (1H, dd, ²J=16.7 Hz, ³J=13.0 Hz, H_{□}), 3.41 (1H, dd, ²J=16.9 Hz, ³J=3.2 Hz, H_{□′}) 3.96 (1H, dd, ²J=16.7 Hz, ³J=7.1 Hz, H_{□}), 4.85 (1H, dd, ²J=16.5 Hz, ³J=3.8 Hz, H_{□′}), 5.86 (1H, d, ²J=5.8 Hz, NH), 7.01 (1H, d, ³J=7.5 Hz, CH arom.), 7.10 (2H, m, CH arom.), 7.19 (1H, m, CH arom.); 13C-NMR (CDCl3, 125 MHz) in ppm δ: 28.6 (Boc), 37.2 (η-CH2), 46.0 (η-CH2), 49.4 (η-CH), 79.9 (Cq Boc), 126.5 (CH arom.), 128.0 (CH arom.), 128.4 (CH arom.), 131.3 (CH arom.), 134.2 (Cq arom.), 135.7 (Cq arom.), 155.4 (C═O Boc), 174.4 (C═O Aba).
The product could be deprotected with 50% TFA in CH2Cl2 for 2 h and directly coupled to the dipeptide.

### 1.2.13. Synthesis of Fmoc-1-AnaGly-OH

Synthesis of Fmoc-1-AnaGly-OH was performed using the procedure described in the literature (Van der Poorten et al. ACS Med. Chem. Lett. (2017) 8, 11, 1177-82)

### 1.2.14. Synthesis of AbaGABA

Synthesis of Phth-Phe-OH

To a 250 mL round bottom flask, L-phenylalanine (7.72 g, 46.7 mmol) and phthalic anhydride (6.92 g, 46.7 mmol) were added. The solids were then carefully heated up to 145° C. (not above to avoid racemization) and mechanically stirred. Upon heating, the solids started to melt and a brown solid was formed after 1 h indicating completion. The residue was dissolved in hot MeOH (70 mL) stirred and filtered. Cold water (50 mL) was then slowly added to allow crystallization. Pure Phth-Phe-OH was obtained as white crystals after filtration and drying (12.54 g, 91%).

Formula: C17H13NO4; MW: 295.29 g/mol; TLC: Rf=0.43 (CH2Cl2/MeOH 95:5+1% AcOH); HPLC: t_{R}=2.5 min; LC-MS (ES+): [M−COOH]⁺=249.91 Da, [M+H]⁺=295.90 Da; 1H NMR: (500 MHz, 298 K, CDCl3): δ (ppm) 7.75 (m, 2H, arom. H Phth), 7.70 (m, 2H, arom. H Phth), 7.13 (m, 5H, arom. H Phe), 5.22 (t, 1H, H_{α}, J=8.11), 3.61 (dd, 2H, CH_{β}, J=8.60 Hz, J=1.21 Hz).

Synthesis of Phth-Phe-GABA-OEt

Phth-Phe-OH (3.02 g, 10.2 mmol), ethyl 4-aminobutanoate (1.88 g, 11.2 mmol), TBTU (9.75 g, 30.6 mmol) and DCM (50 mL) were added to a 100 mL round bottom flask. TEA (4.25 mL, 30.6 mmol) was then added giving a yellow solution. The pH was monitored and adjusted to 9 until reaction completion by means of TEA addition. After 3 h, the reaction mixture was concentrated and the residue dissolved in ethyl acetate, washed with 1M HCl (3 times), saturated NaHCO₃ solution (3 times) and brine (2 times). The resulting organic phase was dried with MgSO₄, filtered and concentrated under reduced pressure. The crude compound was finally purified by column chromatography using ethyl acetate/petroleum ether as eluent yielding the desired dipeptide (3.02 g, 73%).

Formula: C23H24N2O5; MW: 408.45 g/mol; HPLC: t_{R}=2.7 min; LC-MS (ES+): [M+H]+=408.90, [M+Na]⁺=430.87 Da; HRMS: Calculated [M+H]+=409.1763, mass found [M+H]+=409.1755; TLC: Rf=0.39 (PE/EtOAc 1:1); 1H NMR (500 MHz, 298 K, CDCl3): δ (ppm) 7.79 (m, 2H, arom. H), 7.70 (m, 2H, arom. H), 7.13 (m, 5H, arom. H Phe), 5.10 (dd, 1H, H_{α} Phe, J=11.54 Hz, J=5.40 Hz), 3.98 (m, 2H, —OCH₂CH₃, J=7.16 Hz, J=0.96 Hz), 3.60 (dd, 1H, H_{β} Phe, J=14.2 Hz, J=11.2 Hz)), 3.52 (dd, 1H, H_{β}′ Phe, J=14.2 Hz, J=5.6 Hz), 3.35 (m, 2H, 2H_{γ} GABA), 2.37 (t, 2H, 2H_{α} GABA, J=6.80 Hz), 1.81 (q, 2H, 2H_{β} GABA, J=6.57 Hz), 1.18 (t, 3H, —OCH₂CH₃, J=7.16 Hz); 13C NMR (126 MHz, 295 K, CDCl3): δ (ppm) 174.04 (C═O ester), 168.67 (C═O amide bond), 168.15 (C═O amide Phth), 137.13 (CH arom. Phe), 134.32 (CH arom. phth), 131.74 (Cq arom. Phth), 129.02 (CH arom. Phe), 128.77 (CH arom. Phe), 127.04 (CH arom. Phe), 123.59 (CH arom. Phth), 60.74 (—OCH₂CH₃), 55.96 (CH, Phe), 39.84 (CH_{2γ} GABA), 34.82 (CH_{2β} Phe), 32.11 (CH_{2α} GABA), 24.71 (CH_{2β} GABA) and 14.33 (—OCH₂CH₃).

Synthesis of Phth-Aba-GABA-OEt

To a 250 mL two-necked round bottom flask equipped with a Dean-Stark apparatus, phosphorus pentoxide (12 g, 42.4 mmol) and 85% phosphoric acid (2.73 mL, 40.5 mmol) in acetic acid (60 mL) and benzene (90 mL) were added to give a yellow suspension. The mixture was refluxed for 1 h at 115° C. under stirring and Ar atmosphere. Then Phth-Phe-GABA-OEt (1.5 g, 3.67 mmol) and 1,3,5-trioxane (2.26 g, 25.1 mmol) were added. The mixture was refluxed at 115° C. for 3 h while 1,3,5-trioxane (2.26 g, 25.1 mmol) was added every 30 min. The reaction mixture was cooled to rt and concentrated under reduced pressure giving an orange oil. The residue was dissolved in ethyl acetate and washed with 1M HCl (3 times), saturated NaHCO₃ solution (six times), and once with brine. The resulting organic phase was dried with MgSO4, filtered and concentrated under reduced pressure giving an orange oil. The crude was finally purified by flash chromatography using a Interchim 80 g silica column and petroleum ether/ethyl acetate as eluent (gradient from 20 to 60% ethyl acetate in 25 min) yielding the desired product (0.722 g, 47%).

Formula: C24H24N2O5; MW: 420.47 g/mol; HPLC: t_{R}=2.9 min; LC-MS (ES+): [M+Na]+=442.94 Da, [M+H]+=420.27 Da; HRMS: Calculated [M+H]+=421.1758 Da, mass found [M+H]+=421.1732 Da; TLC: Rf=0.26, EtOAc/PE (1:1); 1H NMR (500 MHz, 298 K, CDCl3): δ (ppm) 7.88 (m, 2H, arom. Phth), 7.75 (m, 2H, arom. Phth), 7.29 (m, 4H, arom. Aba), 5.40 (dd, 1H, H_{α} Aba, J=13.04 Hz, J=4.70 Hz), 4.73 (d, 1H, H_{ε} Aba, J=15.60 Hz), 4.60 (d, 1H, H_{ε}′ Aba, J=15.81 Hz, 65.21 Hz), 4.12 (q, 2H, —OCH₂CH₃, J=7.05 Hz), 3.58 (m, 2H, 2H_{γ} GABA), 3.15 (dd, 2H, 2H_{β} Aba, J=15.71 Hz, J=4.59 Hz), 2.28 (t, 2H, 2H_{α} GABA, J=7.07 Hz), 1.91 (m, 2H, 2H_{β} GABA), 1.24 (t, 3H, —OCH₂CH₃, J=7.05); 13C NMR (126 MHz, 298 K, CDCl3): δ (ppm) 173.34 (C═O ester), 168.54 (C═O amide bond Aba), 168.22 (C═O Phth), 136.16 (Cq arom. Aba), 135.68 (Cq arom. Aba), 134.33 (CH arom. Phth), 132.27 (Cq arom. Phth), 130.30 (CH arom. Aba), 129.30 (CH arom. Aba), 128.78 (CH arom. Aba), 128.44 (CH arom. Aba), 126.97 (CH arom. Aba), 123.70 (CH arom. Phth), 60.60 (—OCH₂CH₃), 51.48 (CH_{α} Aba), 51.37 (CH_{2ε} Aba), 49.02 (CH_{2γ} GABA), 34.30 (CH_{2β} Aba), 31.42 (CH_{2α} GABA), 23.26 (CH_{2β} GABA), 14.41 (—OCH₂CH₃).

Synthesis of Phth-Aba-GA BA-OH

To a 100 mL round bottom flask, Phth-Aba-GABA-OEt (0.722 g, 1.72 mmol), 1M HCl (10 mL) and acetone (10 mL) were added. The reaction mixture was heated at reflux (90° C.) for 16 h under Ar atmosphere. The reaction mixture was concentrated under reduced pressure yielding the desired product as a yellow solid (0.692 g, quantitative) which was used without further purification.

Formula: C22H20N2O5; MW: 392.14 g/mol; HPLC: t_{R}=2.4 min; LC-MS (ES+): [M+H]+=392.80 Da; HRMS: Calculated [M+Na]+=415.1264 Da, mass found [M+Na]+=415.1274 Da; 1H NMR (500 MHz, 298 K, CDCl3): δ (ppm) 7.93 (m, 2H, arom. Phth), 7.89 (m, 2H, arom. Phth), 7.36 (m, 1H, arom. Aba), 7.28 (m, 3H, arom. Aba), 5.28 (dd, 1H, H_{α} Aba, J=12.38, Hz, J=5.27 Hz), 4.84 (d, 1H, H_{ε} Aba, J=15.90 Hz), 4.52 (d, 1H, H_{ε}′ Aba, J=15.81 Hz), 3.94 (dd, 1H, H_{γ} GABA, J=15.92 Hz, J=12.50 Hz), 3.50 (m, 1H, H_{γ}′ GABA), 3.28 (dd, 2H, 2H_{β} Aba, J=16.00 Hz, J=5.25 Hz), 2.10 (t, 2H, 2H_{α} GABA, J=7.70 Hz), 1.69 (m, 2H, 2H_{β} GABA); 13C NMR (126 MHz, 298 K, CDCl3): δ (ppm) 207.24 (C═O—COOH), 174.62 (C═O amide bond), 168.30 (C═O Phth), 136.87 (Cq arom. Aba), 135.44 (CH arom. Phth), 132.04 (Cq arom. Phth), 130.43 (CH arom. Aba), 128.95 (CH arom. Aba), 127.36 (CH arom. Aba), 123.95 (CH arom. Phth), 52.35 (CH_{α} Aba), 51.03 (CH_{2ε} Aba), 48.37 (CH_{2γ} GABA), 33.57 (CH_{2β} Aba), 31.41 (CH_{2α} GABA), 23.61 (CH_{2β} GABA).

Synthesis of Fmoc-Aba-GABA-OH

To a 100 mL round bottom flask, Phth-Aba-GABA-OH (0.674 g, 1.72 mmol), hydrazine monohydrate (543 μL, 11.2 mmol) and EtOH (20 mL) are added. The reaction mixture was refluxed at 90° C. for 2 h then evaporated and dried under vacuum for 2 h. The residue was dissolved in 10 mL water then pH was adjusted from 8 to 4 by acetic acid addition. The resulting suspension was stirred at rt for 30 min then filtered and concentrated to give the Phth-deprotected intermediate used without further purification.

The residue was dissolved in acetone (10 mL) and water (10 mL). Then a solution of Fmoc-OSu (0.608 g, 1.80 mmol) and Na₂CO₃ (0.210 g, 1.98 mmol) in water (10 mL) and acetone (10 mL) was added. The reaction mixture was stirred at rt overnight under Ar atmosphere. Acetone was then evaporated and the resulting aqueous solution was acidified to pH 2 using a 6M HCl solution. The suspension was extracted with ethyl acetate (4 times) and the resulting organic phases were combined, washed once with brine, dried with MgSO₄, filtered and concentrated. The crude was finally purified by column chromatography using DCM/MeOH 98/2 with 1% acetic acid as eluent yielding the desired compound (0.564 g, 68%).

Formula: C₂₉H₂₈N₂O₅; MW: 484.20 g/mol; HPLC: t_{R}=2.82 min; LC-MS (ES+): [M+H]+=485.19 Da; ¹H NMR (251 MHz, CDCl₃) δ (ppm) 7.68 (m, 4H, H arom.), 7.36 (m, 4H, H arom.), 7.24-7.01 (m, 4H, H arom.), 6.36 (d, J=6.6 Hz, 1H), 5.41-5.02 (m, 2H, H_{α} and H_{ε} Aba), 4.39 (d, J=7.2 Hz, 2H, CH2 Fmoc), 4.24 (t, J=7.2 Hz, 1H, CH Fmoc), 3.89 (d, J=16.7 Hz, 1H, H_{ε}′), 3.65-3.37 (m, 3H, H_{β} Aba and 2H_{γ} GABA), 2.98 (dd, J=16.9, 13.1 Hz, 1H, H_{β}′), 2.41-2.11 (m, 2H, 2H_{α} GABA), 1.84 (m, 2H, 2H_{β} GABA), 13C NMR (63 MHz, CDCl3) δ (ppm) 177.2 (C═O—COOH), 171.8 (C═O amide), 155.9 (C═O Fmoc) 144.1 (Cq arom. Fmoc), 141.4 (Cq arom. Fmoc), 135.6 (Cq arom. Aba), 133.2 (Cq arom. Aba), 131.1 (CH arom. Fmoc) 128.7 (CH arom. Aba), 128.3 (CH arom. Fmoc), 127.8 (CH arom. Aba), 127.2 (CH arom. Aba), 126.5 (CH arom. Aba), 125.4 (CH arom. Fmoc), 120.2 (CH arom. Fmoc), 67.3 (CH₂ Fmoc), 52.4 (CH_{2ε} Aba), 50.0 (CH_{α} Aba), 47.3 (CH Fmoc and CH_{2γ} GABA), 37.0 (CH_{2β} Aba), 30.9 (CH_{2α} GABA), 23.2 (CH_{2β} GABA).

### 1.2.13. Synthesis of Fmoc-Apa-OH, Fmoc-Bpa-OH, Fmoc-D-Bpa-OH, Fmoc-THIQ-OH

N-protected amino acids N-Fmoc-Apa-OH, N-Fmoc-Bpa-OH, N-Fmoc-D-Bpa-OH and N-Fmoc-THIQ-OH were prepared as described by Schneider and al. [46]
1.3. Receptor cDNA Constructs, Cell Expression and Membrane Preparations
Human embrionic kidney 293 (HEK293) cells expressing Glo-sensor 20F were a gift from M. Hanson (GIGA, Liège, Belgium). Human MOPr, DOPr, KOPr, NOPr, NPFF1R, NPFF2R and GPR54 cDNAs were subcloned into the pCDNA3.1 expression vector (Invitrogen, Cergy Pontoise, France) and transfected into Chinese Hamster ovary (CHO) cells or HEK293-Glo-20F cells before selection for stable expression, as reported [13]. CHO cells expressing human GPR10 and GPR103 were a gift from M. Parmentier (IRIBHM, Brussels, Belgium). All cell membranes were prepared as described [13] and stored at −80° C. as aliquots (1 mg prot/mL) until use.
1.4. Radioligand binding assays
Binding assay conditions were essentially performed as described [13]. Briefly, membranes from CHO cells stably expressing human GPR10, GPR54, GPR103, NPFF1R or NPFF2R were incubated with 0.6 nM [³H]-PrRP-20, 0.05 nM [¹²⁵I]-Kp-10, 0.03 nM [¹²⁵I]-43RFa, or 0.015 nM [¹²⁵I]-1-DMe-NPFF (for NPFF1R and NPFF2R), respectively. Membranes from HEK293 cells stably expressing human MOP, DOP and KOP receptors were incubated with 1 nM [³H]-diprenorphine. Membranes from HEK293 cells transiently expressing human NOP receptors were incubated with 0.2 nM [³H]-nociceptin. Competition binding experiments were performed at 25° C., under equilibrium conditions (60 min, 0.25 mL final volume), in the presence of increasing concentrations of unlabeled peptides or test compounds. Membrane-bound radioactivity was separated from free radioligand by rapid filtration through a 96-well GF/B unifilter apparatus (Perkin Elmer Life and Analytical Sciences, Courtaboeuf, France) and quantified using a TopCount scintillation counter (Perkin Elmer).

### 1.5. [³⁵S]-GTPγS Binding Assay

Stimulation by endogenous RF-amide peptides or test compounds for [³⁵S]-GTPγS binding to membranes from CHO cells expressing human NPFF1R or NPFF2R, were examined as reported [48].
1.6. Glo-Sensor cAMP Assay
cAMP accumulation assay was essentially done as described [45] with the following modifications: HEK293 cells stably expressing cAMP Glosensor-20F were used, with or without additional stable expression of each individual opioid receptor or NPFF1R. The cAMP responses were measured in presence of D-luciferin (1 mM). All peptides and test compounds were incubated in the presence of IBMX for 15 min before inducing cAMP production by forskolin. IBMX and forskolin concentrations were optimized for each receptor cell line: NPFF1R responses were recorded in presence of 0.1 mM IBMX and 0.4 μM forskolin, NPFF2R responses with 0.5 mM IBMX and 0.3 μM forskolin, MOPr responses with 0.5 mM IBMX and 0.125 μM forskolin, DOPr responses with 0.1 mM IBMX and 0.125 μM forskolin; KOPr and NOPr with 0.5 mM IBMX and 1.5 μM forskolin. The antagonist activity of the derivatives on NPFF1R and NPFF2R was evaluated at three different concentrations (0.5, 5 and 50 μM) in the presence of 50 nM RFRP3 and 200 nM NPFF, respectively.

### 1.7. Calcium Mobilization Assay

CHO cells expressing human NPFF2R were loaded with 2.5 μM Fluo-4 AM in the presence of 2.5 mM probenecid, as described previously [13]. Agonist-evoked increases in intracellular calcium were recorded over time (5 sec intervals over 220 sec) at 37° C. through fluorescence emission at 520 nm (excitation at 485 nm). Peak response amplitudes were normalized to basal and maximal (cells permeabilized with 20 μM digitonin) fluorescence levels.
1.8. β-Arrestin-2 recruitment assay
The β-arrestin-2 recruitment assay was performed as described [34; 45] with minor modifications. Briefly, two days prior the experiment, HEK293 cells stably expressing eYFP-tagged β-arrestin-2 were transfected with the plasmid encoding Rluc8-MOP receptor. β-Arrestin-2 recruitment was measured at 37° C. in presence of 5 μM Coelenterazine H, 5 min after agonist addition. A “BRET ratio” corresponding to the signal in the “acceptor channel” (band-pass filter 510-560 nm) divided by the signal in the “donor channel” (band-pass filter 435-485 nm) was calculated. Drug-induced BRET was determined (BRET1 ratio of drug-activated cells minus BRET1 ratio of buffer-treated cells) and normalized to the maximum of DAMGO-induced BRET, defined as 100%.

### 1.9. In Vivo Experiments

All experiments were carried out in accordance with the European guidelines for the care of laboratory animals (European Communities Council Directive 2010/63/EU) and were approved by the local ethical committee and authorized by the French Ministry for Research and the Committee of Animal Care of the Austrian Federal Ministry of Science and Research. All efforts were made to minimize animal discomfort and to reduce the number of animals used.

### 1.10. Animals

Animal experiments were performed on adult male C57BL/6N male mice (25-30 g weight; Janvier labs, France). Animals were housed in groups of three to five per cage and kept under a 12 h/12 h light/dark cycle at 21±1° C. with ad libitum access to food and water. Experiments were performed during the light-on phase of the cycle. Mice were habituated to the testing room and equipment before starting behavioral experiments. Control and treated group assignment as well as pain response measurements were performed in a blinded manner. Every animal was used only once.
1.11. Drug administration
All drugs were dissolved in physiological saline (0.9%) and administered subcutaneously (sc.) at 10 mL/kg (volume/body weight).
1.12. Assessment of thermal nociception
The nociceptive sensitivity to thermal stimulation was determined in mice using the warm-water tail immersion tests as previously described [12; 49]. In the tail immersion test, C57BN/6N mice were restrained in a grid pocket and their tail was immersed in a thermostated water bath. The latency (in sec) for tail withdrawal from hot water (47.5±0.5° C.) was taken as a measure of the nociceptive response. In the absence of any nociceptive reaction, a cut-off value of 25 sec was set to avoid tissue damage.
Experiments for chronic drug effects were designed according to a protocol enabling the evaluation of time-course of opioid-induced hyperalgesia and development of analgesic tolerance using the tail immersion test. C57BL/6N mice were sc. treated daily with 1.8 μmol/kg/d KGOP01, 1.2 μmol/kg/d KGFF03, 7.4 μmol/kg/d KGFF09 or saline (controls) for 8 days. For analgesic tolerance evaluation, nociceptive latencies were measured on day 1 and 8 according to the acute effect protocol. For the assessment of hyperalgesia, basal nociceptive latencies were measured every day, 30 min before drug or saline injection. Responses are expressed as latency times (in sec) of tail withdrawal from the hot water.

### 1.13. CFA-Induced Inflammatory Pain Model

Tail inflammation in C57BL/6N mice was induced by injecting subcutaneously 20 μl of a Complete Freund's Adjuvant (CFA) solution or saline (control mice) 3 cm from the tip of the tail [41]. Twenty-four hours after CFA injection (day 1), inflammation was confirmed by measuring thermal and mechanical hyperalgesia. Mice were then treated sc. daily for 7 days (from day 1 to day 7) with the test compounds or saline (control). Nociceptive threshold to heat stimulation was measured by tail immersion test (47.5±0.5° C.) and tail pressure test [12] each hour for 5 h after the first drug administration in order to determine the peak of the anti-hyperalgesic response. During the following days, basal nociceptive thresholds were evaluated before injection and 2 h after drug injection. Antinociceptive response was calculated as percent of Maximum Possible Effect (% MPE) according to the formula=[(test latency−CFA/saline mice latency)/(cut-off time−CFA/saline mice latency)]×100. In order to limit behavioral sensitization, thermal nociception was evaluated on days 1-2-4-6 and mechanical nociception on days 1-3-5-7.

### 1.14. Naltrexone-Precipitated Withdrawal Syndrome

Opioid physical dependence was induced in C57BL/6N mice by sc. administration of test compounds twice daily, 1.8 μmol/kg KGOP01, 1.2 μmol/kg KGFF03, 7.4 μmol/kg KGFF09 or saline (control) over a 7-days period. On day 7, two hours after the last drug injection, the withdrawal syndrome was precipitated by administration of naltrexone (5 mg/kg, sc.) and evaluated over 30 min. Jumping, paw tremors and wet dog shakes were recorded as number of events occurring during the total test time. Diarrhea was checked for 30 min with one point given for any signs of it during each 5 min period (maximum score: 6). Body weight was measured immediately before and after each 30 min test session, and percentage of body weight lost during the test was calculated. For each mouse, a global opiate withdrawal score was also calculated by summing the values obtained for each sign. For this purpose one point was assigned to every 3 jumps and 5 paw tremors, respectively, whereas all other signs were given the absolute values recorded during the test [39].

### 1.15. Respiratory Depression Measurement Using Whole Body Plethysmography

Ventilatory parameters were recorded in conscious C57BL/6N mice by whole body barometric plethysmography (Emka Technologies, Paris, France). Mice were acclimatized with the plethysmograph chamber for 30 min until a stable baseline was obtained. Then, the animal was gently removed from the chamber for sc. injection of the tested drug at TO and replaced in the chamber for the remaining measurements. Respiratory frequency (f) was recorded for 100 min and used as the index of respiratory depression [31].

### 1.16. Statistics

For in vitro binding and functional experiments, two to four independent experiments were performed in duplicates and data were analyzed using Prism (GraphPad Software, San Diego, Calif., USA). In vivo data are expressed as mean values±SEM for 6 to 12 mice per group. Antinociception was quantified as the area under the curve (AUC) calculated by the trapezoidal method [8]. Data were analyzed using one-way or two-way analysis of variance (ANOVA). Post-hoc analyses were performed with Bonferroni tests. The level of significance was set at p<0.05. All statistical analyses were carried out using the StatView or GraphPad Prism softwares.

2. Results

### 2.1. Design Strategy and Biological Screening of MOPr/NPFFR Peptidomimetic Ligands

In this study, the inventors designed a series of bifunctional ligands that were based on a combination of the recently described MOPr peptidomimetic KGOP01 [20], found to display potent analgesic activity following systemic administration, and standard but also more evolved NPFF pharmacophores (FIGS. 1 A and B). The sequences of synthetized peptidomimetics are shown in Table 1.

TABLE 1

| compound | Sequence | | | | | |
|---|---|---|---|---|---|---|
| position | X1 | X2 | X3 | X4 | X5 | X6-T |
| KGFF01 | H-Dmt | D-Arg | Aba | Gly | Arg | Phe-NH₂ |
| KGFF02 | H-Dmt | D-Arg | Aba | Gly | Arg | Phe-OH |
| KGFF03 | H-Dmt | D-Arg | Aba | b-Ala | Arg | Phe-NH₂ |
| KGFF04 | H-Dmt | D-Arg | Aba | Gly | Orn | Phe-NH₂ |
| KGFF05 | H-Dmt | D-Arg | — | Phe | Orn | Phe-NH₂ |
| KGFF06 | H-Dmt | L-Arg | — | — | — | Phe-NH₂ |
| KGFF07 | H-Dmt | D-Arg | — | — | — | Phe-NH₂ |
| KGFF08 | H-Dmt | D-Arg | Aba | b-Ala | Apa | Phe-NH₂ |
| KGFF09 | H-Dmt | D-Arg | Aba | b-Ala | Bpa | Phe-NH₂ |
| KGFF10 | H-Dmt | — | — | — | Apa | Phe-NH₂ |
| KGFF11 | H-Dmt | — | — | — | Bpa | Phe-NH₂ |
| KGFF12 | H-Dmt | D-Arg | Aba-NH | — | — | — |
| KGFF13 | H-Dmt | D-Arg | — | — | — | Phe-NH—CH₃ |
| KGFF14 | H-Dmt | D-Arg | Aba | b-Ala | Lys(Bim) | Phe-NH₂ |
| KGFF15 | H-Dmt | D-Arg | Aba | b-Ala | Lys(Box) | Phe-NH₂ |
| KGFF16 | H-Dmt | D-Arg | Aba | b-Ala | Lys(Bth) | Phe-NH₂ |
| KGOP-01 | H-Dmt | D-Arg | Aba | b-Ala-NH₂ | | |

As a standard NPFF pharmacophore, the C-terminal RF-NH2 dipeptide segment of NPFF served as a minimal recognition motif (structure 2, FIG. 1A). Many of the reported NPFF analogues contain the C-terminal Arg-Phe-amide, derived with an apolar moiety linked at its N-terminus [16; 18; 23; 33; 48]. Combination of both pharmacophores (Table 1) afforded KGFF01 and KGFF03 that showed nanomolar affinity for MOPr and NPFF1/2R (FIG. 1C and Table 2a, which summarize affinity constant (Ki) values of KGFF compounds for MOPr, NPFF1R, and NPFF2R). The amino-benzazepinone (Aba) scaffold could act as the apolar group that is often present in previously reported NPFF ligands. The insertion of a Gly (KGFF01) or a β-Ala (KGFF03) in position 4, i.e. between the RF-NH2 dipeptide and the benzazepinone ring, provided good affinity for NPFF1/2R with preserved MOPr affinity, while eliminating a potential steric hindrance imposed by the NPFF pharmacophore onto the opioid part. The corresponding C-terminal carboxylic acid (KGFF02) confirmed the importance of the C-terminal amide in this type of hybrid with a strong decrease of affinity for both NPFF1/2R. Likewise, the substitution of the Arg residue by Orn (KGFF04, KGFF05) was not well tolerated, resulting in reduced binding affinities to both NPFF1/2R subtypes (FIG. 1C and Table 2a, which summarize affinity constant (Ki) values of KGFF compounds for MOPr, NPFF1R, and NPFF2R).
Further truncation of KGFF01 gave way to tripeptides KGFF06 and KGFF07 with complete overlap of the opioid and NPFF pharmacophores, as the 2,6-dimethyl tyrosine (Dmt) side chain could efficiently serve as the apolar group. In this case, the opioid segment is cut down to three amino acids instead of four, with well-conserved MOPr binding affinity for KGFF07. In addition to the detrimental consequence on MOPr affinity, the stereochemistry of Arg (L-Arg for KGFF06 and D-Arg for KGFF07) seems to drive a selectivity switch between NPFF1R and NPFF2R (FIG. 1C and Table 2a, which summarize affinity constant (Ki) values of KGFF compounds for MOPr, NPFF1R, and NPFF2R).
Following initial in vitro biological evaluation of the described hybrid peptidomimetics, the two peptide analogues, KGFF03 and KGFF07, with the most promising binding data on MOPr and NPFFR were used to design a second set of bifunctional peptides (Table 1). Within this second set, the Arg residues were replaced by both reported Orn-derivatives (FIG. 1B, 3, Apa [2-amino-5-(piperidin-1-yl)pentanoic acid] and 4, Bpa [2-amino-5-(4-benzylpiperidin-1-yl)pentanoic acid]) [46], as well as novel Arg mimetics (5 to 7, FIG. 1B; see Methods for synthetic details). Structural modifications on KGFF07 were first explored. Substitution of L- or D-Arg2 by 3 and 4 using standard Fmoc-SPPS gave rise to tripeptide analogues KGFF10 and KGFF11, with a consequential dramatic loss of affinity for MOPr and NPFFR. In an attempt to lower the molecular flexibility of this tripeptide, the C-terminal Phe was constrained through incorporation of the amino-benzazepinone (Aba) core (KGFF12), resulting in a significant drop in binding affinity for NPFF1R and to a lesser extent for NPFF2R. In order to verify if the decrease in affinity was due to the incorporation of the methylene bridge providing the cycle or from the conversion of a primary to a secondary amide, the ‘open ring’ analogue KGFF13 was also synthetized. This compound displayed a partially restored affinity for the NPFF1R and NPFF2R (FIG. 1C and Table 2a, which summarize affinity constant (Ki) values of KGFF compounds for MOPr, NPFF1R, and NPFF2R).
It has been recently shown that the guanidine moiety of Arg can be advantageously replaced by tertiary amines in the sequence RF-NH2, leading to new peptidomimetic ligands of NPFFRs [5]. Based on the promising biological data of KGFF01 and KGFF03, and in light of the previous work, the Arg5 residue was replaced by piperidine- and benzylpiperidine-bearing residues 3 and 4, respectively (FIG. 1B), to give the hybrid products KGFF08 and KGFF09 (Table 1). The Arg mimetics (5 to 7) were incorporated at the same position in this sequence leading to KGFF14, KGFF15 and KGFF16 (Table 1). Substitution of Arg5 in KGFF03 with these Arg mimetics led in general to good binding affinity for both MOPr and NPFF1/2Rs (MOPr: Ki<10 nM; NPFF1R: Ki<150 nM; NPFF2R: Ki<15 nM) with a globally better affinity for the NPFF2R over NPFF1R subtype, especially after incorporation of Apa, Bpa and Lys(Box) residues. Based on these data, hybrid peptidomimetics KGFF01/-03/-07/-08/-09/-14/-15/-16 were selected for further characterization of their activity on MOPr and NPFF1/2R in functional assays.
The Table 2b summarizes 1050 values of DP compounds for NPFF1R and NPFF2R. These compounds displayed nanomolar affinity for NPFF1/2R,

TABLE 2a
Binding affinity constant (Kᵢ) values of KGFF
compounds for human MOPr, NPFF1R and NPFF2R.
Ki ± SEM (nM)

| compound | MOPr | NPFF1R | NPFF2R |
|---|---|---|---|
| DAMGO | 14.6 ± 3.8 | nd | nd |
| RFRP3 | nd | 0.053 ± 0.003 | nd |
| NPFF | nd | nd | 0.28 ± 0.16 |
| KGOP01 | 0.12 ± 0.02 | 4,600 ± 1,300 | 4,500 ± 1,800 |
| KGFF01 | 0.59 ± 0.21 | 57 ± 10 | 1.2 ± 0.5 |
| KGFF02 | 0.59 ± 0.15 | 2,540 ± 460 | 251 ± 25 |
| KGFF03 | 0.24 ± 0.03 | 2.7 ± 0.3 | 0.077 ± 0.01 |
| KGFF04 | 0.67 ± 0.29 | 780 ± 90 | 45 ± 8 |
| KGFF05 | 3.2 ± 1.1 | 3,100 ± 1,100 | 360 ± 50 |
| KGFF06 | 46 ± 22 | 147 ± 20 | 15.9 ± 1.8 |
| KGFF07 | 0.29 ± 0.13 | 11.8 ± 3.6 | 84 ± 26 |
| KGFF08 | 0.94 ± 0.19 | 136 ± 26 | 3.9 ± 0.1 |
| KGFF09 | 2.43 ± 0.18 | 83 ± 21 | 3.2 ± 0.7 |
| KGFF10 | 68 ± 48 | >10,000 | 2,100 ± 600 |
| KGFF11 | >10,000 | 2,720 ± 760 | 406 ± 95 |
| KGFF12 | 0.17 ± 0.05 | 2,490 ± 1,170 | 375 ± 108 |
| KGFF13 | 0.11 ± 0.05 | 147 ± 16 | 206 ± 47 |
| KGFF14 | 1.6 ± 0.3 | 4.54 ± 0.01 | 4.15 ± 1.35 |
| KGFF15 | 1.8 ± 0.9 | 88 ± 8 | 9.6 ± 0.6 |
| KGFF16 | 2.6 ± 1.3 | 80 ± 13 | 10.4 ± 2.3 |

Data are mean ± SEM of at least 2 independent experiments performed in duplicate. Kᵢ values were determined from competition binding curves using [³H]-diprenorphine for MOPr, and [¹²⁵I]-1-DMe-NPFF for NPFF1R and NPFF2R.

TABLE 2b
IC₅₀ values of DP compounds on NPFF1R and NPFF2R.
IC50 (nM)

| compound | NPFF1R | NPFF2R |
|---|---|---|
| DP0001 | ≈500 | <50 |
| DP0002 | 50-500 | <50 |
| DP0003 | 50-500 | <50 |
| DP0004 | 50-500 | <50 |
| DP0005 | ≈50 | <50 |
| DP0007 | nd | nd |
| DP0008 | nd | nd |
| DP0009 | nd | nd |
| DP0012 | <40 | <40 |
| DP0013 | <40 | ≈40 |
| DP0014 | <40 | ≈40 |
| DP0015 | <40 | <40 |
| DP0016 | nd | nd |
| DP0017 | nd | nd |
| DP0018 | 40-400 | <40 |
| DP0019 | 40-400 | 40-400 |
| DP0020 | 40-400 | 40-400 |
| DP0021 | 40-400 | <40 |
| DP0022 | ≈40 | <40 |
| DP0023 | nd | nd |
| DP0024 | nd | nd |
| DP0025 | nd | nd |
| DP0026 | nd | nd |
| DP0027 | nd | nd |
| DP0028 | nd | nd |
| DP0029 | nd | nd |
| DP0030 | nd | nd |
| DP0031 | nd | nd |
| DP0032 | nd | nd |
| DP0033 | nd | nd |
| DP0034 | nd | nd |
| DP0035 | nd | nd |

IC50 values were estimated from a single competition binding experiment performed in duplicates in the presence of [¹²⁵I]-1-DMe-NPFF for NPFF1R and NPFF2R and three concentrations of each compounds: 0.05, 0.5 and 5 μM for DP0001 to DP0005 and 0.04, 0.4 and 4 μM for DP0012 to DP0015 and DP0018 to DP0022.
nd: not determined

### 2.2. Identification of an MOPr/NPFFR Agonist (KGFF03) and a Mixed MOPr Agonist/NPFFR Antagonist (KGFF09)

The capacity of the selected compounds to inhibit forskolin-induced cAMP production from MOPr overexpressing HEK cells was first evaluated. All ligands displayed full agonist activity at the MOPr (EC₅₀ values ranging from 1.5 to 18.2 nM, as compared to DAMGO, EC₅₀=80 nM, FIG. 2A and Table 3a, which summarize agonist activity constants (Ec₅₀ and Eₘₐₓ) values of KGFF compounds for MOPr, NPFF1R, and NPFF2R). The functional activity of the KGFF compounds on both NPFF1R and NPFF2R in the [³⁵S]-GTPγS binding assay was then evaluated (FIGS. 2 B and C, and Table 3a, which summarize agonist activity constants (Ec₅₀ and Eₘₐₓ) values of KGFF compounds for MOPr, NPFF1R, and NPFF2R). KGFF03 was the most potent agonist for both NPFF1/2R (NPFF1R: EC₅₀=84.8 nM; NPFF2R: EC₅₀=11 nM), whereas KGFF09 was the only ligand to show no or weak agonist activity at both NPFF1R and NPFF2R. The absence of agonist activity of KGFF09 on NPFFRs was further confirmed by using integrated cellular assays (FIGS. 6 A and C, which shows in vitro characterization of KGFF03 and KGFF09 on NPFFRs). Finally, it was shown that KGFF09 displayed potent antagonist activity at NPFF1/2Rs (FIGS. 2 E and F, FIGS. 6 B and D, which shows in vitro characterization of KGFF03 and KGFF09 on NPFFRs) with pA₂ values of 7.25±0.30 and 7.77±0.21, respectively, calculated from [³⁵S]-GTPγS binding experiments.

Overall, these data allowed us to identify at least one molecule, KGFF03, that displays potent MOPr and NPFFR agonist activities, and at least a second ligand, KGFF09, that shows mixed MOPr agonist and NPFF1/2R antagonist activities. These two ligands underwent further in vitro and in vivo characterization, and their profiles were compared to the parent opioid ligand KGOP01.
Table 3b summarize agonist and antagonist activity constant values of DP compounds on MOP (agonist) and NPFFR1/2 (Agonist and antagonist). These compounds display mixed MOPr agonist and NPFF1/2R antagonist (and potentially partial agonist for DP0001, 0002 and 0003) activities.

TABLE 3a
Agonist activity constant (EC₅₀ and Eₘₐₓ) values of KGFF compounds for human MOPr, NPFF1R and NPFF2R.

| | MOPr | | MOPr | | NPFF1R | | NPFF2R | |
|---|---|---|---|---|---|---|---|---|
| | (cAMP) | | (β-arrestin-2) | | (GTPγS) | | (GTPyS) | |
| | EC₅₀ | Eₘₐₓ | EC₅₀ | Eₘₐₓ | EC₅₀ | Eₘₐₓ | EC₅₀ | Eₘₐₓ |
| compound | (nM) | (%) | (nM) | (%) | (nM) | (%) | (nM) | (%) |
| DAMGO | 80.4 ± 23.2 | 100 ± 4 | 240 ± 65 | 100 ± 2 | nd | | nd | nd |
| RFRP3 | nd | | | | 10.1 ± 2.8 | 100 ± 10 | nd | nd |
| NPFF | nd | | | | nd | nd | 18.2 ± 6.4 | 100 ± 4 |
| KGOP01 | 0.204 ± 0.05 | 89 ± 2 | 1.6 ± 0.1 | 103 ± 9 | >10,000 | nd | >10,000 | nd |
| KGFF01 | 2.4 ± 1.0 | 101 ± 7 | | | 155 ± 25 | 44 ± 8 | 128 ± 23 | 95 ± 6 |
| KGFF02 | 1.5 ± 0.7 | 89 ± 2 | | | nd | nd | nd | nd |
| KGFF03 | 12.0 ± 2 | 92 ± 1 | 33.9 ± 8.8 | 41 ± 11 | 84.8 ± 22.2 | 88 ± 7 | 11 ± 2 | 111 ± 17 |
| KGFF04 | 9.5 ± 1.5 | 70 ± 4 | | | >10,000 | 25 ± 4 | 963 ± 168 | 67 ± 3 |
| KGFF05 | 59.3 ± 24.5 | 94 ± 2 | | | nd | nd | nd | nd |
| KGFF06 | 2,300 ± 300 | nd | | | nd | nd | nd | nd |
| KGFF07 | 1.7 ± 0.4 | 102 ± 2 | | | 87 ± 7 | 84 ± 8 | 1,930 ± 66 | 87 ± 2 |
| KGFF08 | 9.9 ± 2.0 | 102 ± 3 | | | >10,000 | 23 ± 1 | 574 ± 129 | 97 ± 2 |
| KGFF09 | 18.2 ± 6.1 | 85 ± 2 | 56.4 ± 26.5 | 42 ± 2 | 176 ± 66 | 10 ± 2 | 157 ± 49 | 38 ± 14 |
| KGFF10 | 598 ± 35 | 91 ± 10 | | | nd | nd | nd | nd |
| KGFF11 | >10,000 | nd | | | nd | nd | nd | nd |
| KGFF12 | 2.7 ± 1.5 | 103 ± 4 | | | >10,000 | 13 ± 3 | >10,000 | 32 ± 17 |
| KGFF13 | 1.0 ± 0.4 | 97 ± 2 | | | >10,000 | 39 ± 2 | >10,000 | 68 ± 8 |
| KGFF14 | 5.9 ± 2.5 | 99 ± 4 | | | 76 ± 26 | 74 ± 1 | 89 ± 7 | 73 ± 18 |
| KGFF15 | 4.1 ± 1.7 | 92 ± 13 | | | 1,020 ± 610 | 51 ± 5 | 930 ± 210 | 105 ± 15 |
| KGFF16 | 6.2 ± 2.0 | 93 ± 6 | | | 273 ± 115 | 51 ± 7 | 526 ± 12 | 88 ± 12 |

Efficacy (Eₘₐₓ) is expressed as the percentage relative to the reference compound (DAMGO, RFRP3 and NPFF for MOPr, NPFF1R and NPFF2R, respectively). Values are mean ± SEM of at least 2 independent experiments performed in duplicate.
nd, not determined.

TABLE 3B
Agonist activity (EC50 and Emax) of DP compounds for human MOPr and
agonist and/or antagonist activity (IC50) for NPFF1R and NPFF2R.

| | | | | | NPFF1R | | NPFF2R | |
|---|---|---|---|---|---|---|---|---|
| | MOPr | | MOPr | | (cAMP) | | (cAMP) | |
| | (cAMP) | | (β-arrestin-2) | | | Antagonist | | Antagonist |
| | EC₅₀ | Eₘₐₓ | EC₅₀ | Eₘₐₓ | Agonist | mode | Agonist | mode |
| compound | (nM) | (%) | (nM) | (%) | mode | IC50 (μM) | mode | IC50 (μM) |
| DAMGO | 80.4 ± 23.2 | 100 ± 4 | 240 ± 65 | 100 ± 2 | nd | nd | nd | nd |
| KGFF09 | 18.2 ± 6.1 | 85 ± 2 | 56.4 ± 26.5 | 42 ± 2 | — | ≈5 | — | ≈5 |
| DP0001 | 16.9 ± 8.3 | 124 | 101 | 52 | — | ≈5 | Partial | ≈50 |
| DP0002 | 8.9 ± 2.6 | 110 | 45 | 49 | — | ≈5 | Partial | ≈50 |
| DP0003 | 20.1 ± 10 | 70 | 37 | 38 | — | ≈5 | Partial | ≈50 |
| DP0004 | 4.2 ± 0.7 | 88 ± 4 | 126 | 64 | — | <5 | — | ≈5 |
| DP0005 | 7.9 ± 0.5 | 81 ± 0.6 | 98 | 42 | — | <5 | — | ≈5 |
| DP0007 | 40 ± 20 | 64 ± 6 | 214 | 12 | — | <5 | — | ≈5 |
| DP0008 | 10.6 ± 1.3 | 53 ± 0.7 | — | — | — | <5 | — | ≈5 |
| DP0009 | 800 | 35 | nd | nd | — | ≈5 | — | ≈5 |
| DP0012 | 15.1 ± 0.4 | 90 ± 2 | 650 | 22 | — | >5 | — | <5 |
| DP0013 | 0.53 ± 0.004 | 115 ± 4 | 20.5 | 101 | — | >5 | — | ≈5 |
| DP0014 | 5.3 | 76 | — | — | — | >5 | — | ≈5 |
| DP0015 | 5.9 ± 0.3 | 109 ± 0.4 | 80 | 59 | — | ≈5 | — | >5 |
| DP0016 | 19 | 95 | 399 | 31 | nd | >5 | nd | >5 |
| DP0017 | 14.1 | 70 | — | — | nd | >5 | nd | ≈5 |
| DP0018 | 5.5 | 108 | 95.8 | 31 | — | ≈5 | — | >5 |
| DP0019 | 4 ± 0.4 | 101 ± 1 | 162 | 53 | — | >5 | — | >5 |
| DP0020 | 6.3 | 101 | 149 | 53 | — | >5 | — | >5 |
| DP0021 | 2.8 ± 0.4 | 102 ± 3 | 109 ± 57 | 22 | — | ≈5 | — | <5 |
| DP0022 | 5 | 90 | 617 ± 205 | 30 ± 0.7 | — | ≈5 | — | ≈5 |
| DP0023 | 9.1 | 61 | — | — | — | ≈5 | — | <5 |
| DP0024 | 9.9 | 97 | ≈50 | ≈95 | nd | >5 | nd | ≈5 |
| DP0025 | 7.1 | 110 | ≈50 | ≈114 | nd | — | nd | ≈50 |
| DP0026 | 2.4 | 98 | ≈50 | ≈50 | nd | >5 | nd | ≈50 |
| DP0027 | 13.6 | 95 | ≈180 | ≈68 | nd | >5 | nd | ≈5 |
| DP0028 | 40.4 | 75 | — | — | nd | >5 | nd | ≈5 |
| DP0029 | 25.7 | 72 | — | — | nd | >5 | nd | >50 |
| DP0030 | 27.7 | 111 | ≈150 | ≈85 | nd | >5 | nd | >50 |
| DP0031 | 3600 | 46 | >500 | ≈40 | nd | ≈50 | nd | ≈5 |
| DP0032 | 12.6 | 63 | — | — | nd | <5 | nd | <5 |
| DP0033 | 24.9 | 41 | — | — | nd | ≈5 | nd | <5 |
| DP0034 | 3.9 | 92 | 5-50 | ≈18 | nd | >5 | nd | <5 |
| DP0035 | 48.4 | 15 | — | — | nd | nd | nd | nd |

For MOR: Efficacy (Eₘₐₓ) is expressed as the percentage relative to the reference compound DAMGO. Agonist activity at NPFF1R and NPFF2R of each compound was evaluated et 2 concentrations 0.5 and 5 μM. Antagonist activity at NPFF1R and NPFF2R of each compound was evaluated in the presence of 50 nM RFRP3 and 200 nM NPFF (respectively) and three concentrations (0.5, 5 and 50 μM) of test compound.
nd: not determined.

### 2.3. KGFF03 and KGFF09 are G Protein-Biased MOPr Agonists

While opioid-induced analgesia is attributed to MOPr signaling through the G protein Gᵢ, β-arrestin-2 recruitment upon MOPr activation is suggested to be responsible for many acute side effects including respiratory depression and constipation [10; 31; 43]. To examine MOPr biased agonism of KGOP01, KGFF03 and KGFF09 towards activation of G protein-over β-arrestin-2-mediated signaling, their functional activity, i.e. potency and efficacy, was compared across two cell-based assays that measure G protein coupling (the cAMP accumulation assay) and β-arrestin-2 translocation (the BRET1 β-arrestin-2 recruitment assay) at the human MOPr (FIGS. 2 A and D, and Table 3a), which summarize agonist activity constants (EC₅₀ and Eₘₐₓ) values of KGFF compounds for MOPr, NPFF1R, and NPFF2R). Both KGFF03 and KGFF09 elicited partial agonism for β-arrestin-2 recruitment, while being highly potent and fully efficacious in promoting MOPr-induced G protein activation. In contrast, KGOP01 robustly stimulated interaction between the MOPr with G protein and β-arrestin-2 with a full response and potency, as compared to DAMGO. These data indicate that the two newly designed MOPr/NPFFR hybrid ligands, for example KGFF03 and KGFF09, activate MOPr in a manner that is preferentially biased toward G protein signaling. The compounds DP0008, DP0014, DP0017, DP0023, DP0028, DP0029, DP0032, DP0033 display significant MOPr-induced G protein activation with non-detectable β-arrestin-2 recruitment (Table 3b).

### 2.4. Selectivity of KGFF03 and KGFF09 at Opioid and RF-Amide Receptors

The binding affinity and functional activity of KGFF03, KGFF09 and KGOP01 at other opioid receptor types were further investigated (Table 4, which summarize binding affinity constant (K) values and agonist activity constant (EC₅₀ and Eₘₐₓ) values of KGFF compounds for human DOPr, KOPr, and NOPr and FIG. 7, which shows in vitro characterization of

KGOP01, KGFF03 and KGFF09 on opioid receptors). KGFF03 showed good affinity at the DOPr, and lower affinity for KOPr and NOPr. It displayed potent agonist activity at DOPr (EC₅₀=0.34 nM) and lower agonist activity at KOPr (EC₅₀=95 nM), as well as very weak antagonist activity at NOPr (pA₂=5.3±0.34). KGFF09 showed lower affinity at the DOPr and relatively higher affinity for the KOPr, in comparison with KGOP01 and KGFF03. Its affinity for the NOPr was similar to KGFF03. Alike KGOP01 and KGFF03, KGFF09 displayed potent agonist activity at DOPr (EC₅₀=0.78 nM). Moreover, KGFF09 displayed potent antagonist activity at the KOPr (pA₂=8.16±0.13), but low antagonist activity at the NOPr (pA₂=5.94±0.12).

Because NPFFRs belong to the family of RF-amide receptors, which include GPR10, GPR54 and GPR103 [13], the selectivity of the compounds for these receptors was also evaluated. KGOP01, KGFF03 or KGFF09 displayed no or low affinity for GPR10, GPR54 and GPR103 (Table 5, which summarize affinity constant (K) values of KGFF compounds for GPR10, GPR54 and GPR103).

TABLE 4
Binding affinity constant (Kᵢ) values and activity agonist constant
(EC₅₀ and Eₘₐₓ) values of KGFF compounds for human DOPr, KOPr and NOPr.

| | DOPr (cAMP) | | | KOPr (cAMP) | | | NOPr (cAMP) | | |
|---|---|---|---|---|---|---|---|---|---|
| | Kᵢ | EC₅₀ | Eₘₐₓ | Kᵢ | EC₅₀ | Eₘₐₓ | Kᵢ | EC₅₀ | Eₘₐₓ |
| compound | (nM) | (nM) | (%) | (nM) | (nM) | (%) | (nM) | (nM) | (%) |
| Naloxone | 15.9 ± 2.3 | | | 8.4 ± 4.3 | | | | | |
| DPDPE | 57.1 ± 18.8 | 0.8 ± 0.3 | 100 ± 11 | nd | nd | nd | nd | nd | nd |
| Dynorphin A | nd | nd | nd | nd | 1.0 ± 0.4 | 100 ± 14 | nd | nd | nd |
| Nociceptin | nd | nd | nd | nd | nd | nd | 0.0013 ± 0.0002 | 0.568 ± 0.004 | 100 ± 3 |
| KGOP01 | 5.1 ± 0.5 | 0.16 ± 0.01 | 91 ± 2 | 37.3 ± 9.6 | >10,000 | 18 ± 1 | >10,000 | nd | nd |
| KGFF03 | 9.1 ± 0.7 | 0.34 ± 0.12 | 89 ± 6 | 108 ± 28 | 95 ± 3 | 34 ± 11 | 289 ± 3 | >10,000 | 15 ± 2 |
| KGFF09 | 186 ± 77 | 0.78 ± 0.01 | 90 ± 3 | 3.2 ± 0.8 | >10,000 | 15 ± 1 | 209 ± 2 | >10,000 | 7 ± 3 |

Kᵢ values were determined from competition binding curves using [³H]-diprenorphine for DOP and KOP receptor and [³H]-nociceptin for NOP receptor. Efficacy (Eₘₐₓ) is expressed as the percentage relative to the referent compound (DPDPE, dynorphin A and nociceptin respectively for DOP, KOP and NOP receptors). Data are mean ± SEM of at least two independent experiments performed in duplicate.
nd, not determined.

TABLE 5
Binding affinity constant (Kᵢ) values of KGFF
compounds for GPR10, GPR54 and GPR103.
Kᵢ ± SEM (nM)

| compound | GPR10 | GPR54 | GPR103 |
|---|---|---|---|
| PrRP20 | 2.1 ± 0.4 | nd | nd |
| Kp10 | nd | 0.062 ± 0.009 | nd |
| 26RFa | nd | nd | 2.04 ± 0.58 |
| KGOP01 | >10,000 | >10,000 | >10,000 |
| KGFF03 | >10,000 | 4,370 ± 960 | >10,000 |
| KGFF09 | >10,000 | 2,000 ± 300 | 1,200 ± 340 |

Data are mean ± SEM of at least two independent experiments performed in duplicate. Kᵢ values were determined from competition binding curves using [³H]-PrRP-20, [¹²⁵I]-Kp-10 and [¹²⁵I]-43RFa for GPR10, GPR54 and GPR103, respectively.
nd, not determined.

### 2.5. Acute Subcutaneous Administration of KGFF03 and KGFF09 Produces Dose-Dependent, Long-Lasting Antinociception in Mice

The acute antinociceptive activity of the new MOPr/NPFFR hybrid structures, KGFF03 and KGFF09 was then evaluated, in two mouse models of thermal acute nociception after sc. administration and compared them to the parent opioid, KGOP01. All three peptides produced time- and dose-dependent increase in tail withdrawal latencies in the tail immersion test (FIG. 3). When compared to KGOP01, KGFF03 was equipotent whereas KGFF09 was 4.5-fold less potent in inducing antinociception, in agreement with its slightly lower affinity for the MOPr (FIG. 1C).
2.6. Chronic Subcutaneous Administration of KGFF09 does not Induce Hyperalgesia Nor Analgesic Tolerance in Naïve Mice
To evaluate if the blockade of the NPFF system prevents the development of hyperalgesia and analgesic tolerance, mice were chronically administered with equianalgesic doses of either KGOP01, KGFF03 or KGFF09, as shown in the time course of analgesia on day 1 of the chronic administration scheme (FIG. 4A). When sc. administered at a dose of 1.8 μmol/kg/d, KGOP01 produced a significant and progressive decrease of the basal thermal nociceptive threshold, compared with control saline-treated animals (FIG. 48). This effect was significant from the fifth day of the daily administration and persisted until the end of the experiment. As shown in FIG. 4 (A and B), chronic administration of KGFF03 (1.2 μmol/kg/d, sc.) resulted in a similar development of thermal hypersensitivity, a trend that was absent in mice treated with KGFF09 (7.4 μmol/kg/d, sc.). In the same study, the antinociceptive effect of each compound on days 1 and 8 was measured (FIGS. 4 A and C). On day 1, KGOP01, KGFF03 and KGFF09 induced full antinociception (maximal response reaching almost the 25 s cut-off limit). On day 8, mice treated with either KGOP01 or KGFF03 showed a reduction of maximal response and a decrease in the antinociception efficacy (defined by the AUC) by more than 75% compared to day 1 (FIG. 4C), indicating that tolerance did develop upon chronic administration of these two compounds. Conversely, the maximal analgesia induced by KGFF09 was maintained from day 1 to day 8, with a decrease of analgesic efficacy (AUC) on day 8 by less than 25% as compared to day 1. These data clearly indicate that blocking NPFF1/2Rs in addition to the MOPr activation leads to potent analgesia without development of tolerance upon chronic administration.

### 2.7. KGFF09 Displays Reduced Withdrawal Syndrome and Respiratory Depression

The development of naloxone-precipitated withdrawal syndrome after chronic sc. administration to mice of KGOP01, KGFF03 and KGFF09 was further studied. Mice were treated twice a day over a 7 days period with the same doses used in previous experiments. Administration of naltrexone (1 mg/kg, sc.), 2 h after the last injection of KGOP01, induced high scores on several somatic and vegetative signs in the drug-dependent mice, as compared to the control saline-treated animals (FIG. 8, which shows effect of KGOP01, KGFF03 and KGFF09 on naltrexone-precipitated withdrawal signs after chronic exposure in mice). A similar withdrawal profile was observed for KGFF03, while all signs of withdrawal were reduced following chronic administration of KGFF09 (FIG. 8, which shows effect of KGOP01, KGFF03 and KGFF09 on naltrexone-precipitated withdrawal signs after chronic exposure in mice). Analysis of the global opiate withdrawal score revealed that withdrawal was significantly reduced (to circa 50%) after KGFF09 chronic administration, in comparison to KGFF01 and KGFF03 (FIG. 4D). This result is in agreement with a previous report, showing reduced morphine withdrawal upon administration of the NPFF1/2R antagonist RF9 [14; 48].
Respiratory depression and constipation, two of the main side effects that occur upon acute opiate administration were next studied. As shown by the measurement of respiratory frequency (FIG. 4E), KGOP01 produced significant respiratory depression compared to saline-treated animals, while KGFF03 and KGFF09 did not. This result is in agreement with previous reports suggesting that respiratory depression is strongly associated with the β-arrestin-2 recruitment by the MOPr [10; 31].

### 2.8. KGFF09 Chronic Administration Efficiently Reverses CFA-Induced Hyperalgesia

Additionally, the analgesic profile of the compounds was characterized in a mouse model of persistent inflammatory pain induced by sc. injection of CFA in the mouse tail on day 1. Animals were then daily sc. administered with equianalgesic doses of KGOP01 (1.8 μmol/kg/d), KGFF03 (1.2 μmol/kg/d) or KGFF09 (7.4 μmol/kg/d) from day 2 to day 8, and their antinociceptive activity upon thermal or mechanical nociceptive stimulation was measured on days 2, 3, 5 and 7 for the thermal stimulus, and on days 2, 4, 6, 8 for the mechanical stimulus. Measurements were performed 2 h after each daily drug injection, when analgesia was maximal (FIG. 9, which shows acute antinociceptive time-course of KGOP01, KGFF03 and KGFF09 in CFA-induced pain model). As shown in FIG. 5 (A and 8), both thermal and mechanical antinociception induced by KGOP01 and KGFF03 rapidly decreased after repeated administrations, with a decreased analgesia compared to day 1 (>75%), clearly showing that these two compounds led to the development of analgesic tolerance. In contrast, KGFF09 preserved a potent analgesic effect, with significantly less tolerance after 7 days of chronic administration (FIGS. 5 A and B). Basal nociceptive thresholds of the animals were also measured daily before injection of the compounds (FIGS. 5 C and D). The thermal hypersensitivity induced by CFA was reduced after a single administration of KGFF09, and totally reversed after the third injection (FIG. 5C), which was not the case with KGOP01 and KGFF03. Likewise, the basal mechanical nociceptive threshold of mice gradually returned to normal after repeated administration of KGFF09 (FIG. 5D), but not after KGOP01 and KGFF03. Overall, the data demonstrate that in a model of persistent inflammatory pain, blockade of NPFF1/2Rs preserves opioid analgesia upon repeated administration and allows efficient reversal of CFA-induced hyperalgesia.

3. Discussion

The driving force over the years in the opioid field has been the search for an alternative to morphine that would produce effective analgesia and would be free of undesirable side effects. New chemical approaches including the design of G protein-biased MOPr agonists and/or multifunctional ligands with mixed opioid and non-opioid activities for creating analgesics with fewer adverse effects are sought, and such drugs with improved benefit/risk profile are likely to have a significant impact [21; 24; 30].
The successful design and a thorough in vitro and in vivo characterization of G protein-biased MOPr agonists, for example KGFF03 and KGFF09, possessing additional agonist and antagonist activities at NPFF1/2Rs, respectively, were reported according to the present invention. In vivo, they display potent antinociception with reduced respiratory depression after acute systemic (sc.) administration. The major finding of this study is that following chronic administration, KGFF09 but not KGFF03 produces effective antinociception with limited OIH and analgesic tolerance, as well as a reduced withdrawal syndrome, thus demonstrating the benefits of NPFF system blockade towards MOPr agonists to limit the development of tolerance and dependence, the two major adverse effects associated with chronic administration of classical opiates.
Multitarget pharmacology, or polypharmacology, is defined as the specific binding of a compound to two or more molecular targets and relies on the observation that some biological networks are resilient to single-point perturbations, with redundant functions or compensatory mechanisms leading to the attenuation of the repeated perturbation (i.e. stimulation of the MOPr; [22; 53]. Here, the strategy aimed at developing a dual acting drug combining the analgesic efficacy of opioid agonists, while blocking the NPFF system. The latter system has previously been shown to be critically involved in neuroadaptive responses of the organism to repeated exposure to opiates, resulting in OIH and analgesic tolerance [14; 48]. To determine whether the NPFFR antagonist activity was responsible for the improved profile of KGFF09, this MOPr-NPFFR hybrid peptidomimetic was compared with its parent opioid agonist KGOP01, devoid of an NPFF pharmacophore (FIG. 1, Table 2a). Both ligands show high affinity with a full agonist activity at MOPr, paralleled by effective and long-lasting acute analgesia. However, upon chronic administration to mice, KGOP01 rapidly induced analgesic tolerance and hyperalgesia, whereas KGFF09 did not. These results appear to oppose a recent report of a bifunctional MOP-NPFF agonist, BN9, that displayed acute and chronic analgesic efficacy [27]. However, this study also shows that when co-administered with BN9, the NPFF1/2R antagonist RF9 further potentiated the analgesic effect of BN9, thus questioning the benefit of the NPFF1/2R agonist activity on opioid analgesia. In addition, KGFF03, a potent MOPr-NPFF1/2R agonist, which shows acute analgesia, was also described while it also induces tolerance and hyperalgesia in an analogous manner to the parent opioid agonist KGOP01. Altogether, the data confirm the role of the NPFF system in the compensatory mechanisms triggered by the chronic opioid stimulation and further support the concept that blockade rather than activation of this system is beneficial to opioid analgesia following chronic administration.
The beneficial effect of NPFFRs inhibition on the development of analgesic tolerance and long-lasting inflammatory hyperalgesia were further demonstrated in a model of inflammatory pain, suggesting that the endogenous NPFF system could be activated upon administration of inflammatory agents, such as CFA. This result is in agreement with a recent report showing an upregulation of NPFF and NPFF2R mRNA in the spinal cord of mice treated with CFA or carrageenan [28]. Overall, the current data therefore indicate that activation of the NPFF system might represent a common feature in the development of hyperalgesia, whether induced by inflammation or repeated opioid stimulation.
Detailed in vitro characterization of the newly designed MOP-NPFF hybrids revealed that the addition of either -Arg-Phe-NH2 (KGFF03) or -Bpa-Phe-NH2 (KGFF09) to the C-terminus of the parent opioid structure KGOP01 not only conferred the expected NPFF1/2R affinity to the designed compounds, but also shows an advantageous switch of MOPr activity towards the G protein signaling over β-arrestin-2 recruitment. Studies on β-arrestin-2 knockout mice (β-arrestin-2 KO) report less opioid-associated adverse effects, such as respiratory depression, constipation, analgesic tolerance and physical dependence, as well as higher opioid antinociceptive effects [7]. Although KGFF03 and KGFF09 are not completely devoid of the β-arrestin-2 recruitment activity, their bias appeared sufficient for alleviating respiratory depression, compared to the unbiased MOPr agonist KGOP01. This result is in agreement with previous observations made with other G protein-biased MOPr agonists [10; 31]. The lower efficacy of KGFF09 to promote MOP-induced β-arrestin-2 recruitment over G protein activation could also be responsible for less analgesic tolerance and physical dependence. However, reports on the development of analgesic tolerance induced by opioids in β-arrestin-2 KO mice or following chronic treatments with G protein-biased MOPr agonists are conflicting [1; 6; 25]. Concerning physical dependence, the severity of antagonist-precipitated withdrawal response in β-arrestin-2 KO mice was reduced only when animals were chronically treated with low doses of morphine [43] and the biased MOPr agonist TRV130 was reported to induce similar withdrawal symptoms than morphine [50]. In this study, it was shown that KGFF03, having a biased activity on the MOPr, produces similar analgesic tolerance and physical dependence to the unbiased KGOP01 parent opioid agonist, suggesting that β-arrestin-2 bias is not critical for the development of these side effects. Similarly to other biased MOPr agonists [10; 31], KGFF09 also binds to DOPr and KOPr, with DOPr agonist and KOPr antagonist activities. DOP agonists have been described to play no or limited analgesic activity in naive animals, but display potent anti-hyperalgesic activity in neuropathic and inflammatory pain models in rodents [17]. Although DOPr agonist activity is an interesting characteristic to consider when developing multitarget analgesic drugs, tolerance to DOPr-mediated analgesia has a very fast onset [42], which could limit the utility of this activity in chronic treatment. It was also found that KGFF09 displays potent KOPr antagonist activity, a property also shared by PZM21, the recently described biased MOP agonist [31]. The KOPr has been shown to display anti-MOPr activity [3; 38], and its blockade could present synergism with MOP and DOP agonist activity, leading to the observed analgesic potency of KGFF09. Moreover, as DOPr agonists and KOPr antagonists have been shown to have an antidepressant potential [29], KGFF09 may also have beneficial effects on the affective component of chronic pain syndromes.
In summary, for the first time a dual acting, G protein biased MOPr agonist—NPFFRs antagonist molecule, in particular KGFF09, was reported. The association of both properties within a single molecule gathers the beneficial effects of biased MOPr agonists on acute side effects (respiratory depression) and those of NPFFRs antagonists on chronic side effects (OIH, tolerance, withdrawal syndrome), altogether leading to a potent analgesic with an improved safety profile. Hence, the present invention supports therapeutic strategies for potent antinociceptive drugs with limited side effects upon both acute and chronic use.

### REFERENCES

[1] Altarifi A A, David B, Muchhala K H, Blough B E, Akbarali H, Negus S S. Effects of acute and repeated treatment with the biased mu opioid receptor agonist TRV130 (oliceridine) on measures of antinociception, gastrointestinal function, and abuse liability in rodents. J Psychopharmacol 2017; 31(6):730-739.
[5] Bihel F, Humbert J P, Schneider S, Bertin I, Wagner P, Schmitt M, Laboureyras E, Petit-Demouliere B, Schneider E, Mollereau C, Simonnet G, Simonin F, Bourguignon J J. Development of a peptidomimetic antagonist of neuropeptide FF receptors for the prevention of opioid-induced hyperalgesia. ACS chemical neuroscience 2015; 6(3):438-445.
[7] Bohn L M, Lefkowitz R J, Gainetdinov R R, Peppel K, Caron M G, Lin F T. Enhanced morphine analgesia in mice lacking beta-arrestin 2. Science (New York, N.Y. 1999; 286(5449):2495-2498.
[8] Celerier E, Rivat C, Jun Y, Laulin J P, Larcher A, Reynier P, Simonnet G. Long-lasting hyperalgesia induced by fentanyl in rats: preventive effect of ketamine. Anesthesiology 2000; 92(2):465-472.
[10] DeWire S M, Yamashita D S, Rominger D H, Liu G, Cowan C L, Graczyk T M, Chen X T, Pitis P M, Gotchev D, Yuan C, Koblish M, Lark M W, Violin J D. A G protein-biased ligand at the mu-opioid receptor is potently analgesic with reduced gastrointestinal and respiratory dysfunction compared with morphine. The Journal of pharmacology and experimental therapeutics 2013; 344(3):708-717.
[12] Elhabazi K, Ayachi S, Hien B, Simonin F. Assessment of morphine-induced hyperalgesia and analgesic tolerance in mice using thermal and mechanical nociceptive modalities. Journal of visualized experiments: JoVE 2014 (89):e51264.
[13] Elhabazi K, Humbert J P, Bertin I, Schmitt M, Bihel F, Bourguignon J J, Bucher B, Becker J A, Sorg T, Meziane H, Petit-Demouliere B, Hien B, Simonin F. Endogenous mammalian RF-amide peptides, including PrRP, kisspeptin and 26RFa, modulate nociception and morphine analgesia via NPFF receptors. Neuropharmacology 2013; 75C:164-171.
[14] Elhabazi K, Trigo J M, Mollereau C, Mouledous L, Zajac J M, Bihel F, Schmitt M, Bourguignon J J, Meziane H, Petit-demouliere B, Bockel F, Maldonado R, Simonin F. Involvement of neuropeptide FF receptors in neuroadaptive responses to acute and chronic opiate treatments. British journal of pharmacology 2012; 165(2):424-435.
[16] Findeisen M, Rathmann D, Beck-Sickinger A G. Structure-activity studies of RFamide peptides reveal subtype-selective activation of neuropeptide FF1 and FF2 receptors. ChemMedChem 2011; 6(6):1081-1093.
[17] Gaveriaux-Ruff C, Kieffer B L. Delta opioid receptor analgesia: recent contributions from pharmacology and molecular approaches. Behavioural pharmacology 2011; 22(5-6):405-414.
[18] Gealageas R, Schneider S, Humbert J-P, Bertin I, Schmitt M, Laboureyras E, Dugave C, Mollereau C, Simonnet G, Bourguignon J-J, Simonin F, Bihel F. Development of sub-nanomolar dipeptidic ligands of neuropeptide FF receptors. Bioorganic & medicinal chemistry letters 2012; 22(24):7471-7474.
[20] Guillemyn K, Kleczkowska P, Lesniak A, Dyniewicz J, Van der Poorten O, Van den Eynde I, Keresztes A, Varga E, Lai J, Porreca F, Chung N N, Lemieux C, Mika J, Rojewska E, Makuch W, Van Duppen J, Przewlocka B, Vanden Broeck J, Lipkowski A W, Schiller P W, Tourwe D, Ballet S. Synthesis and biological evaluation of compact, conformationally constrained bifunctional opioid agonist—neurokinin-1 antagonist peptidomimetics. European journal of medicinal chemistry 2015; 92:64-77.
[21] Gunther T, Dasgupta P, Mann A, Miess E, Kliewer A, Fritzwanker S, Steinborn R, Schulz S. Targeting multiple opioid receptors—improved analgesics with reduced side effects? British journal of pharmacology 2017; in press.
[22] Hopkins A L. Network pharmacology: the next paradigm in drug discovery. Nature chemical biology 2008; 4(11):682-690.
[23] Journigan V B, Mesangeau C, Vyas N, Eans S O, Cutler S J, McLaughlin J P, Mollereau C, McCurdy C R. Nonpeptide small molecule agonist and antagonist original leads for neuropeptide FF1 and FF2 receptors. Journal of medicinal chemistry 2014; 57(21):8903-8927.
[24] Kleczkowska P, Lipkowski A W, Tourwe D, Ballet S. Hybrid opioid/non-opioid ligands in pain research. Current pharmaceutical design 2013; 19(42):7435-7450.
[25] Koblish M, Carr R, 3rd, Siuda E R, Rominger D H, Gowen-MacDonald W, Cowan C L, Crombie A L, Violin J D, Lark M W. TRV0109101, a G Protein-Biased Agonist of the micro-Opioid Receptor, Does Not Promote Opioid-Induced Mechanical Allodynia following Chronic Administration. The Journal of pharmacology and experimental therapeutics 2017; 362(2):254-262.
[27] Li N, Han Z L, Wang Z L, Xing Y H, Sun Y L, Li X H, Song J J, Zhang T, Zhang R, Zhang M N, Xu B, Fang Q, Wang R. BN-9, a chimeric peptide with mixed opioid and neuropeptide FF receptor agonistic properties, produces nontolerance-forming antinociception in mice. British journal of pharmacology 2016; 173(11):1864-1880.
[28] Lin Y T, Liu H L, Day Y J, Chang C C, Hsu P H, Chen J C. Activation of NPFFR2 leads to hyperalgesia through the spinal inflammatory mediator CGRP in mice. Experimental neurology 2017; 291:62-73.
[29] Lutz P E, Kieffer B L. Opioid receptors: distinct roles in mood disorders. Trends in neurosciences 2013; 36(3):195-206.
[30] Madariaga-Mazon A, Marmolejo-Valencia A F, Li Y, Toll L, Houghten R A, Martinez-Mayorga K. Mu-Opioid receptor biased ligands: A safer and painless discovery of analgesics? Drug discovery today 2017; 22(11):1719-1729.
[31] Manglik A, Lin H, Aryal D K, McCorvy J D, Dengler D, Corder G, Levit A, Kling R C, Bernat V, Hubner H, Huang X P, Sassano M F, Giguere P M, Lober S, Da D, Scherrer G, Kobilka B K, Gmeiner P, Roth B L, Shoichet B K. Structure-based discovery of opioid analgesics with reduced side effects. Nature 2016; 537(7619):185-190.
[33] Mazarguil H, Gouarderes C, Tafani J A, Marcus D, Kotani M, Mollereau C, Roumy M, Zajac J M. Structure-activity relationships of neuropeptide FF: role of C-terminal regions. Peptides 2001; 22(9):1471-1478.
[34] Michel G, Matthes H W, Hachet-Haas M, El Baghdadi K, de Mey J, Pepperkok R, Simpson J C, Galzi J L, Lecat S. Plasma membrane translocation of REDD1 governed by GPCRs contributes to mTORC1 activation. Journal of cell science 2014; 127(Pt 4):773-787.
[35] Nguyen T, Decker A M, Langston T L, Mathews K M, Siemian J N, Li J X, Harris D L, Runyon S P, Zhang Y. Discovery of Novel Proline-Based Neuropeptide F F Receptor Antagonists. ACS chemical neuroscience 2017; 8(10):2290-2308.
[38] Pan Z Z. mu-Opposing actions of the kappa-opioid receptor. Trends in pharmacological sciences 1998; 19:94-98.
[39] Papaleo F, Contarino A. Gender- and morphine dose-linked expression of spontaneous somatic opiate withdrawal in mice. Behavioural brain research 2006; 170(1):110-118.
[41] Pradhan A A, Becker J A, Scherrer G, Tryoen-Toth P, Filliol D, Matifas A, Massotte D, Gaveriaux-Ruff C, Kieffer B L. In vivo delta opioid receptor internalization controls behavioral effects of agonists. PloS one 2009; 4(5):e5425.
[42] Pradhan A A, Walwyn W, Nozaki C, Filliol D, Erbs E, Matifas A, Evans C, Kieffer B L. Ligand-directed trafficking of the delta-opioid receptor in vivo: two paths toward analgesic tolerance. J Neurosci 2010; 30(49):16459-16468.
[43] Raehal K M, Bohn L M. beta-arrestins: regulatory role and therapeutic potential in opioid and cannabinoid receptor-mediated analgesia. Handbook of experimental pharmacology 2014; 219:427-443.
[45] Roeckel L A, Utard V, Reiss D, Mouheiche J, Maurin H, Robe A, Audouard E, Wood J N, Goumon Y, Simonin F, Gaveriaux-Ruff C. Morphine-induced hyperalgesia involves mu opioid receptors and the metabolite morphine-3-glucuronide. Scientific reports 2017; 7(1):10406.
[46] Schneider S, Ftouni H, Niu S, Schmitt M, Simonin F, Bihel F. Rapid and scalable synthesis of innovative unnatural alpha,beta or gamma-amino acids functionalized with tertiary amines on their side-chains. Organic & biomolecular chemistry 2015; 13(25):7020-7026.
[48] Simonin F, Schmitt M, Laulin J-P, Laboureyras E, Jhamandas J H, MacTavish D, Matifas A, Mollereau C, Laurent P, Parmentier M, Kieffer B L, Bourguignon J-J, Simonnet G. RF9, a potent and selective neuropeptide FF receptor antagonist, prevents opioid-induced tolerance associated with hyperalgesia. Proc Natl Acad Sci USA 2006; 103(2):466-471.
[49] Simonin F, Valverde O, Smadja S, Slowe S, Kitchen I, Dierich A, Le Meur M, Rogues B P, Maldonado R, Kieffer B L. Disruption of the k-opioid receptor gene in mice enhances sensitivity to chemical visceral pain, impairs pharmacological actions of the selective k-agonist U-50,488H and attenuates morphine withdrawal. EMBO J 1998; 17:886-897.
[50] Siuda E R, Carr R, 3rd, Rominger D H, Violin J D. Biased mu-opioid receptor ligands: a promising new generation of pain therapeutics. Curr Opin Pharmacol 2017; 32:77-84.
[53] Talevi A. Multi-target pharmacology: possibilities and limitations of the “skeleton key approach” from a medicinal chemist perspective. Frontiers in pharmacology 2015; 6:205.

1. A molecule comprising the following structure:
X1-X2-X3-X4-X5-X6-T
wherein
X1 has the following structure: 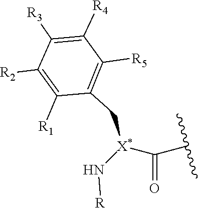
wherein
R2, R₃ and R₄ are, independently at each occurrence H, OH, NH₂, CH₃, CONH₂, or COCH₃
R₁ and R₅ are, independently at each occurrence, H or Me
R is H, alkyl or C(═N)NH₂
X is N or CRa, wherein Ra is H or Me;
X2 is a natural or non-natural amino acid residue, or derivative thereof, including homologated amino acids, aza amino acids,
or X1-X2 represent together the following structure: 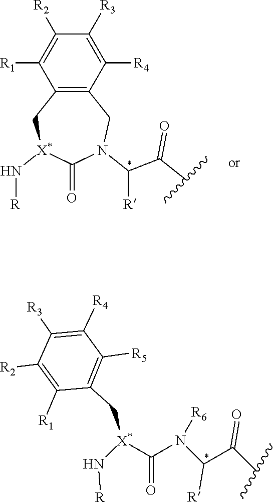
R2, R₃ and R4 are, independently at each occurrence H, OH, NH2, CH₃, CONH2, or COCH₃
R₁, R₅ and R₆ are, independently at each occurrence, H or Me
R is H or C(═N)NH₂
R′ is H or a group of atoms, including natural and non-natural amino acid side chains
X is N or CRa, wherein Ra is H or Me,
X3 is a natural or non-natural amino acid residue,
X4 is one to five natural or non-natural amino acid residue or a derivative thereof,
X5 has the following structure: 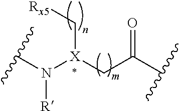
wherein Rx₅ is a cyclic or acyclic tertiary amine group linked by the nitrogen atom of the tertiary amine group,
wherein: if m=0, then n=2, 3, or 4;
if m=1, or 2, then n=1
wherein X is CRa or N, wherein Ra is H or Me,
wherein R′ is H or Alkyl (for example Me, Et);
wherein each carbon atom of (CH2)n and (CH2)m can be substituted independently of each other;
X6 is a natural or non-natural amino acid residue
T is a chemical terminal group of atoms, 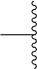
represents a covalent link.

2. The molecule of claim 1 wherein X1 is 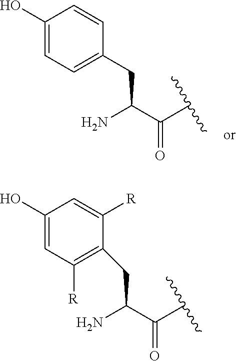
wherein R is alkyl (methyl, ethyl),
or 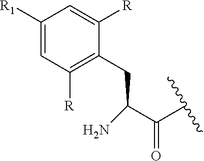
wherein R1 is NH2, CH₃, CONH₂, COCH₃ and R is H or alkyl (methyl, ethyl), 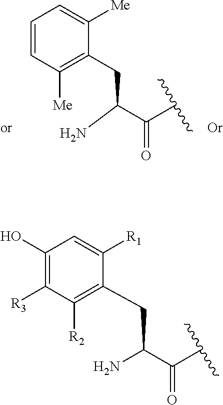
wherein:
R₁ is Me, R2 is Et and R₃ is H or
R₁ is Me, R2 is iPr and R₃ is H or
R₁, R2 and R₃ are Me.

3. The molecule of claim 1 wherein X2 is 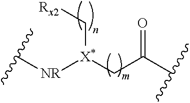
wherein X is CRa or N and Ra is H or Me;
wherein R is H or alkyl (typically Me);
wherein Rₓ₂ is H, alkyl chain bearing an substituted amino group or a substituted guanidine group,
wherein: if m=0, then n=2, 3, or 4;
if m=1, or 2, then n=1.

4. The molecule of claim 1 wherein X2 has the following structure: 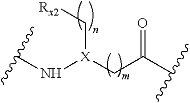
wherein Rₓ₂ is a cyclic or acyclic tertiary amine group linked by the nitrogen atom of the tertiary amine group or is a phenyl group optionally substituted by an alkyl group or an amino acid side chain,
wherein: if m=0, then n=2, 3, or 4;
if m=1, or 2, then n=1
wherein each carbon atom of (CH2)n and (CH2)m can be substituted independently of each other.

5. The peptide sequence of claim 1 wherein X3 has the following structure: 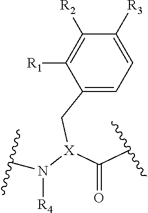
wherein R4 is an amino acid side chain; R1, R2 and R₃ are each independently H, halogen, alkyl, alkenyl, (hetero)aryl;
wherein X is CRa or N, wherein Ra is H or Me.

6. The molecule of claim 1 wherein X3-X4 together represent the following structure: 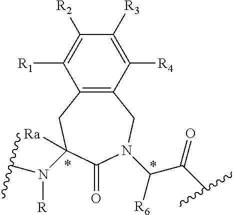
Wherein R is an amino acid side chain
Ra is H or Me
R₁ to R₄ and R₆ are each independently H, halogen, alkyl, alkenyl, (hetero)aryl; 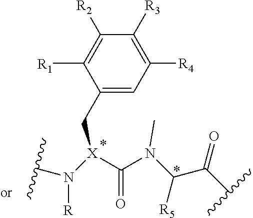
wherein X is CRa or N, wherein Ra is H or Me
wherein R₄ is an amino acid side chain
Ra is H or Me
R₁ to R₅ are each independently H, halogen, alkyl, alkenyl, (hetero)aryl.

7. The molecule of claim 1 wherein X4 has the following structure:
α-amino acids 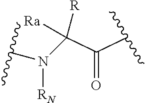
wherein R is an amino acid side chain or derivative thereof
wherein Ra is H or Me
wherein R_{N} is H or alkyl
β3-homo-amino acids 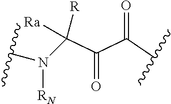
wherein R is an amino acid side chain or derivative thereof
wherein Ra is H or Me
wherein R_{N} is H or alkyl
β2-homo-amino acids 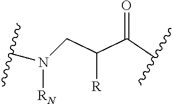
wherein R is an amino acid side chain or derivative thereof
wherein R_{N} is H or alkyl
aza-amino acids 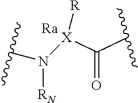
wherein R is an amino acid side chain or derivative thereof
wherein Ra is H or Me
wherein R_{N} is H or alkyl.

8. The molecule of claim 1, wherein X4 comprises one of the following group C-terminal group forming a natural or non-natural amino acid residue or a derivative thereof: 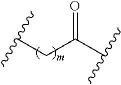
wherein 0≤m≤5; 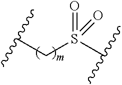
wherein 0≤m≤5; 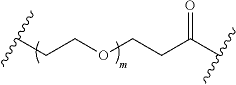
wherein 1≤m≤3; 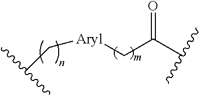
wherein 0≤m≤3 and 0≤n≤3 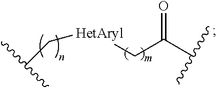
wherein 0≤m≤3 and 0≤n≤3. 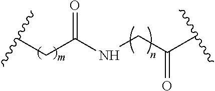
wherein 0≤m≤5 and 0≤n≤5 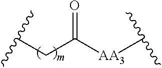
wherein 0≤m≤5 and AA₃ represents one or two residues selected each independently among the list of residues X5 and X6,
wherein each carbon atom of (CH₂)n and (CH₂)m can be substituted independently of each other.

9. The molecule of claim 1 wherein Rₓ₅ is selected from the group consisting of:
a cyclic or acyclic guanidine bearing substituents: 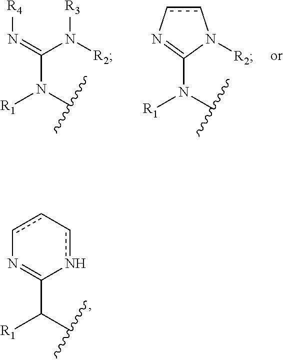
a cyclic or acyclic urea or thiourea bearing substituents: 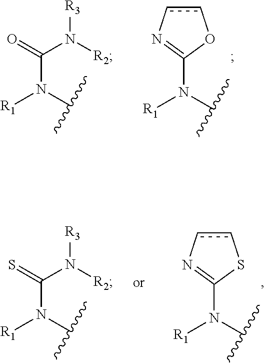
and
a cyclic or acyclic tertiary amine group: 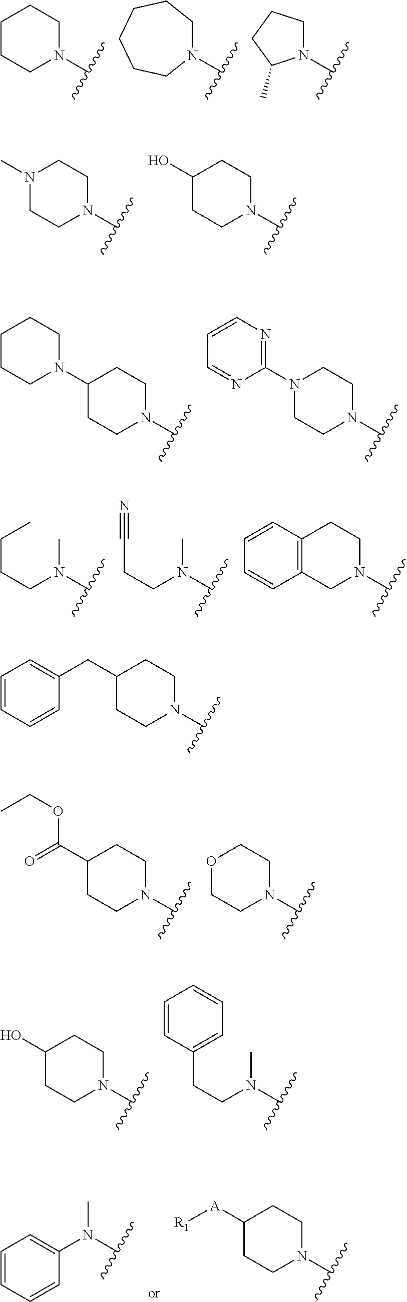
wherein A is (CH₂)ₙ with n=0, 1, 2, 3, O, S, or NH,
wherein each carbon atom of (CH₂)n is optionally substituted independently of each other,
wherein R1 is Aryl or Heteroaryl, optionally substituted.

10. The molecule of claim 1, wherein X6 is selected from the group consisting of:
a natural or non-natural amino acids of configuration L or D including one of the following structure:
Gly, Ala, Val, Ile, Leu, Nle, cHex, Phe, Hphe, Tyr, Trp, Asn, Gln, Pro
Arg, Lys, Cys, Met, Asp, Glu; and
a bridged amino acids including:

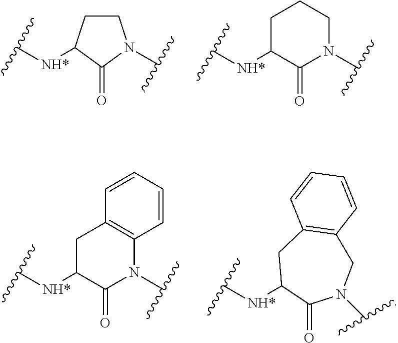

11. The molecule of claim 1 wherein said terminal group T is selected from the group consisting of:
H, Alkyl, (CH₂)ₙ-Aryl (when X6 is a bridged amino acid), and
NH₂, NH—R or cyclic/acyclic NR₁R₂ wherein R, R₁, R₂ is H, Alkyl or (CH₂)ₙ-Aryl.

12. The molecule of claim 1 wherein said molecule comprises from 6 to 10 amino acid residues or derivative thereof.

13. The molecule of claim 1 wherein X1-X2-X3-X4 represent a opioid peptide-based peptide analogue structure or a dermophin peptide based peptide analogue structure.

14. A molecule according to claim 1, wherein said molecule is binding MOR and NPFFR.

15. A molecule according to claim 1, wherein said molecule is an NPFFR1 or NPFFR2 antagonist, and in particular a NPFFR1 and NPFFR2 antagonist.

16. A method of treating an animal or human body, said method comprising administering to said animal or human body of an effective amount of at least one molecule as defined in claim 1.

17. The method according to claim 16, wherein said method is a method of treatment of pain and/or hyperalgesia.

18. The method according to claim 16, wherein said method is a method of treatment of a disease or condition associated with MOR.

19. The method according to claim 16, wherein said method is a method of treatment of a disease or condition associated with NPFFR1 and/or NPFFR2.

20. The method according to claim 16, wherein said method is method of treatment of behavioral and somatic signs of opioid withdrawal syndrome.

21. A pharmaceutical composition comprising at least one molecule according to claim 1 and one or more pharmaceutically acceptable excipients.

22. A pharmaceutical composition comprising at least one molecule according to claim 2 and one or more pharmaceutically acceptable excipients.

23. A pharmaceutical composition comprising at least one molecule according to claim 6 and one or more pharmaceutically acceptable excipients.

24. A pharmaceutical composition comprising at least one molecule according to claim 8 and one or more pharmaceutically acceptable excipients.

25. A pharmaceutical composition comprising at least one molecule according to claim 9 and one or more pharmaceutically acceptable excipients.
